(19) United States
(12) Patent Application Publication
(10) Pub. No.: US 2023/0398205 A1
(43) Pub. Date: Dec. 14, 2023

(54) SARS-COV-2 IMMUNODOMINANT PEPTIDE CONSTRUCTS AND USES THEREOF

(71) Applicants: TScan Therapeutics, Inc., Waltham, MA (US); AHS Hospital Corp., Morristown, NJ (US)

(72) Inventors: Tomasz KULA, Brookline, MA (US); Gavin MacBeath, Wakefield, MA (US); Andrew P. Ferretti, Waltham, MA (US)

(21) Appl. No.: 18/035,369

(22) PCT Filed: Nov. 12, 2021
(86) PCT No.: PCT/US2021/059050

§ 371 Date: May 4, 2023

Related U.S. Application Data
(60) Provisional application No. 63/113,024, filed on Nov. 12, 2020.

(51) Int. Cl. A61K 39/215; C07K 14/005; G01N 33/569; G01N 33/50

(52) U.S. Cl. CPC A61K 39/215; C07K 14/005; G01N 33/56972; G01N 33/5091; A61K 2039/70

(57) ABSTRACT

Provided herein are methods and compositions for the treatment and/or prevention of COVID-19 through the induction of an immune response against identified SARS-COV-2 immunodominant peptides.

### CROSS-REFERENCE TO RELATED APPLICATIONS

This application claims the benefit of U.S. Provisional Application No. 63/113,024, filed on 12 Nov. 2020; the entire contents of said application are incorporated herein in their entirety by this reference.

### BACKGROUND OF THE INVENTION

Coronavirus Disease 2019, or COVID-19, is a global pandemic caused by infections with Severe Acute Respiratory Syndrome (SARS)-CoV-2 (SARS-CoV-2) virus that has claimed >500,000 lives world-wide and has affected millions more. SARS-CoV-2 is the seventh coronavirus known to infect humans; SARS-CoV, MERS-CoV and SARS-CoV-2 can cause severe disease, whereas HKU1, NL63, OC43 and 229E are associated with mild symptoms. Developing effective vaccines and therapies requires understanding how the adaptive immune response recognizes and clears the virus and how the interplay between the virus and the immune system affects the pathology of the disease. To date, most efforts have focused on the B cell-mediated antibody response to the virus, but less is understood about how cytotoxic CD8+ T cells recognize and clear infected cells.

CD8 T cells play a critical role in providing protection from various pathogens, and a mounting body of evidence demonstrates that this is the case for SARS-CoV-2. Studies of the closely-related SARS-CoV reveal that CD8 T cells provide protection during acute infection in animal models and that memory CD8 T cells persist longer than humoral responses following acute infection in humans. During SARS-CoV-2 infection, the magnitude of the CD8 T cell response correlates with a milder disease course, suggesting a protective role. Notably, SARS-CoV-2-reactive T cells have been observed in the absence of antiviral antibodies in patients exposed to SARS-CoV-2 who cleared the virus asymptomatically, further arguing for a protective role of T cells.

Current SARS-CoV-2 vaccine development efforts are focused primarily at generating neutralizing antibodies against the S protein. The vast majority of vaccines in clinical development (and all of the Ph II/III candidates in the US) use only the S protein as an antigen. As a result, these vaccine candidates are missing most of the antigens recognized by CD8 T cells during natural infection. Indeed, careful tracking of T cell responses in patients vaccinated with SARS-CoV-2 S protein vaccines have revealed variable and weak CD8 T cell responses. Next-generation vaccines incorporating a broader range of antigens are needed to better engage CD8 T cell responses in the hope of providing more robust and durable protection from SARS-CoV-2. To date, SARS-CoV-2 vaccines in development have resulted in weak and variable CD8 responses after vaccination (Mateus et al. (2021) Science 374:eabj9853).

### SUMMARY OF THE INVENTION

The present invention is based, at least in part, on the discovery of SARS-CoV-2 immunodominant peptides. Importantly, some of these immunogenic peptides can elicit T cell response across patients, for example when they are concatenated, when they are arranged otherwise in a polypeptide, or when they are combined with other peptides on a construct (e.g., nucleic acid vector constructs, such as those that maximize vector size to T cell response efficacy by packing in optimal immunodominant epitopes for intracellular antigen expression).

In some aspects, immunogenic polypeptides comprise at least two peptide epitopes selected from Table 1A, 1B, 1C, 1D, 1E, and/or 1F. In certain aspects, said at least two peptide epitopes are in a concatenated order in the immunogenic polypeptides, optionally wherein at least one or more immunodominant epitopes are present in more than one copy. Numerous embodiments are further provided that can be applied to any aspect of the present invention and/or combined with any other embodiment described herein. For example, in some embodiments, at least one or more immunodominant epitopes are present in more than one copy, such as being present in 2 copies, 3 copies, 4 copies, 5 copies, 6 copies, or more, or any range in between inclusive, such as 2 copies of one immunodominant epitope, 3 copies of another immunodominant epitope, a single copy of still a third immunodominant epitope, etc. In some embodiments, the immunogenic polypeptides comprise at least 3, 4, 5, 6, 7, 8, 9, 10, 11, 12, 13, 14, 15, 16, 17, 18, 19, 20, 21, 22, 23, 24, 25, 26, 27, 28, 29, or more of said peptide epitopes, optionally wherein the immunogenic polypeptide comprises at least one, two, and/or three immunodominant epitopes per each of HLA-A*02, HLA-A*03, HLA-A*01, HLA-A*11, HLA-A*24, and HLA-B*07 (for example, one immunodominant epitopes per each listed HLA, two immunodominant epitopes per each listed HLA, three immunodominant epitopes per each listed HLA, or any range in between, inclusive, such as one immunodominant epitope for each of HLA-A*02 and HLA-A*03 plus two immunodominant epitopes for each of HLA-A*01 and HLA-A*11, and three immunodominant epitopes for each of HLA-A*24 and HLA-B*07, etc.). In some embodiments, the immunogenic polypeptides comprise 3 peptide epitopes from each of Table 1A, 1B, 1C, 1D, 1E, and 1F. In some embodiments, the immunogenic polypeptides further comprise a linker between the peptide epitopes. In certain embodiments, the linker comprises at least three amino acids for each of the peptide epitopes, wherein said at least three amino acids are those that are contiguous with their respective peptide epitopes. In certain embodiments, the linker is a proteasomal cleavage motif. In some embodiments, the immunogenic polypeptides further comprise one or more full-length SARS-CoV-2 proteins selected from the group consisting of Orf1ab, M, N, Orf3a, and S, or one or more protein fragments thereof, optionally wherein the fragments of the one or more full-length SARS-CoV-2 protein(s) encompass the SARS-CoV-2 protein(s) without encoding the functional SARS-CoV-2 protein(s). In some embodiments, the immunogenic polypeptides further comprise ribosomal stop/restart segment, IRES segment, and/or a post-translational cleavage segment, optionally wherein the post-translational cleavage segment is a P2A segment. In some embodiments, the immunogenic polypeptides comprise any one of the amino acid sequences provided in a Table described herein, such as Table 1G or Table 11.

In certain aspects, the immunogenic polypeptides comprise at least two peptide fragments each of which comprises at least two of said peptide epitopes, wherein said at least two of said peptide epitopes in each peptide fragment are derived from the same protein of SARS-CoV-2.

As described above, numerous embodiments are further provided that can be applied to any aspect of the present invention and/or combined with any other embodiment described herein. For example, in some embodiments, said at least two peptide fragments are derived from the N protein, the M protein, the ORF1a/b protein, or the ORF3a protein of SARS-CoV-2. In some embodiments, the immunogenic polypeptides comprise at most 6 of said peptide fragments. In some embodiments, the immunogenic polypeptides further comprise one or more full-length SARS-CoV-2 proteins selected from the group consisting of Orf1ab, M, N, Orf3a, and S, or one or more protein fragments thereof, optionally wherein the fragments of the one or more full-length SARS-CoV-2 protein(s) encompass the SARS-CoV-2 protein(s) without encoding the functional SARS-CoV-2 protein(s). In some embodiments, the immunogenic polypeptides further comprise a ribosomal stop/restart segment, IRES segment, and/or a post-translational cleavage segment, optionally wherein the post-translational cleavage segment is a P2A segment. In some embodiments, the immunogenic polypeptides comprise any one of the amino acid sequences provided in a Table described herein, such as Table 1H or Table 1J. In some embodiments, the immunogenic polypeptides are capable of eliciting a T cell response in vitro or in vivo, optionally wherein the T cell response is determined by a tetramer staining assay, T cell activation assay, CD137 staining assay, intracellular IFNgamma (IFNg) staining assay, cytokine release assay, and/or T cell proliferation assay.

Numerous embodiments are further provided that may be applied to any aspect of the present invention and/or combined with any other embodiment described herein. For example, in one embodiment, the immunogenic peptide is derived from a SARS-CoV-2 protein, optionally wherein the immunogenic peptide is 8, 9, 10, 11, 12, 13, 14, or 15 amino acids in length. In another embodiment, the SARS-CoV-2 protein is selected from the group consisting of orf1a/b, S protein, N protein, M protein, orf3a, and orf7a. In still another embodiment, the immunogenic peptide is capable of eliciting a T cell response in a subject. In some embodiments, the immunogenic peptides comprise a peptide epitope selected from Table 1A, 1B, 1C, 1D, 1E, and/or 1F.

In still another aspect, an immunogenic composition comprising at least one immunogenic peptide described herein (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, or more, or any range in between, inclusive, such as 1-5 peptides), optionally wherein the immunogenic composition further comprises 1) one or more full-length SARS-CoV-2 proteins selected from the group consisting of Orf1ab, M, N, Orf3a, and S, or one or more protein fragments thereof, optionally wherein the fragments of the one or more full-length SARS-CoV-2 protein(s) encompass the SARS-CoV-2 protein(s) without encoding the functional SARS-CoV-2 protein(s) and/or 2) an adjuvant, is provided.

Numerous embodiments are further provided that may be applied to any aspect of the present invention and/or combined with any other embodiment described herein. For example, in one embodiment, the immunogenic composition is capable of eliciting a T cell response in vitro and/or in vivo. In another embodiment, the T cell response is determined by a tetramer staining assay, T cell activation assay, CD137 staining assay, intracellular IFNg staining assay, cytokine release assay, and/or T cell proliferation assay.

In yet another aspect, a composition comprising an immunogenic polypeptide that comprises at least two peptide epitopes selected from Table 1A, 1B, 1C, 1D, 1E, and/or 1F, and an MHC molecule, is provided.

Numerous embodiments are further provided that may be applied to any aspect of the present invention and/or combined with any other embodiment described herein. For example, in one embodiment, the MHC molecule is a MHC multimer, optionally wherein the MHC multimer is a tetramer. In another embodiment, the MHC molecule is an MHC class I molecule. In still another embodiment, the MHC molecule comprises an MHC alpha chain that is an HLA serotype selected from the group consisting of HLA-A*02, HLA-A*03, HLA-A*01, HLA-A*11, HLA-A*24, and HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. Sequences, characteristics, structural information, functional information, binding partners, and the like for these and other HLA alleles are well-known in the art (see, e.g., the World Wide Web at hla.alleles.org/nomenclature/index.html, hla.alleles.org/data/hla-a.html, and hla.alleles.org/data/hla-b.html).

In another aspect, a stable MHC-peptide complex, comprising a peptide epitope selected from Table 1A, 1B, 1C, 1D, 1E, and/or 1F in the context of an MHC molecule, is provided.

Numerous embodiments are further provided that may be applied to any aspect of the present invention and/or combined with any other embodiment described herein. For example, in one embodiment, the MHC molecule is a MHC multimer, optionally wherein the MHC multimer is a tetramer. In another embodiment, the MHC molecule is a MHC class I molecule. In still another embodiment, the MHC molecule comprises an MHC alpha chain that is an HLA serotype selected from the group consisting of HLA-A*02, HLA-A*03, HLA-A*01, HLA-A*11, HLA-A*24, and HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. In yet another embodiment, the peptide epitope and the MHC molecule are covalently linked and/or wherein the alpha and beta chains of the MHC molecule are covalently linked. In another embodiment, the stable MHC-peptide complex comprises a detectable label, optionally wherein the detectable label is a fluorophore.

In still another aspect, an immunogenic composition comprising a stable MHC-peptide complex described herein, and an adjuvant, is provided.

In yet another aspect, an isolated nucleic acid that encodes an immunogenic polypeptide described herein, or a complement thereof, optionally wherein the isolated nucleic acid is DNA, RNA, mRNA, cDNA, self-replicating, cyclized, concatamerized, comprises a 5′ untranslated region (5′UTR) and/or 3′UTR derived from a gene of interest, such as hemoglobin A, comprises an expression promoter, comprises an internal ribosome entry site (IRES), and/or comprises a self-cleaving 2A peptide, such as P2A or T2A, is provided.

In another aspect, a vector comprising an isolated nucleic acid described herein, is provided. In some embodiments, the vector is an expression vector.

It is contemplated that a basic nucleic acid encoding an immunogenic polypeptide described herein, or a vector comprising same, may be used in any aspect or embodiment of the present invention where the immunogenic polypeptide is desired (e.g., the nucleic acid encodes and produces the immunogenic polypeptide). The nucleic acid is an immunogenic composition because of the encoded and produced immunogenic polypeptide and not due to the nucleic acid per se.

In still another aspect, a cell that a) comprises an isolated nucleic acid described herein, b) comprises a vector described herein, and/or c) produces one or more immunogenic polypeptides described herein and/or presents at the cell surface one or more stable MHC-peptide complexes described herein, optionally wherein the cell is genetically engineered, is provided.

In still another aspect, a binding moiety that specifically binds an immunogenic peptide described herein and/or a stable MHC-peptide complex described herein, optionally wherein the binding moiety is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain (optionally further comprising a transmembrane domain and an effector domain that is intracellular), is provided.

In yet another aspect, a device or kit comprising a) one or more immunogenic polypeptides described herein and/or b) one or more stable MHC-peptide complexes described herein, said device or kit optionally comprising a reagent to detect binding of a) and/or b) to a T cell receptor, is provided.

In another aspect, a method of detecting T cells that bind a stable MHC-peptide complex comprising: (a) contacting a sample comprising T cells with a stable MHC-peptide complex described herein; and (b) detecting binding of T cells to the stable MHC-peptide complex, optionally further determining the percentage of stable MHC-peptide-specific T cells that bind to the stable MHC-peptide complex, is provided.

Numerous embodiments are further provided that may be applied to any aspect of the present invention and/or combined with any other embodiment described herein. For example, in one embodiment, the sample comprises peripheral blood mononuclear cells (PBMCs). In another embodiment, the T cells are CD8+ T cells. In still another embodiment, the detecting and/or determining is performed using fluorescence activated cell sorting (FACS), enzyme linked immunosorbent assay (ELISA), radioimmune assay (RIA), immunochemically, Western blot, or intracellular flow assay. In still another embodiment, the sample comprises T cells contacted with, or suspected of having been contacted with, one or more SARS-CoV-2 proteins or fragments thereof.

In still another aspect, a method of determining whether a subject has exposure to and/or protection from SARS-CoV-2 comprising a) incubating a cell population comprising T cells obtained from the subject with an immunogenic polypeptide described herein or a stable MHC-peptide complex described herein; and b) detecting the presence or level of reactivity, wherein the presence of or a higher level of reactivity compared to a control level indicates that the subject has exposure to and/or protection from SARS-CoV-2, is provided.

In yet another aspect, a method for predicting the clinical outcome of a subject afflicted with SARS-CoV-2 infection comprising a) determining the presence or level of reactivity between T cells obtained from the subject and one more immunogenic peptides described herein or one or more stable MHC-peptide complexes described herein; and b) comparing the presence or level of reactivity to that from aa control, wherein the control is obtained from a subject having a good clinical outcome, wherein the presence or a higher level of reactivity in the subject sample as compared to the control indicates that the subject has a good clinical outcome, is provided.

In another aspect, a method of assessing the efficacy of a SARS-CoV-2 therapy comprising a) determining the presence or level of reactivity between T cells obtained from the subject and one more immunogenic peptides described herein or one or more stable MHC-peptide complexes described herein, in a first sample obtained from the subject prior to providing at least a portion of the SARS-CoV-2 therapy to the subject, and b) determining the presence or level of reactivity between the one more immunogenic peptides described herein, or the one or more stable MHC-peptide complexes described herein, and T cells obtained from the subject present in a second sample obtained from the subject following provision of the portion of the SARS-CoV-2 therapy, wherein the presence or a higher level of reactivity in the second sample, relative to the first sample, is an indication that the therapy is efficacious for treating SARS-CoV-2 in the subject, is provided.

Numerous embodiments are further provided that may be applied to any aspect of the present invention and/or combined with any other embodiment described herein. For example, in one embodiment, the level of reactivity is indicated by a) the presence of binding and/or b) T cell activation and/or effector function, optionally wherein the T cell activation or effector function is T cell proliferation, killing, or cytokine release. In another embodiment, the method further comprises repeating steps a) and b) at a subsequent point in time, optionally wherein the subject has undergone treatment to ameliorate SARS-CoV-2 infection between the first point in time and the subsequent point in time. In still another embodiment, the T cell binding, activation, and/or effector function is detected using fluorescence activated cell sorting (FACS), enzyme linked immunosorbent assay (ELISA), radioimmune assay (RIA), immunochemically, Western blot, or intracellular flow assay. In yet another embodiment, the control level is a reference number. In another embodiment, the control level is a level of a subject without exposure to SARS-CoV-2.

In still another aspect, a method of preventing and/or treating SARS-CoV-2 infection in a subject comprising administering to the subject a therapeutically effective amount of an immunogenic composition comprising and/or encoding one or more immunogenic polypeptides, and/or a cell described herein, is provided.

Numerous embodiments are further provided that may be applied to any aspect of the present invention and/or combined with any other embodiment described herein. For example, in one embodiment, the immunogenic composition comprises a nucleic acid that encodes an immunogenic polypeptide described herein, such as an immunogenic polypeptide that comprises at least two peptide epitopes selected from Table 1A, 1B, 1C, 1D, 1E, and/or 1F. In an embodiment, the immunogenic peptide is derived from a SARS-CoV-2 protein, optionally wherein the immunogenic peptide is 8, 9, 10, 11, 12, 13, 14, or amino acids in length. In an embodiment, the SARS-CoV-2 protein is selected from the group consisting of orf1a/b, S protein, N protein, M protein, orf3a, and orf7a. In an embodiment, the immunogenic polypeptide is capable of eliciting a T cell response in a subject. In an embodiment, the immunogenic composition comprises more than one immunogenic polypeptide. In still another embodiment, the immunogenic composition further comprises an adjuvant. In yet another embodiment, the immunogenic composition is capable of eliciting a T cell response in a subject. In another embodiment, the administered immunogenic composition induces an immune response against the SARS-CoV-2 in the subject. In still another embodiment, the administered immunogenic composition induces a T cell immune response against the SARS-CoV-2 in the subject. In yet another embodiment, the T cell immune response is a CD8+ T cell immune response.

In yet another aspect, a method of identifying a peptide-binding molecule, or antigen-binding fragment thereof, that binds to a peptide epitope of at least one immunogenic polypeptide described herein comprising a) providing a cell presenting a peptide epitope of said at least one immunogenic polypeptide described herein in the context of a MHC molecule on the surface of the cell, optionally, wherein the cell comprises a nucleic acid encoding and expressing the at least one immunogenic polypeptide; b) determining binding of a plurality of candidate peptide-binding molecules or antigen-binding fragments thereof to the peptide epitope in the context of the MHC molecule on the cell; and c) identifying one or more peptide-binding molecules or antigen-binding fragments thereof that bind to the peptide epitope in the context of the MHC molecule, is provided.

Numerous embodiments are further provided that may be applied to any aspect of the present invention and/or combined with any other embodiment described herein. For example, in one embodiment, the step a) comprises contacting the MHC molecule on the surface of the cell with a peptide epitope selected from Table 1A, 1B, 1C, 1D, 1E, and/or 1F. In another embodiment, the step a) comprises transfecting the cell with a nucleic acid encoding an immunogenic polypeptide described herein, either as a basic nucleic acid encoding an immunogenic polypeptide described herein or as a vector comprising such a basic nucleic acid, such as comprising a heterologous sequence encoding a peptide epitope selected from Table 1A, 1B, 1C, 1D, 1E, and/or 1F.

In another aspect, a method of identifying a peptide-binding molecule or antigen-binding fragment thereof that binds to a peptide epitope of at least one immunogenic polypeptide described herein, comprising a) providing a stable MHC-peptide complex comprising a peptide epitope of said at least one immunogenic polypeptide described herein in the context of an MHC molecule; b) determining binding of a plurality of candidate peptide-binding molecules or antigen-binding fragments thereof to the stable MHC-peptide complex; and c) identifying one or more peptide-binding molecules or antigen-binding fragments thereof that bind to the stable MHC-peptide complex, is provided.

Numerous embodiments are further provided that may be applied to any aspect of the present invention and/or combined with any other embodiment described herein. For example, in one embodiment, the MHC molecule is a MHC multimer, optionally wherein the MHC multimer is a tetramer. In another embodiment, the MHC molecule is a MHC class I molecule. In still another embodiment, the MHC molecule comprises an MHC alpha chain that is an HLA serotype selected from the group consisting of HLA-A*02, HLA-A*03, HLA-A*01, HLA-A*11, HLA-A*24, and HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. In yet another embodiment, the peptide epitope and the MHC molecule are covalently linked and/or wherein the alpha and beta chains of the MHC molecule are covalently linked. In another embodiment, the stable MHC-peptide complex comprises a detectable label, optionally wherein the detectable label is a fluorophore. In still another embodiment, the plurality of candidate peptide binding molecules comprises one or more T cell receptors (TCRs), or one or more antigen-binding fragments of a TCR. In yet another embodiment, the plurality of candidate peptide binding molecules comprises at least 2, 5, 10, 100, 10³, 10⁴, 10⁵, 10⁶, 10⁷, 10⁸, 10⁹, or more, different candidate peptide binding molecules. In another embodiment, the plurality of candidate peptide binding molecules comprises one or more candidate peptide binding molecules that are obtained from a sample from a subject or a population of subjects; or the plurality of candidate peptide binding molecules comprises one or more candidate peptide binding molecules that comprise mutations in a parent scaffold peptide binding molecule obtained from a sample from a subject. In still another embodiment, the subject or population of subjects are a) not infected with SARS-CoV-2 and/or have recovered from COVID-19 orb) infected with SARS-CoV-2 and/or have COVID-19. In yet another embodiment, the subject or population of subjects has been vaccinated with one or more immunogenic polypeptides, wherein the immunogenic polypeptides comprise a peptide epitope selected from Table 1A, 1B, 1C, 1D, 1E, and/or 1F. In another embodiment, the subject is a mammal, optionally wherein the mammal is a human, a primate, or a rodent. In still another embodiment, the subject is an HLA-transgenic mouse and/or is a human TCR transgenic mouse. In yet another embodiment, the sample comprises T cells. In another embodiment, the sample comprises peripheral blood mononuclear cells (PBMCs) or CD8+ memory T cells. In still another embodiment, the antigen-binding fragment of a TCR is a single chain TCR (scTCR).

In another aspect, the peptide-binding molecule or antigen-binding fragment thereof identified according to a method described herein, is provided, optionally wherein the binding moiety is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

In still another aspect, a method of treating SARS-CoV-2 infection in a subject comprising administering to the subject a therapeutically effective amount of genetically engineered T cells that express a TCR identified by a method described herein, is provided.

In yet another aspect, a method of treating SARS-CoV-2 infection in a subject comprising administering to the subject a therapeutically effective amount of genetically engineered T cells that express a TCR that binds to a peptide epitope of at least one immunogenic polypeptide described herein, is provided.

In another aspect, a method of treating SARS-CoV-2 infection in a subject comprising administering to the subject a therapeutically effective amount of genetically engineered T cells that express a TCR that binds to a stable MHC-peptide complex comprising a peptide epitope of at least one immunogenic polypeptide described herein in the context of an MHC molecule, is provided.

Numerous embodiments are further provided that may be applied to any aspect of the present invention and/or combined with any other embodiment described herein. For example, in one embodiment, the MHC molecule is a MHC multimer, optionally wherein the MHC multimer is a tetramer. In another embodiment, the MHC molecule is a MHC class I molecule. In still another embodiment, the MHC molecule comprises an MHC alpha chain that is an HLA serotype selected from the group consisting of HLA-A*02, HLA-A*03, HLA-A*01, HLA-A*11, HLA-A*24, and HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. In yet another embodiment, the peptide epitope and the MHC molecule are covalently linked and/or wherein the alpha and beta chains of the MHC molecule are covalently linked. In another embodiment, the stable MHC-peptide complex comprises a detectable label, optionally wherein the detectable label is a fluorophore. In still another embodiment, the T cells are isolated from a) the subject, b) a donor not infected with SARS-CoV-2, or c) a donor recovered from COVID-19.

In still another aspect, a method of preventing and/or treating SARS-CoV-2 infection in a subject comprising transfusing antigen-specific T cells to the subject, wherein the antigen-specific T cells are generated by a) stimulating PBMCs or T cells from a subject with an immunogenic polypeptide described herein, a nucleic acid encoding an immunogenic polypeptide described herein, a stable MHC-peptide complex comprising a peptide epitope of at least one immunogenic polypeptide described herein, or a cell that encodes and/or presents a peptide of at least one immunogenic polypeptide described herein in the context of a MHC molecule on its cell surface, such as from an immunogenic polypeptide expressing construct described herein; and b) expanding antigen-specific T cells in vitro, optionally isolating PBMCs or T cells from the subject before stimulating the PBMCs or T cells, is provided.

Numerous embodiments are further provided that may be applied to any aspect of the present invention and/or combined with any other embodiment described herein. For example, in one embodiment, the T cell is a naive T cell, a central memory T cell, or an effector memory T cell. In another embodiment, the T cell is a CD8+ memory T cell. In still another embodiment, the agents are placed in contact under conditions and for a time suitable for the formation of at least one immune complex between the peptide epitope, immunogenic polypeptide, stable MHC-peptide complex, T cell receptor, and/or T cell. In yet another embodiment, the peptide epitope, immunogenic polypeptide, stable MHC-peptide complex, and/or T cell receptor are expressed by cells and the cells are expanded and/or isolated during one or more steps. In another embodiment, the subject is a mammal, optionally wherein the mammal is a human, a primate, or a rodent.

### BRIEF DESCRIPTION OF THE DRAWINGS

The patent of application file contains at least one drawing executed in color. Copies of this patent or patent application publication with color drawing(s) will be provided by the Office upon request and payment of the necessary fee.

FIG. 1 shows representative polyepitope vaccine constructs (e.g., embodiments of an immunogenic polypeptide) encoding immunodominant epitopes.

FIG. 2 shows representative next-generation SARS-CoV-2 vaccine constructs (e.g., embodiments of immunogenic polypeptides).

FIG. 3 shows responses of COVID-19 patient memory CD8 T cells to various vaccine constructs. The data are the mean of two patients (shown as individual dots).

FIG. 4 shows that select vaccine constructs show superior CD8 T cell activation compared to S protein alone. Memory CD8 T cells from 9 patients were tested for reactivity to the SARS-CoV-2 vaccine constructs to generate the data.

FIG. 5A-FIG. 5C show representative 19-epitope constructs. FIG. 5A shows a representative 19-epitope construct with 3aa linkers. FIG. 5B shows a representative 19-epitope construct with KAA linkers. FIG. 5C shows a representative 19-epitope construct with no linkers.

FIG. 6A-FIG. 6C show representative 27-epitope constructs. FIG. 6A shows a representative 27-epitope construct with 3aa linkers. FIG. 6B shows a representative 27-epitope construct with KAA linkers. FIG. 6C shows a representative 27-epitope construct with no linkers.

FIG. 7 shows a representative fragment construct (e.g., an embodiment of at least two peptide fragments each of which comprises at least two peptide epitopes).

FIG. 8 shows a representative fragment construct fused to an S protein segment and P2A.

FIG. 9A-FIG. 9E show representative polyepitope construct fusions. FIG. 9A shows a representative 19-epitope construct with 3aa linkers that is fused to an S protein segment and P2A. FIG. 9B shows a representative 27-epitope construct with 3aa linkers that is fused to an S protein segment and P2A. FIG. 9C shows a representative 19-epitope construct comprised within 14 fragments from the SARS-CoV-2 N, Orf1a, M, Orf3a, and S proteins. Each fragment is encoded by a nucleic acid sequences that ranges from 27 nucleotides to 195 nucleotides and contains one or more of the discovered epitopes covering a total of 19 known epitopes. The 14 fragment construct was designed to include any potential immunogenic epitopes that can be presented from the same proteasomal fragments as the discovered epitopes and to include potential undiscovered epitopes that can be presented on MHC molecules other than the MHC molecules known to bind and present the discovered epitopes. FIG. 9D shows a representative polypeptide construct comprised within fragments spanning the entire (full) SARS-CoV-2 M, N, and Orf3a proteins but represented in non-contiguous fragments, as well as an S protein fragment, in combination with 17 epitopes from the SARS-CoV-2 Orf1ab and S proteins. This construct is a combination of a polyepitope construct and a fragment-based construct. For example, the 5′ portion of the construct is a concatamer of 17 epitopes from the spike and orf1ab proteins of SARS-CoV-2 linked together by the surrounding 3 native amino acids. The 3′ portion of the construct is a concatamer of fragments that collectively span the N, Orf3a, and M proteins, and contain one fragment of the S protein. To avoid production of a functional protein (e.g., the Orf3a protein is known to have an immunosuppressive activity), the N, Orf3a, and M proteins were split into 2-3 fragments and dispersed in the sequence by alternating the order of each fragment. All 27 discovered epitopes are represented in this construct. The entire (full) N, Orf3a, and M regions of the SARS-CoV-2 genome were included because the N, Orf3a, and M proteins contained the highest abundance of epitopes when adjusted for the size of the region. Without being bound by theory, it is believed that this region has a biological driver causing the processing and presentation of immunogenic epitopes, and therefore are likely to contain epitopes that can be presented by many different MHCs potentially expanding the number of patients that can respond to the construct. The title, B.1.617.2_S.PP, in the figure refers to the PP-stabilized form of the S protein from the Delta variant (B.1.617.2). The fragments of the S protein included in this representative construct are from the Delta variant of SARS-CoV-2. FIG. 9E shows a representative polyepitope construct comprised within fragments spanning the entire (full) SARS-CoV-2 M, N, and Orf3a proteins but represented in non-contiguous fragments in combination with fragments from the SARS-CoV-2 ORF1ab and S proteins. The B.1.617.2_S.PP_EpiFrag-M/N/ORF3a contains the same 3′ N, Orf3a, M and S fragments as the Epi-M/N/ORF3a construct described in FIG. 9D, except that the 5′ 17epi fragment portion is replaced with fragments from the Orf1ab and S proteins that surround the 17 epitopes from FIG. 9D. They are the same fragments from the 14_fragment epitope. The title, B.1.617.2_S.PP, in the figure refers to the PP-stabilized form of the S protein from the Delta variant (B.1.617.2). The fragments of the S protein included in this representative construct are from the Delta variant of SARS-CoV-2. The representative sequences shown in FIG. 9C-FIG. 9E do not include additional sequences, such as linkers. For example, the 3AA linker is unnecessary because the larger fragment provides the space of the linker for the identified epitopes, and the unidentified epitopes are undefined such that the absence of a linker ensures that epitopes are not directly at the ends of the segments. The segments are placed directly without P2A sites. The one exception is the polyepitope portion of FIG. 9D that contains 3AA linkers.

FIG. 10 shows that memory T cell (Tmem) pools from SARS-CoV-2 patients recognize vaccine constructs of FIG. 9C. Fourteen (14) fragment mRNA constructs (described in FIG. 9C) were introduced with lipid nanoparticles to monoallelic HEK293T cells modified to express a single MHC-I molecule. Memory T cells isolated from recovered SARS-CoV-2 patients were co-cultured with construct treated mono-allelic HEK293T cells and reactivity to the constructs was tested by measuring interferon gamma release by ELISA.

FIG. 11 shows that TCRs that recognize specific epitopes of SARS-CoV-2 react to dendritic cells treated with LNPs containing the 27 polyepitope construct of FIG. 6A. Monocyte-derived dendritic cells (moDCs) were isolated from blood collected in 2019 in the United States before widespread infection of SARS-CoV2. moDCs were treated with LNPs containing mRNA of the constructs in FIG. 6A. T cells specific to each indicated epitope from Table 1A or 1F were co-cultured with the LNP treated moDCs and T cell reactivity was measured by flow cytometric staining of the activation induced markers (AIM) CD69 and CD137. Thus, the epitopes contained within these constructs are processed and presented sufficiently to elicit a response from epitope specific T cells.

FIG. 12 shows that TCRs that recognize epitopes of SARS-CoV-2 utilize common TRAV genes. Shown are the frequency of specific clones that recognize the YLQ, KLW, and SPR epitopes from Table 1A and F. Highlighted are the shared dominant TRAV genes. See also Ferretti et al. (2020) Immunity 53: 1095-1107, especially at FIG. 4 and the materials and methods for additional support demonstrating that common TRAV genes are utilized by TCRs.

FIG. 13 shows a representative, non-limiting example of an in vitro vaccine model.

FIG. 14 shows that a pulsed SARS-CoV-2 immunodominant peptide induces an expansion of peptide-specific T cells from the naïve T cell population in an in vitro vaccine model from FIG. 13. MHC-peptide tetramer staining was used to detect peptide specific TCRs as indicated.

FIG. 15 shows that a vaccine construct induces an expansion of peptide-specific T cells from the naïve T cell population in an in vitro vaccine model from FIG. 13.

FIG. 16A-FIG. 16C show that known V regions corresponding to epitopes known to be presented by the tested MHCs (FIG. 12) are dominant among top expanded TCR clones from the in vitro vaccine model treated with constructs of FIG. 9C, but not from untreated controls. FIG. 16A illustrates the TRAV genes of the expanded clones highlighting the known TRAV gene corresponding to TCRs known to bind the YLQ peptide with a background subtracted frequency above the maximum of the control sample. FIGS. 16B and 16C illustrate the TRAV genes of the expanded clones responding to the vaccine construct of FIG. 9C when restimulated with known peptides described in Table 1A and F. TRAV genes corresponding to TCRs known to bind peptides within the construct in FIG. 9C with a background subtracted frequency above the maximum of the control sample are highlighted for the indicated MHCs tested. Thus, these constructs induce a response from naïve T cells in an in vitro vaccine model that recapitulates the immune response to natural infection with SARS-CoV-2.

### DETAILED DESCRIPTION OF THE INVENTION

The present invention is based, at least in part, on the discovery of SARS-CoV-2 virus-specific immunogenic polypeptide constructs, which for example can be used as vaccines. A systematic, comprehensive survey was carried out to map the precise T cell targets recognized by convalescent COVID-19 patients. Strikingly, the study revealed a limited set of highly immunodominant peptide antigens that are recurrently recognized across patients, including several that appear to be universally recognized.

To inform the design of such vaccines having immunogenic peptides, a comprehensive, genome-wide analysis of the targets recognized by CD8 T cells following infection with SARS-CoV-2 was undertaken. The landscape of targets recognized on the six most common HLA alleles—HLA-A:02, HLA-A01, HLA-A03, HLA-A11, HLA-A24, and HLA-B07—a set where >85% of people globally express at least one of these alleles was mapped. A core set of immunodominant epitopes for each allele that is recurrently recognized by a majority of the patients with the appropriate HLA allele was identified and validated. These epitopes were revealed to be robustly presented on MHC molecules, efficiently recognized by the naïve CD8 T cell repertoire across patients, and likely to be protective (based on their outsized contribution to the overall CD8 T cell response, which is likely protective). In addition to identifying precise epitopes presented on particular HLA alleles, patterns of T cell reactivity to the SARS-CoV-2 genome were also revealed. Regions of the genome that are recurrently recognized across HLA alleles—including the N, M, and ORF3a proteins, and parts of ORF1ab—were found, and these regions represent attractive vaccine antigens that are likely to generate CD8 T cell responses restricted on additional alleles as well. CD8 T cell responses to specific segments of the SARS-CoV-2 genome that are cross-reactive with endemic betacoronaviruses were also identified. These regions are of particular interest since vaccines targeting them will be able to boost any pre-existing CD8 T cells reactive to the endemic betacoronaviruses and have the potential to generate more robust responses.

This data has enabled the design of vaccine candidates that contain a broad set of CD8 T cell antigens to more accurately recapitulate natural immunity to SARS-CoV-2. Specifically, the vaccines that were designed include poly-epitope vaccines and fragment-based vaccines.

In polyepitope vaccines, a number of immunodominant CD8 T cell epitopes were concatenated for expression as a single polyprotein. This approach has the advantage of providing maximal density of validated T cell epitopes. Variants ranging from 6-29 total epitopes were designed. A key design feature of polyepitope vaccines is the ability to maximize efficient processing and presentation of the desired immunodominant epitopes while minimizing any non-natural junctional epitopes. Polyepitopes containing optimal proteasomal cleavage sequences were also designed, either derived from the natural protein sequence (the amino acids directly surrounding each epitope in the full-length protein, which are validated to result in efficient presentation based on our screening data) or using synthetic proteasomal cleavage sequences as inter-epitope linkers. A bioinformatic approach to select the optimal epitope order that minimizes the generation of predicted high-affinity junctional epitopes (selecting an optimal order from millions of tested variants) was also used.

In fragment-based vaccines, a set of longer genomic segments from SARS-CoV-2 that were identified to be immunodominant across a range of HLA alleles were concatenated. This approach has the advantage of including longer stretches of SARS-CoV-2 protein sequence, which increases the likelihood of encoding additional CD8 T cell epitopes that are presented by other HLA alleles that we did not investigate. Between 2 and 6 segments were concatenated for these vaccines.

Both classes of vaccines are highly modular in terms of broader context and delivery. They can serve as stand-alone vaccines focused on boosting CD8 T cell responses or be co-introduced alongside antigens designed to generate neutralizing antibody responses. In the latter case, they can be expressed along with other antigens following a ribosomal restart site such as a P2A sequence. Several of the designs include the optimized S protein (containing two proline mutations designed to enhance the stability of the protein) followed by a P2A sequence and the polyepitope or polyfragment protein. They can be delivered using mRNA, DNA, viral vectors, or as purified protein.

As one example related to the particular epitopes of the constructs, it was determined herein that the CD8+ T cell response is dominated by a few (3-8) highly antigenic (immunodominant) epitopes in SARS-CoV-2 that are shared among patients with the same HLA type. These epitopes are largely unique to SARS-CoV-2 (i.e., do not occur in “common cold” coronaviruses), are invariant among viral isolates, and are frequently targeted by multiple clonotypes within each patient. At least twenty-nine shared epitopes were identified across the six HLA types studied. Notably, only ˜10% (3 of 29) of the epitopes occur in the S protein (i.e., ˜90% of SARS-CoV-2 immunodominant epitopes are located outside of the S protein), highlighting the need for new classes of vaccines that are designed to elicit a broader CD8+ T cell response. Indeed, none of the mutations in the UK, South African, Brazilian, or Delta variants occurs in these 29 epitopes and some HLA types did not yield any spike protein epitopes when analyzing patients infected with SARS-CoV-2 (e.g., screening data for five A*01:01 patients and five A*11:01 patients did not identify any epitopes in spike protein). Remarkably, it was determined that 94% of screened patients had T cells that recognized at least one of the three most dominant epitopes for a given HLA and 53% of patients had T cells that recognized all three of the most dominant epitopes for a given HLA. Additional confirmatory analyses in 18 additional A*02:01 patients reiterated the presence of memory CD8+ T cells specific for the top six identified A*02:01 epitopes, and single-cell sequencing revealed that patients often have >5 different T cell clones targeting each epitope, but that the same T cell receptor Va and Vb regions are predominantly used to recognize these epitopes, even across patients. T cells that target most of these immunodominant epitopes (27 of 29) do not cross-react with the endemic coronaviruses that cause the common cold, and the epitopes do not occur in regions with high mutational variation. These results provide useful tools to better understand the CD8+ T cell response in COVID-19 and have significant implications for vaccine design and development.

Accordingly, the present invention relates, in part, to the identified immunogenic polypeptide constructs, compositions comprising these immunogenic polypeptide constructs alone or with MHC molecules, stable MHC-peptide complexes, methods of diagnosing, prognosing, and monitoring T cell response to SARS-CoV-2, and methods for preventing and/or treating SARS-CoV-2 infection by administering immunogenic compositions comprising and/or encoding the identified immunogenic polypeptide constructs.

### I. Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

Conventional T cells, also known as Tconv or Teffs, have effector functions (e.g., cytokine secretion, cytotoxic activity, anti-self-recognition, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8+ cytotoxic T lymphocytes. As described further herein, cytotoxic T cells are CD8+ T lymphocytes. “Naïve Tcons” are CD4⁺ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

The term “vaccine” refers to a pharmaceutical composition that elicits an immune response to an antigen of interest. The vaccine may also confer protective immunity upon a subject.

“Vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the present invention is intended to include such other forms of expression vectors which serve equivalent functions and which become subsequently known in the art.

The term “immunotherapeutic agent” may include any molecule, peptide, antibody or other agent which can stimulate a host immune system to generate an immune response to a viral infection in the subject. Various immunotherapeutic agents are useful in the compositions and methods described herein.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM, IgG1, IgG2C, and the like) that is encoded by heavy chain constant region genes.

As used herein, the term “K_{D}” is intended to refer to the dissociation equilibrium constant of a particular antibody-antigen interaction. The binding affinity of antibodies encompassed by the present invention may be measured or determined by standard antibody-antigen assays, for example, competitive assays, saturation assays, or standard immunoassays such as ELISA or RIA.

A “kit” is any manufacture (e.g., a package or container) comprising at least one reagent, e.g., a probe or small molecule, for specifically detecting and/or affecting the expression of a marker encompassed by the present invention. The kit may be promoted, distributed, or sold as a unit for performing the methods encompassed by the present invention. The kit may comprise one or more reagents necessary to express a composition useful in the methods encompassed by the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit may be included.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of a viral infection or the likelihood of recovery from the disease. In some embodiments, the use of statistical algorithms provides a prognosis of a viral infection in an individual. For example, the prognosis may be surgery, development of a clinical subtype of a viral infection, development of one or more clinical factors, or recovery from the disease.

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method encompassed by the present invention further comprises obtaining the sample from the individual prior to detecting or determining the presence or level of at least one marker in the sample.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which may be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “subject” refers to any healthy animal, mammal or human, or any animal, mammal or human afflicted with a viral infection, e.g., SARS-CoV-2 infection. The term “subject” is interchangeable with “patient.”

As used herein, “percent identity” between amino acid sequences is synonymous with “percent homology,” which can be determined using the algorithm of Karlin and Altschul (Proc. Natl. Acad. Sci. USA 87, 2264-2268, 1990), modified by Karlin and Altschul (Proc. Natl. Acad. Sci. USA 90, 5873-5877, 1993). The noted algorithm is incorporated into the NBLAST and XBLAST programs of Altschul et al. (J. Mol. Biol. 215, 403-410, 1990). BLAST nucleotide searches are performed with the NBLAST program, score=100, wordlength=12, to obtain nucleotide sequences homologous to a polynucleotide described herein. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (Nucleic Acids Res. 25, 3389-3402, 1997). When utilizing BLAST and Gapped BLAST programs, the default parameters of the respective programs (e.g., XBLAST and NBLAST) are used.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

A “transcribed polynucleotide” or “nucleotide transcript” is a polynucleotide (e.g., an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g., splicing), if any, of the RNA transcript, and reverse transcription of the RNA transcript.

The term “T cell” includes CD4⁺ T cells and CD8⁺ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “T cell receptor” or “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR may contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods encompassed by the present invention may be administered in a sufficient amount to produce a reasonable benefit/risk ratio applicable to such treatment.

The terms “therapeutically-effective amount” and “effective amount” as used herein means that amount of a compound, material, or composition comprising a compound encompassed by the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, similarly, the IC₅₀ may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, T cell immune response in an assay may be increased by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a viral load may be achieved.

“Treating” a disease in a subject or “treating” a subject having a disease refers to subjecting the subject to a pharmaceutical treatment, e.g., the administration of a drug, such that at least one symptom of the disease is decreased or prevented from worsening.

The term “body fluid” refers to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal may result in cytokine secretion, e.g., in a T cell that has received a T cell-receptor-mediated signal. Immune cells that have received a cell-receptor mediated signal, e.g., via an activating receptor are referred to herein as “activated immune cells.”

The term “determining a suitable treatment regimen for the subject” is taken to mean the determination of a treatment regimen (i.e., a single therapy or a combination of different therapies that are used for the prevention and/or treatment of the viral infection in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “adjuvant” as used herein refers to substances, which when administered prior, together or after administration of an antigen accelerates, prolong and/or enhances the quality and/or strength of an immune response to the antigen in comparison to the administration of the antigen alone. Adjuvants can increase the magnitude and duration of the immune response induced by vaccination.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

The term “SARS-CoV-2” or “Severe Acute Respiratory Syndrome Coronavirus 2” refers to the causative agent of coronavirus disease 2019 (COVID-19). SARS-CoV-2 was identified as a pandemic by the World Health Organization (WHO) on Mar. 11, 2020. In supporting the process of entry of the virus into the host cell, SARS-CoV2 binds to the ACE2 receiver that is highly expressed in the lower respiratory tract such as type II alveolar cells (AT2) of the lungs, upper esophagus and stratified epithelial cells, and other cells such as absorptive enterocytes from the ileum and colon, cholangiocytes, myocardial cells, kidney proximal tubule cells, and bladder urothelial cells. Therefore, patients who are infected with this virus not only experience respiratory problems such as pneumonia leading to Acute Respiratory Distress Syndrome (ARDS), but also experience disorders of heart, kidneys, and digestive tract.

There is no specific treatment for eradication of the SARS-CoV2 virus in patients. Therapeutic approaches for another 0-coronavirus approach such as SARS-CoV or MERS-CoV treatments may be used. Some of these approaches including lopinavir/ritonavir, chloroquine, and hydroxychloroquine. Aerosol inhalation of interferon α twice per night also could be used. In some cases, combinations of interferon-α combined with ribavirin have commonly used coronaviruses (such as MERS-CoV). It was also found that the combination of interferon with steroid drugs can accelerate lung repair and increase oxygen survival levels. However, inconsistent results have been shown for therapy using interferon α.

SARS-CoV-2 virus is an enveloped, non-segmented, positive sense RNA virus that is included in the sarbecovirus, ortho corona virinae subfamily which is broadly distributed in humans and other mammals. Its diameter is about 65-125 nm, containing single strands of RNA and provided with crown-like spikes on the outer surface. SARS-CoV2 is a novel β-coronavirus after the previously identified SARS-CoV and MERS-CoV which led to pulmonary failure and potentially fatal respiratory tract infection and caused outbreaks mainly in Guandong, China and Saudi Arabia.

The genome size of the SARS-CoV-2 varies from 29.8 kb to 29.9 kb and its genome structure followed the specific gene characteristics to known CoVs. The 5′ more than two-thirds of the genome comprises orf1a/b encoding orf1a/b polyproteins, while the 3′ one third consists of genes encoding four main structural proteins including spike (S) glycoprotein, small envelope (E) glycoprotein, membrane (M) glycoprotein, and nucleocapsid (N) protein. Additionally, the SARS-CoV-2 contains 6 accessory proteins, encoded by ORF3a, ORF6, ORF7a, ORF7b, and ORF8 genes (Khailany et al. (2020) Gene Rep 19:100682).

The ORF1ab gene is the largest gene segment of the coronavirus and it constitutes two ORF, i.e., ORF1a and ORF1b, to produce two large overlapping polyproteins, pp1a (orf1a polyprotein) and pp1ab (orf1ab polyprotein) by contributing a ribosomal frame shifting event. The polyproteins are supplemented by protease enzymes namely papain-like proteases (PLpro) and a serine type Mpro (chymotrypsin-like protease (3CLpro)) protease that are encoded in nsp3 and nsp 5. Subsequently, cleavage occurs between pp1a and pplab into nonstructural proteins (nsps) 1-11 and 1-16, respectively. The nsps play an important role in many processes in viruses and host cells. Representative sequences of orf1a polyprotein and orf1ab polyprotein are presented below in Table 1K.

ORF3a is one of the accessory proteins encoded by SARS-CoV-2 genome. Recent studies have showed that the functional domains of SARS-CoV-2 ORF3a protein are linked to virulence, infectivity, ion channel formation, and virus release (Issa et al. (2020) mSystems 5:e00266-20). Representative sequences of ORF3a are presented below in Table 1K.

ORF7a is another SARS-CoV-2 genome-encoded accessory protein that is composed of a type I transmembrane protein that localizes primarily to the Golgi apparatus but can be found on the cell surface. SARS-CoV ORF7a overlaps ORF7b in the viral genome, where they share a transcriptional regulatory sequence (TRS). In some embodiments, ORF7a has a 15-amino-acid (aa) N-terminal signal peptide, an 81-aa luminal domain, a 21-aa transmembrane domain, and a 5-aa cytoplasmic tail (Taylor et al. (2015) J. Virol. 89:11820-11833). Representative sequences of ORF7a are presented below in Table 1K.

The spike or S glycoprotein is a transmembrane protein with a molecular weight of about 150 kDa found in the outer portion of the virus. S protein has an RBD located in the 51 subunit of the virus that facilitates entry of the virus into the host cell by binding to its receptors on the host cell, ACE2. S protein forms homotrimers protruding in the viral surface and facilitates binding of envelope viruses to host cells by attraction with angiotensin-converting enzyme 2 (ACE2) expressed in lower respiratory tract cells. This glycoprotein is cleaved by the host cell furin-like protease into 2 sub units namely 51 and S2. Part 51 is responsible for the determination of the host virus range and cellular tropism with the receptor binding domain make-up while S2 functions to mediate virus fusion in transmitting host cells. Representative sequences of S glycoprotein are presented below in Table 1K.

The nucleocapsid known as N protein is the structural component of CoV localizing in the endoplasmic reticulum-Golgi region that structurally is bound to the nucleic acid material of the virus. Because the protein is bound to RNA, the protein is involved in processes related to the viral genome, the viral replication cycle, and the cellular response of host cells to viral infections. N protein is also heavily phosphorylated and suggested to lead to structural changes enhancing the affinity for viral RNA. Representative sequences of N glycoprotein are presented below in Table 1K.

Another important part of this virus is the membrane or M protein, which is the most structurally structured protein and plays a role in determining the shape of the virus envelope. This protein can bind to all other structural proteins. Binding with M protein helps to stabilize nucleocapsids or N proteins and promotes completion of viral assembly by stabilizing N protein-RNA complex, inside the internal virion. Representative sequences of M protein are presented below in Table 1K.

The last component is the envelope or E protein which is the smallest protein in the SARS-CoV-2 structure that plays a role in the production and maturation of this virus.

The genomic information of SARS-CoV-2 is publicly available and can be obtained from, for example, the NCBI Severe acute respiratory syndrome coronavirus 2 database (available on the World Wide Web at ncbi.nlm.nih.gov/sars-cov-2/) and NGDC Genome Warehouse (available at bigd.big.ac.cn/gwh/), together with epidemiological data for the sequenced isolates. There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) may be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### II. Peptides & Constructs

In certain aspects, provided herein are methods and compositions for the treatment and/or prevention of COVD-19 through the induction of an immune response against SARS-CoV-2 through the administration of identified SARS-COV-2 immunodominant peptides or nucleic acids encoding identified SARS-COV-2 immunodominant peptides.

In some aspects, provided herein are immunogenic polypeptides comprising at least two peptide epitopes or at least two peptide fragments each of which comprises at least two peptide epitopes. Various embodiments related to the peptide epitopes of these immunogenic polypeptides are described below (e.g., peptide epitopes described with respect to immunodominant peptides can also be the constituent peptide epitopes of the immunogenic polypeptides).

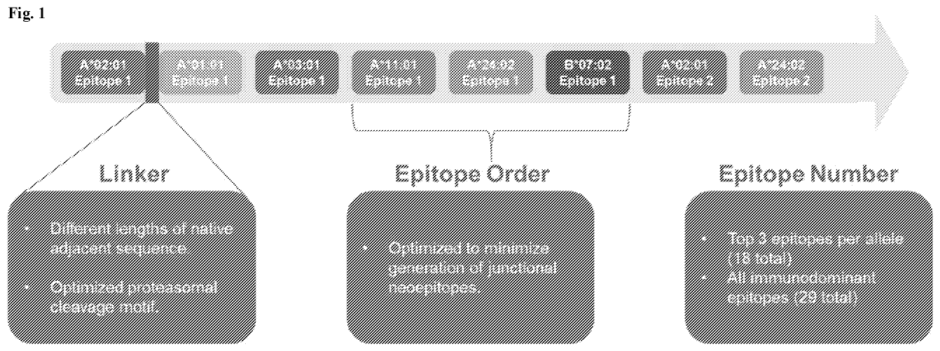

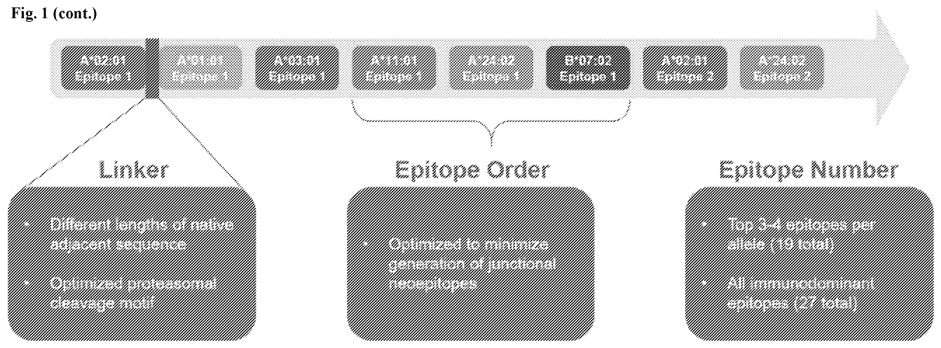

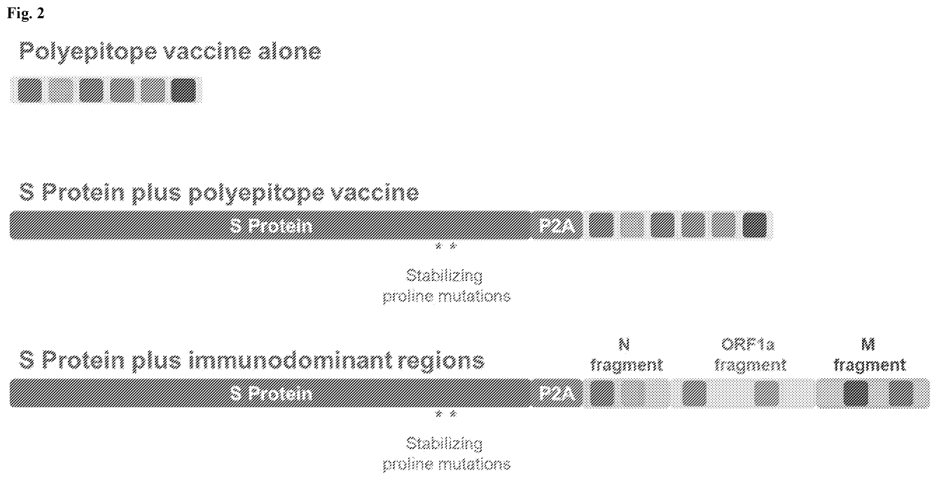

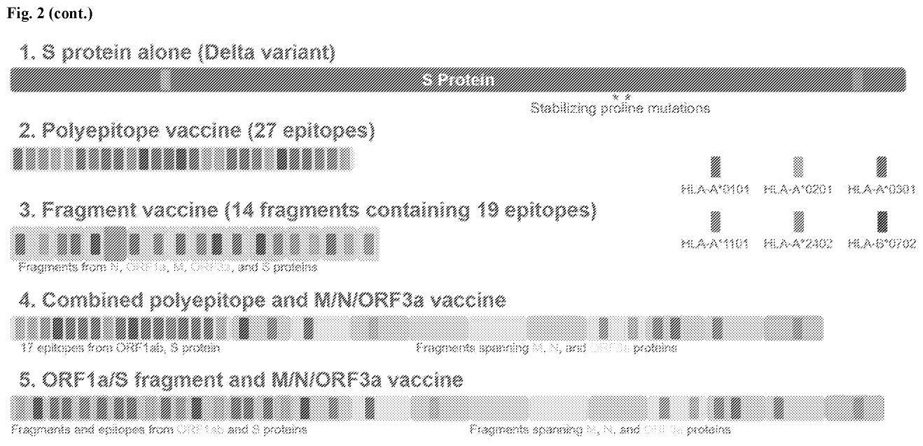

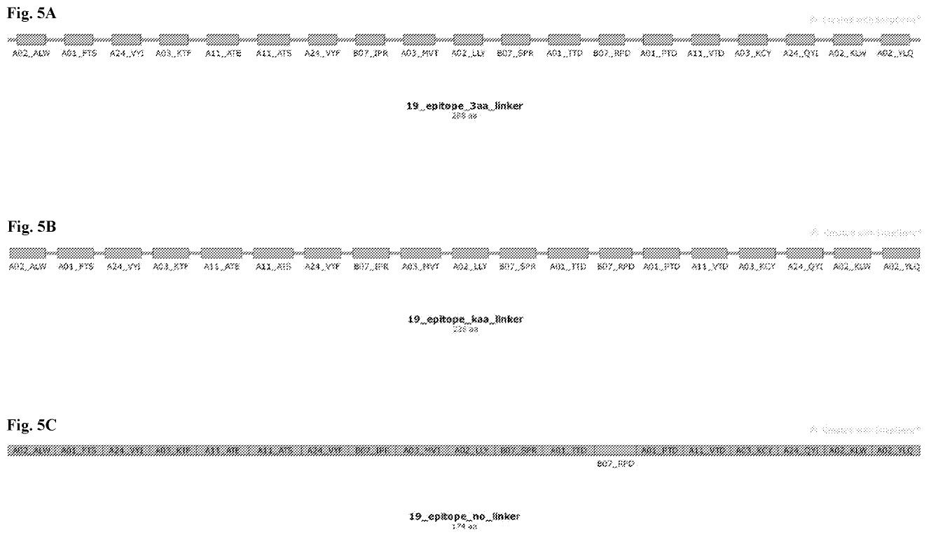

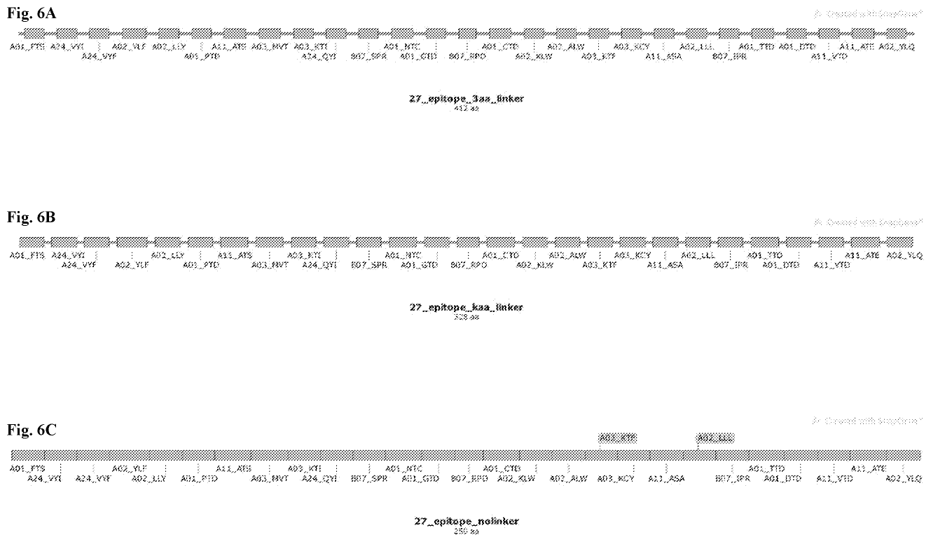

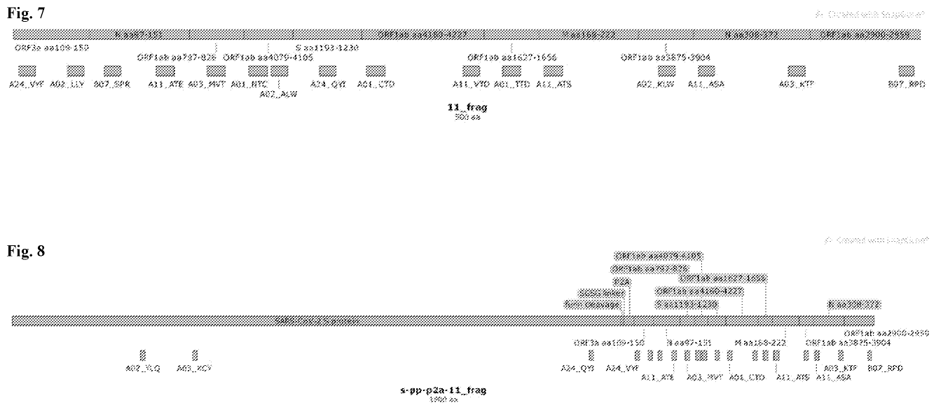

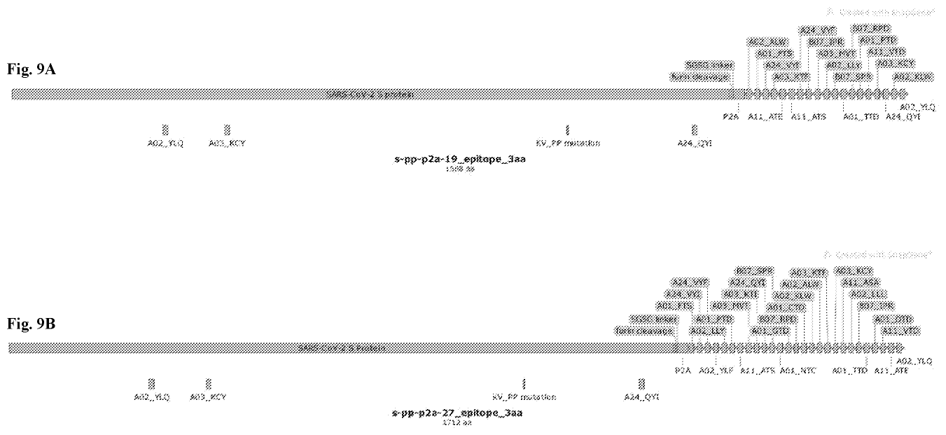

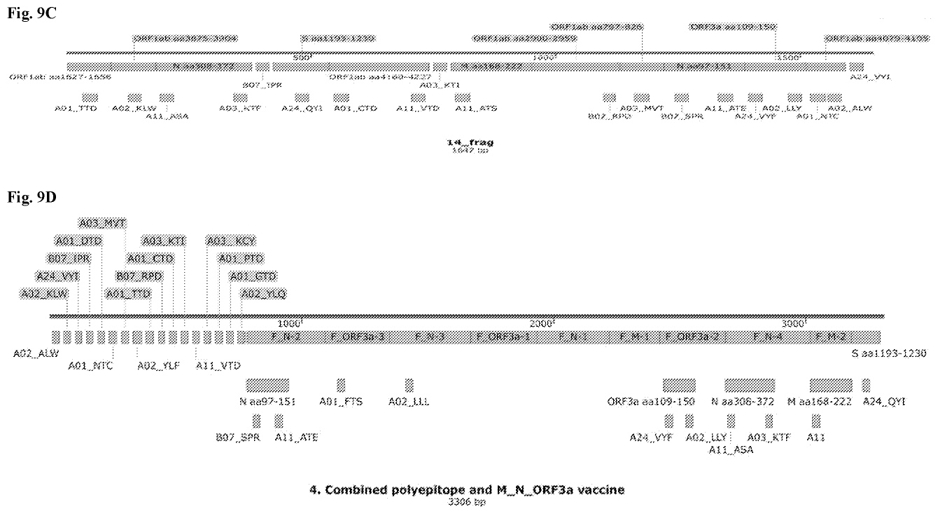

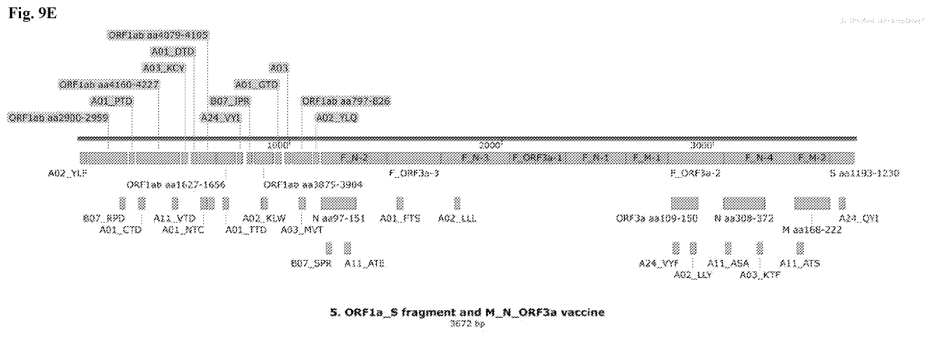

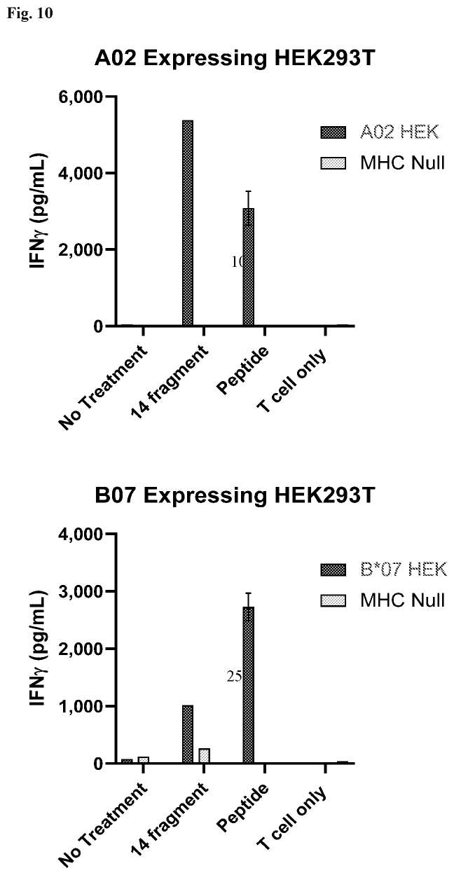

Certain exemplary immunogenic polypeptides (e.g., constructs) are shown in FIG. 1, FIG. 2, FIG. 5A-FIG. 5C, FIG. 6A-FIG. 6C, FIG. 7, FIG. 8, FIG. 9A, and FIG. 9B. As seen, the immunogenic polypeptides can include multiple (e.g., 4, 5, 6, 7, 8, 9, 10, 11, 12, 13, 14, 15, 16, 17, 18, 19, 20, 21, 22, 23, 24, 25, 26, 27, 28, 29) peptide epitopes (e.g., those from Tables 1A to 1F), can include fragments of SARS-CoV-2 proteins having at least two such epitopes, and can further have additional segments, such as an S protein segment and/or a ribosomal stop/restart segment, IRES segment, and/or a post-translational cleavage segment, optionally wherein the post-translational cleavage segment is a P2A segment. In some embodiments, the fragments of SARS-CoV-2 proteins comprising epitope(s) of interest can be defined by size, such as between 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 1, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 419, 420, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1235, 1240, 1245, 1250, 1255, 1260, 1265, 1270, 1271 amino acids (AA), or any range in between, inclusive, such as 8-1271, 30-40, 15-30, 15-40, 30-112, 8-38, 8-96, 8-104, 8-107, 8-112, 8-88, 8-87, 8-85, 8-66, 8-55, 222-275, 222-419, 275-419, 222-1271, and the like. In some embodiments, the smallest fragment is an epitope at 8 AA. In some embodiments, representative 14 fragment vaccine constructs for non-epitope fragments range from 30-40AA and the smallest fragment in 14 fragment=[3+9+3] epitope [3aa+epitope+3aa]). In some embodiments, the largest fragment of protein spanning fragments is 112 AA. In some embodiments, the S protein (1271 AA is covered by a fragment of 38 amino acids. In some embodiments, to N protein (419 AA) is covered by one or more fragments of 96 AA, 104 AA, and/or 107 AA (e.g., N_Frag1=96 AA, N_Frag2=104 AA and/or N_Frag3=107 AA). In some embodiments, the Orf3a protein (275 AA) is covered by one or more fragments of 85 AA, 87 AA, and/or 88 AA (e.g., 3a_Frag1=88 AA, 3a_Frag2=87 AA, and/or 3a_Frag 3=85 AA). In some embodiments, the M protein (222 AA) is covered by one or more fragments of 55 AA and/or 66 AA (e.g., M_Frag1=66 and/or M_Frag2=55).

In certain embodiments, the SARS-COV-2 immunodominant peptide comprises (e.g., consists of) a peptide epitope selected from Table 1A, 1B, 1C, 1D, 1E, and/or 1F. Peptide epitopes described herein may be combined with MHC molecules, such as particular HLA molecules having particular alpha chain alleles. For example, Table 1A peptides were identified in association with an MHC whose alpha chain had an HLA-A*02 serotype, such as that encoded by an HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, and/or HLA-A*0274 allele; Table 1C peptides were identified in association with an MHC whose alpha chain had an HLA-A*03 serotype, such as that encoded by an HLA-A*0301, HLA-A*0302, HLA-A*0305, and/or HLA-A*0307; Table 1B peptides were identified in association with an MHC whose alpha chain had an HLA-A*01 serotype, such as that encoded by an HLA-A*0101, HLA-A*0102, HLA-A*0103, and/or HLA-A*0116 allele; Table 1D peptides were identified in association with an MHC whose alpha chain had an HLA-A*11 serotype, such as that encoded by an HLA-A*1101, HLA-A*1102, HLA-A*1103, HLA-A*1104, HLA-A*1105, and/or HLA-A*1119 allele; Table 1E peptides were identified in association with an MHC whose alpha chain had an HLA-A*24 serotype, such as that encoded by an HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, and/or HLA-A*2458 allele; and Table 1F peptides were identified in association with an MHC whose alpha chain had an HLA-B*07 serotype, such as that encoded by an HLA-B*0702, HLA-B*0704, HLA-B*0705, HLA-B*0709, HLA-B*0710, HLA-B*0715, and/or HLA-B*0721 allele, as described further in the working examples. In some embodiments, the SARS-COV-2 immunodominant peptides are derived from a SARS-COV-2 protein selected from Table 1K. In some embodiments, one or more SARS-COV-2 immunodominant peptides are administered alone or in combination with an adjuvant.

In certain aspects, provided herein are compositions comprising one or more SARS-CoV-2 immunogenic peptides described herein and an adjuvant.

TABLE 1A
(HLA-A02)

| Peptide | Derived From SARS-COV-2 |
|---|---|
| Epitopes | Protein |
| KLWAQCVQL | ORF1ab |
| YLQPRTFLL | S |
| LLYDANYFL | ORF3a |
| ALWEIQQVV | ORF1ab |
| LLLDRLNQL | N |
| YLFDESGEFKL | ORF1ab |

TABLE 1B
(HLA-A01)

| Peptide | Derived From |
|---|---|
| Epitopes | SARS-COV-2 Protein |
| FTSDYYQLY | ORF3a |
| TTDPSFLGRY | ORF1ab |
| PTDNYITTY | ORF1ab |
| ATSRTLSYY | M |
| CTDDNALAYY | ORF1ab |
| NTCDGTTFTY | ORF1ab |
| DTDFVNEFY | ORF1ab |
| GTDLEGNFY | ORF1ab |

TABLE 1C
(HLA-A03)

| | Derived From |
|---|---|
| Peptide | SARS-COV-2 |
| Epitopes | Protein |
| KTFPPTEPK | N |
| KCYGVSPTK | S |
| MVTNNTFTLK | ORF1ab |
| KTIQPRVEK | ORF1ab |

TABLE 1D
(HLA-A11)

| | Derived From |
|---|---|
| Peptide | SARS-COV-2 |
| Epitopes | Protein |
| KTFPPTEPK | N |
| VTDTPKGPK | ORF1ab |
| ATEGALNTPK | N |
| ASAFFGMSR | N |
| ATSRTLSYYK | M |

TABLE 1E
(HLA-A24)

| | Derived From |
|---|---|
| Peptide | SARS-COV-2 |
| Epitopes | Protein |
| QYIKWPWYI | S |
| VYFLQSINF | ORF3a |
| VYIGDPAQL | ORF1ab |

TABLE 1F
(HLA-B07)

| | Derived From |
|---|---|
| Peptide | SARS-COV-2 |
| Epitopes | Protein |
| SPRWYFYYL | N |
| RPDTRYVL | ORF1ab |
| IPRRNVATL | ORF1ab |

TABLE 1G
>19_epitope_3aa_linker (288 aa)
YASALWEIQQVVDADHSYFTSDYYQLYSTQKHYVYIGDPA
QLPAPDAYKTFPPTEPKKDKIWVATEGALNTPKDHIITVA
TSRTLSYYKLGAYALVYFLQSINFVRISLEIPRRNVATLQ
AEPNMMVTNNTFTLKGGAKNPLLYDANYFLCWHKDLSPRW
YFYYLGTGYYHTTDPSFLGRYMSAESLRPDTRYVLMDGRK
VPTDNYITTYPGQCRFVTDTPKGPKVKYSTFKCYGVSPTK
LNDKYEQYIKWPWYIWLGSSSKLWAQCVQLHNDYVGYLQP
RTFLLKYN
>19_epitope_kaa linker (228 aa)
ALWEIQQVVKAAFTSDYYQLYKAAVYIGDPAQLKAAKTFP
PTEPKKAAATEGALNTPKKAAATSRTLSYYKKAAVYFLQS
INFKAAIPRRNVATLKAAMVTNNTFTLKKAALLYDANYFL
KAASPRWYFYYLKAATTDPSFLGRYKAARPDTRYVLKAAP
TDNYITTYKAAVTDTPKGPKKAAKCYGVSPTKKAAQYIKW
PWYIKAAKLWAQCVQLKAAYLQPRTFLL
>19_epitope_no_linker (174 aa)
ALWEIQQVVFTSDYYQLYVYIGDPAQLKTFPPTEPKATEG
ALNTPKATSRTLSYYKVYFLQSINFIPRRNVATLMVTNNT
FTLKLLYDANYFLSPRWYFYYLTTDPSFLGRYRPDTRYVL
PTDNYITTYVTDTPKGPKKCYGVSPTKQYIKWPWYIKLWA
QCVQLYLQPRTFLL
>27_epitope_3aa linker (412 aa)
HSYFTSDYYQLYSTQKHYVYIGDPAQLPAPYALVYFLQSI
NFVRIATYYLFDESGEFKLASHKNPLLYDANYFLCWHRKV
PTDNYITTYPGQITVATSRTLSYYKLGAPNMMVTNNTFTL
KGGASIIKTIQPRVEKKKLKYEQYIKWPWYIWLGKDLSPR
WYFYYLGTGTYKNTCDGTTFTYASAVHAGTDLEGNFYGPF
ESLRPDTRYVLMDGQTACTDDNALAYYNTTSSSKLWAQCV
QLHNDYASALWEIQQVVDADDAYKTFPPTEPKKDKSTFKC
YGVSPTKLNDAPSASAFFGMSRIGMLALLLLDRLNQLESK
SLEIPRRNVATLQAEYYHTTDPSFLGRYMSARDVDTDFVN
EFYAYLCRFVTDTPKGPKVKYIWVATEGALNTPKDHIYVG
YLQPRTFLLKYN
>27_epitope_kaa_linker (328 aa)
FTSDYYQLYKAAVYIGDPAQLKAAVYFLQSINFKAAYLFD
ESGEFKLKAALLYDANYFLKAAPTDNYITTYKAAATSRTL
SYYKKAAMVTNNTFTLKKAAKTIQPRVEKKAAQYIKWPWY
IKAASPRWYFYYLKAANTCDGTTFTYKAAGTDLEGNFYKA
ARPDTRYVLKAACTDDNALAYYKAAKLWAQCVQLKAAALW
EIQQVVKAAKTFPPTEPKKAAKCYGVSPTKKAAASAFFGM
SRKAALLLDRLNQLKAAIPRRNVATLKAATTDPSFLGRYK
AADTDEVNEFYKAAVTDTPKGPKKAAATEGALNTPKKAAY
LQPRTFLL
>27_epitope_nolinker (250 aa)
FTSDYYQLYVYIGDPAQLVYFLQSINFYLFDESGEFKLLL
YDANYFLPTDNYITTYATSRTLSYYKMVTNNTFTLKKTIQ
PRVEKQYIKWPWYISPRWYFYYLNTCDGTTFTYGTDLEGN
FYRPDTRYVLCTDDNALAYYKLWAQCVQLALWEIQQVVKT
FPPTEPKKCYGVSPTKASAFFGMSRLLLDRLNQLIPRRNV
ATLTTDPSFLGRYDTDFVNEFYVTDTPKGPKATEGALNTP
KYLQPRTELL

TABLE 1H
>11_frag (500 aa)
YALVYFLQSINFVRIIMRLWLCWKCRSKNPLLYDANYFLC
WHGDGKMKDLSPRWYFYYLGTGPEAGLPYGANKDGIIWVA
TEGALNTPKDHIGTRNPEKYCALAPNMMVTNNTFTLKGGA
PTKVTFGTYKNTCDGTTFTYASALWEIQQVVDADLNESLI
DLQELGKYEQYIKWPWYIWLGFIAGLIAIVMVQTACTDDN
ALAYYNTTKGGRFVLALLSDLQDLKWARFPKSDGTGTIYT
ELEPPCRFVTDTPKGPKVKYLRVEAFEYYHTTDPSFLGRY
MSALNHTKKWITVATSRTLSYYKLGASQRVAGDSGFAAYS
RYRIGNYKLNTDHSSSSDNIALLVQVLQQLRVESSSKLWA
QCVQLHNDILLAKDTAPSASAFFGMSRIGMEVTPSGTWLT
YTGAIKLDDKDPNFKDQVILLNKHIDAYKTFPPTEPKKDK
SKLIEYTDFATSACVLAAECTIFKDASGKPVPYCYDTNVL
EGSVAYESLRPDTRYVLMDG

TABLE 1I
>s-pp-p2a-19_epitope_3aa (1588 aa)
MFVFLVLLPLVSSQCVNLTTRTQLPPAYTNSFTRGVYYPD
KVFRSSVLHSTQDLFLPFFSNVTWFHAIHVSGTNGTKRED
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTELLKYN
ENGTITDAVDCALDPLSETKCTLKSFTVEKGIYQTSNFRV
QPTESIVRFPNITNLCPFGEVENATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGENCYFPLQSYGFQPTNGVGYQPYRVVVLSFELLHA
PATVCGPKKSTNLVKNKCVNFNFNGLTGTGVLTESNKKEL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPTNFTI
SVTTEILPVSMTKTSVDCTMYICGDSTECSNLLLQYGSFC
TQLNRALTGIAVEQDKNTQEVFAQVKQIYKTPPIKDFGGF
NFSQILPDPSKPSKRSFIEDLLENKVTLADAGFIKQYGDC
LGDIAARDLICAQKENGLTVLPPLLTDEMIAQYTSALLAG
TITSGWTFGAGAALQIPFAMQMAYRFNGIGVTQNVLYENQ
KLIANQFNSAIGKIQDSLSSTASALGKLQDVVNQNAQALN
TLVKQLSSNFGAISSVLNDILSRLDPPEAEVQIDRLITGR
LQSLQTYVTQQLIRAAEIRASANLAATKMSECVLGQSKRV
DFCGKGYHLMSFPQSAPHGVVFLHVTYVPAQEKNFTTAPA
ICHDGKAHFPREGVFVSNGTHWFVTQRNFYEPQIITTDNT
FVSGNCDVVIGIVNNTVYDPLQPELDSFKEELDKYFKNHT
SPDVDLGDISGINASVVNIQKEIDRLNEVAKNLNESLIDL
QELGKYEQYIKWPWYIWLGFIAGLIAIVMVTIMLCCMTSC
CSCLKGCCSCGSCCKFDEDDSEPVLKGVKLHYTRAKRSGS
GATNFSLLKQAGDVEENPGPYASALWEIQQVVDADHSYFT
SDYYQLYSTQKHYVYIGDPAQLPAPDAYKTFPPTEPKKDK
IWVATEGALNTPKDHIITVATSRTLSYYKLGAYALVYFLQ
SINFVRISLEIPRRNVATLQAEPNMMVTNNTFTLKGGAKN
PLLYDANYFLCWHKDLSPRWYFYYLGTGYYHTTDPSFLGR
YMSAESLRPDTRYVLMDGRKVPTDNYITTYPGQCRFVTDT
PKGPKVKYSTFKCYGVSPTKLNDKYEQYIKWPWYIWLGSS
SKLWAQCVQLHNDYVGYLQPRTFLLKYN
>s-pp-p2a-27_epitope_3aa (1712 aa)*
MFVFLVLLPLVSSQCVNLTTRTQLPPAYTNSFTRGVYYPD
KVFRSSVLHSTQDLFLPFFSNVTWFHAIHVSGTNGTKRED
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTELLKYN
ENGTITDAVDCALDPLSETKCTLKSFTVEKGIYQTSNFRV
QPTESIVRFPNITNLCPFGEVENATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGENCYFPLQSYGFQPTNGVGYQPYRVVVLSFELLHA
PATVCGPKKSTNLVKNKCVNFNFNGLTGTGVLTESNKKEL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPINFTI
SVTTEILPVSMTKTSVDCTMYICGDSTECSNLLLQYGSFC
TQLNRALTGIAVEQDKNTQEVFAQVKQIYKTPPIKDFGGF
NFSQILPDPSKPSKRSFIEDLLFNKVTLADAGFIKQYGDC
LGDIAARDLICAQKENGLTVLPPLLTDEMIAQYTSALLAG
TITSGWTFGAGAALQIPFAMQMAYRFNGIGVTQNVLYENQ
KLIANQFNSAIGKIQDSLSSTASALGKLQDVVNQNAQALN
TLVKQLSSNFGAISSVLNDILSRLDPPEAEVQIDRLITGR
LQSLQTYVTQQLIRAAEIRASANLAATKMSECVLGQSKRV
DFCGKGYHLMSFPQSAPHGVVFLHVTYVPAQEKNFTTAPA
ICHDGKAHFPREGVFVSNGTHWFVTQRNFYEPQIITTDNT
FVSGNCDVVIGIVNNTVYDPLQPELDSFKEELDKYFKNHT
SPDVDLGDISGINASVVNIQKEIDRLNEVAKNLNESLIDL
QELGKYEQYIKWPWYIWLGFIAGLIAIVMVTIMLCCMTSC
CSCLKGCCSCGSCCKFDEDDSEPVLKGVKLHYTRAKRSGS
GATNFSLLKQAGDVEENPGPHSYFTSDYYQLYSTQKHYVY
IGDPAQLPAPYALVYFLQSINFVRIATYYLFDESGEFKLA
SHKNPLLYDANYFLCWHRKVPTDNYITTYPGQITVATSRT
LSYYKLGAPNMMVTNNTFTLKGGASIIKTIQPRVEKKKLK
YEQYIKWPWYIWLGKDLSPRWYFYYLGTGTYKNTCDGTTF
TYASAVHAGTDLEGNFYGPFESLRPDTRYVLMDGQTACTD
DNALAYYNTTSSSKLWAQCVQLHNDYASALWEIQQVVDAD
DAYKTFPPTEPKKDKSTFKCYGVSPTKLNDAPSASAFFGM
SRIGMLALLLLDRLNQLESKSLEIPRRNVATLQAEYYHTT
DPSFLGRYMSARDVDTDFVNEFYAYLCRFVTDTPKGPKVK
YIWVATEGALNTPKDHIYVGYLQPRTFLLKYN
*Note:
The 27 epitope fragment designated #2
in FIG.2 (cont.) is a more accurate
depiction of the construct labeled “27
epitope 3aa linker” on page 35.The
construct depiction in FIG.2 is an earlier
version of the construct depiction shown
FIG.2 (cont) and each of these construct
depictions is an earlier depiction
of the accurate construct depictions
provided in Figs.5-9.
>14_fragment (546 aa)
MLRVEAFEYYHTTDPSFLGRYMSALNHTKKWVLQQLRVES
SSKLWAQCVQLHNDILLAKDTAPSASAFFGMSRIGMEVTP
SGTWLTYTGAIKLDDKDPNFKDQVILLNKHIDAYKTFPPT
EPKKDKSLEIPRRNVATLQAELNESLIDLQELGKYEQYIK
WPWYIWLGFIAGLIAIVMVQTACTDDNALAYYNTTKGGRF
VLALLSDLQDLKWARFPKSDGTGTIYTELEPPCRFVTDTP
KGPKVKYSIIKTIQPRVEKKKLITVATSRTLSYYKLGASQ
RVAGDSGFAAYSRYRIGNYKLNTDHSSSSDNIALLVQSKL
IEYTDFATSACVLAAECTIFKDASGKPVPYCYDTNVLEGS
VAYESLRPDTRYVLMDGEKYCALAPNMMVTNNTFTLKGGA
PTKVTFGGDGKMKDLSPRWYFYYLGTGPEAGLPYGANKDG
IIWVATEGALNTPKDHIGTRNPYALVYFLQSINFVRIIMR
LWLCWKCRSKNPLLYDANYFLCWHTYKNTCDGTTFTYASA
LWEIQQVVDADKHYVYIGDPAQLPAP***
>B.1.617.2_S.PP_Epi-M/N/ORF3a (1099 aa)
MYASALWEIQQVVDADSSSKLWAQCVQLHNDKHYVYIGDP
AQLPAPSLEIPRRNVATLQAERDVDTDFVNEFYAYLTYKN
TCDGTTFTYASAPNMMVTNNTFTLKGGAATYYLFDESGEF
KLASHYYHTTDPSFLGRYMSAESLRPDTRYVLMDGQTACT
DDNALAYYNTTSIIKTIQPRVEKKKLCRFVTDTPKGPKVK
YSTFKCYGVSPTKLNDRKVPTDNYITTYPGQVHAGTDLEG
NFYGPFYVGYLQPRTFLLKYNGDGKMKDLSPRWYFYYLGT
GPEAGLPYGANKDGIIWVATEGALNTPKDHIGTRNPANNA
AIVLQLPQGTTLPKGFYAEGSRGGSQASSRSSSRSRNSSR
NSTPGEKWESGVKDCVVLHSYFTSDYYQLYSTQLSTDTGV
EHVTFFIYNKIVDEPEEHVQIHTIDGSSGVVNPVMEPIYD
EPTTTTSVPLSSMGTSPARMAGNGGDAALALLLLDRLNQL
ESKMSGKGQQQQGQTVTKKSAAEASKKPRQKRTATKAYNV
TQAFGRRGPEQTQGNFGDQELIRQGTDYKHWPQIAQFKQG
EIKDATPLDFVRATATIPIQASLPFGWLIVGVALLAVFQS
ASKIITLKKRWQLALSKGVHFVCNLLLLFVTVYSHLLLVA
AGLEAMSDNGPQNQRNAPRITFGGPSDSTGSNQNGERSGA
RSKQRRPQGLPNNTASWFTALTQHGKEGLKFPRGQGVPIN
TNSSPDDQIGYYRRATRRIRGLFARTRSMWSFNPETNILL
NVPLHGTILTRPLLESELVIGAVILRGHLRIAGHHLGRCD
IKDLPKEPFLYLYALVYFLQSINFVRIIMRLWLCWKCRSK
NPLLYDANYFLCWHTNCYDYCIPYNSVTSSIVITSGDGTT
SPISEHDYQIGGYTAPSASAFFGMSRIGMEVTPSGTWLTY
TGAIKLDDKDPNFKDQVILLNKHIDAYKTFPPTEPKKDKK
KKAYETQALPQRQKKQQTVTLLPAADLDDESKQLQQSMSS
ADSTQAITVATSRTLSYYKLGASQRVAGDSGFAAYSRYRI
GNYKLNTDHSSSSDNIALLVQLNESLIDLQELGKYEQYIK
WPWYIWLGFIAGLIAIVMV***
>B.1.617.2_S.PP_EpiFrag-M/N/ORF3a
(1221 aa)
MATYYLFDESGEFKLASHSKLIEYTDFATSACVLAAECTI
FKDASGKPVPYCYDTNVLEGSVAYESLRPDTRYVLMDGRK
VPTDNYITTYPGQQTACTDDNALAYYNTTKGGRFVLALLS
DLQDLKWARFPKSDGTGTIYTELEPPCRFVTDTPKGPKVK
YSTFKCYGVSPTKLNDRDVDTDFVNEFYAYLTYKNTCDGT
TFTYASALWEIQQVVDADLRVEAFEYYHTTDPSFLGRYMS
ALNHTKKWKHYVYIGDPAQLPAPSLEIPRRNVATLQAEVL
QQLRVESSSKLWAQCVQLHNDILLAKDTVHAGTDLEGNFY
GPFSIIKTIQPRVEKKKLEKYCALAPNMMVTNNTFTLKGG
APTKVTFGYVGYLQPRTFLLKYNGDGKMKDLSPRWYFYYL
GTGPEAGLPYGANKDGIIWVATEGALNTPKDHIGTRNPAN
NAAIVLQLPQGTTLPKGFYAEGSRGGSQASSRSSSRSRNS
SRNSTPGEKWESGVKDCVVLHSYFTSDYYQLYSTQLSTDT
GVEHVTFFIYNKIVDEPEEHVQIHTIDGSSGVVNPVMEPI
YDEPTTTTSVPLSSMGTSPARMAGNGGDAALALLLLDRLN
QLESKMSGKGQQQQGQTVTKKSAAEASKKPRQKRTATKAY
NVTQAFGRRGPEQTQGNFGDQELIRQGTDYKHWPQIAQFK
QGEIKDATPLDFVRATATIPIQASLPFGWLIVGVALLAVF
QSASKIITLKKRWQLALSKGVHFVCNLLLLFVTVYSHLLL
VAAGLEAMSDNGPQNQRNAPRITFGGPSDSTGSNQNGERS
GARSKQRRPQGLPNNTASWFTALTQHGKEGLKFPRGQGVP
INTNSSPDDQIGYYRRATRRIRGLFARTRSMWSFNPETNI
LLNVPLHGTILTRPLLESELVIGAVILRGHLRIAGHHLGR
CDIKDLPKEPFLYLYALVYFLQSINFVRIIMRLWLCWKCR
SKNPLLYDANYFLCWHTNCYDYCIPYNSVTSSIVITSGDG
TTSPISEHDYQIGGYTAPSASAFFGMSRIGMEVTPSGTWL
TYTGAIKLDDKDPNFKDQVILLNKHIDAYKTFPPTEPKKD
KKKKAYETQALPQRQKKQQTVTLLPAADLDDESKQLQQSM
SSADSTQAITVATSRTLSYYKLGASQRVAGDSGFAAYSRY
RIGNYKLNTDHSSSSDNIALLVQLNESLIDLQELGKYEQY
IKWPWYIWLGFIAGLIAIVMV***

TABLE 1J
>s-pp-p2a-11_frag (1800 aa)
MFVFLVLLPLVSSQCVNLTTRTQLPPAYTNSFTRGVYYPD
KVFRSSVLHSTQDLFLPFFSNVTWFHAIHVSGTNGTKRED
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVENATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGFNCYFPLQSYGFQPTNGVGYQPYRVVVLSFELLHA
PATVCGPKKSTNLVKNKCVNFNFNGLTGTGVLTESNKKEL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPTNFTI
SVTTEILPVSMTKTSVDCTMYICGDSTECSNLLLQYGSFC
TQLNRALTGIAVEQDKNTQEVFAQVKQIYKTPPIKDFGGF
NFSQILPDPSKPSKRSFIEDLLENKVTLADAGFIKQYGDC
LGDIAARDLICAQKENGLTVLPPLLTDEMIAQYTSALLAG
TITSGWTFGAGAALQIPFAMQMAYRENGIGVTQNVLYENQ
KLIANQFNSAIGKIQDSLSSTASALGKLQDVVNQNAQALN
TLVKQLSSNFGAISSVLNDILSRLDPPEAEVQIDRLITGR
LQSLQTYVTQQLIRAAEIRASANLAATKMSECVLGQSKRV
DFCGKGYHLMSFPQSAPHGVVFLHVTYVPAQEKNFTTAPA
ICHDGKAHFPREGVFVSNGTHWFVTQRNFYEPQIITTDNT
FVSGNCDVVIGIVNNTVYDPLQPELDSFKEELDKYFKNHT
SPDVDLGDISGINASVVNIQKEIDRLNEVAKNLNESLIDL
QELGKYEQYIKWPWYIWLGFIAGLIAIVMVTIMLCCMTSC
CSCLKGCCSCGSCCKFDEDDSEPVLKGVKLHYTRAKRSGS
GATNFSLLKQAGDVEENPGPYALVYFLQSINFVRIIMRLW
LCWKCRSKNPLLYDANYFLCWHGDGKMKDLSPRWYFYYLG
TGPEAGLPYGANKDGIIWVATEGALNTPKDHIGTRNPEKY
CALAPNMMVTNNTFTLKGGAPTKVTFGTYKNTCDGTTFTY
ASALWEIQQVVDADLNESLIDLQELGKYEQYIKWPWYIWL
GFIAGLIAIVMVQTACTDDNALAYYNTTKGGRFVLALLSD
LQDLKWARFPKSDGTGTIYTELEPPCRFVTDTPKGPKVKY
LRVEAFEYYHTTDPSFLGRYMSALNHTKKWITVATSRTLS
YYKLGASQRVAGDSGFAAYSRYRIGNYKLNTDHSSSSDNI
ALLVQVLQQLRVESSSKLWAQCVQLHNDILLAKDTAPSAS
AFFGMSRIGMEVTPSGTWLTYTGAIKLDDKDPNFKDQVIL
LNKHIDAYKTFPPTEPKKDKSKLIEYTDFATSACVLAAEC
TIFKDASGKPVPYCYDTNVLEGSVAYESLRPDTRYVLMDG

TABLE 1K
>YP_009724389 (SARS-COV-2 ORF1a/b protein)
MESLVPGFNEKTHVQLSLPVLQVRDVLVRGFGDSVEEVLS
EARQHLKDGTCGLVEVEKGVLPQLEQPYVFIKRSDARTAP
HGHVMVELVAELEGIQYGRSGETLGVLVPHVGEIPVAYRK
VLLRKNGNKGAGGHSYGADLKSFDLGDELGTDPYEDFQEN
WNTKHSSGVTRELMRELNGGAYTRYVDNNFCGPDGYPLEC
IKDLLARAGKASCTLSEQLDFIDTKRGVYCCREHEHEIAW
YTERSEKSYELQTPFEIKLAKKFDTFNGECPNFVFPLNSI
IKTIQPRVEKKKLDGFMGRIRSVYPVASPNECNQMCLSTL
MKCDHCGETSWQTGDFVKATCEFCGTENLTKEGATTCGYL
PQNAVVKIYCPACHNSEVGPEHSLAEYHNESGLKTILRKG
GRTIAFGGCVFSYVGCHNKCAYWVPRASANIGCNHTGVVG
EGSEGLNDNLLEILQKEKVNINIVGDFKLNEEIAIILASF
SASTSAFVETVKGLDYKAFKQIVESCGNFKVTKGKAKKGA
WNIGEQKSILSPLYAFASEAARVVRSIFSRTLETAQNSVR
VLQKAAITILDGISQYSLRLIDAMMFTSDLATNNLVVMAY
ITGGVVQLTSQWLTNIFGTVYEKLKPVLDWLEEKFKEGVE
FLRDGWEIVKFISTCACEIVGGQIVTCAKEIKESVQTFFK
LVNKFLALCADSIIIGGAKLKALNLGETFVTHSKGLYRKC
VKSREETGLLMPLKAPKEIIFLEGETLPTEVLTEEVVLKT
GDLQPLEQPTSEAVEAPLVGTPVCINGLMLLEIKDTEKYC
ALAPNMMVTNNTFTLKGGAPTKVTFGDDTVIEVQGYKSVN
ITFELDERIDKVLNEKCSAYTVELGTEVNEFACVVADAVI
KTLQPVSELLTPLGIDLDEWSMATYYLFDESGEFKLASHM
YCSFYPPDEDEEEGDCEEEEFEPSTQYEYGTEDDYQGKPL
EFGATSAALQPEEEQEEDWLDDDSQQTVGQQDGSEDNQTT
TIQTIVEVQPQLEMELTPVVQTIEVNSFSGYLKLTDNVYI
KNADIVEEAKKVKPTVVVNAANVYLKHGGGVAGALNKATN
NAMQVESDDYIATNGPLKVGGSCVLSGHNLAKHCLHVVGP
NVNKGEDIQLLKSAYENFNQHEVLLAPLLSAGIFGADPIH
SLRVCVDTVRTNVYLAVFDKNLYDKLVSSFLEMKSEKQVE
QKIAEIPKEEVKPFITESKPSVEQRKQDDKKIKACVEEVT
TTLEETKFLTENLLLYIDINGNLHPDSATLVSDIDITFLK
KDAPYIVGDVVQEGVLTAVVIPTKKAGGTTEMLAKALRKV
PTDNYITTYPGQGLNGYTVEEAKTVLKKCKSAFYILPSII
SNEKQEILGTVSWNLREMLAHAEETRKLMPVCVETKAIVS
TIQRKYKGIKIQEGVVDYGARFYFYTSKTTVASLINTLND
LNETLVTMPLGYVTHGLNLEEAARYMRSLKVPATVSVSSP
DAVTAYNGYLTSSSKTPEEHFIETISLAGSYKDWSYSGQS
TQLGIEFLKRGDKSVYYTSNPTTFHLDGEVITFDNLKTLL
SLREVRTIKVFTTVDNINLHTQVVDMSMTYGQQFGPTYLD
GADVTKIKPHNSHEGKTFYVLPNDDTLRVEAFEYYHTTDP
SFLGRYMSALNHTKKWKYPQVNGLTSIKWADNNCYLATAL
LTLQQIELKFNPPALQDAYYRARAGEAANFCALILAYCNK
TVGELGDVRETMSYLFQHANLDSCKRVLNVVCKTCGQQQT
TLKGVEAVMYMGTLSYEQFKKGVQIPCTCGKQATKYLVQQ
ESPFVMMSAPPAQYELKHGTFTCASEYTGNYQCGHYKHIT
SKETLYCIDGALLTKSSEYKGPITDVFYKENSYTTTIKPV
TYKLDGVVCTEIDPKLDNYYKKDNSYFTEQPIDLVPNQPY
PNASFDNFKFVCDNIKFADDLNQLTGYKKPASRELKVTFF
PDLNGDVVAIDYKHYTPSFKKGAKLLHKPIVWHVNNATNK
ATYKPNTWCIRCLWSTKPVETSNSFDVLKSEDAQGMDNLA
CEDLKPVSEEVVENPTIQKDVLECNVKTTEVVGDIILKPA
NNSLKITEEVGHTDLMAAYVDNSSLTIKKPNELSRVLGLK
TLATHGLAAVNSVPWDTIANYAKPFLNKVVSTTTNIVTRC
LNRVCTNYMPYFFTLLLQLCTFTRSTNSRIKASMPTTIAK
NTVKSVGKFCLEASFNYLKSPNFSKLINIIIWFLLLSVCL
GSLIYSTAALGVLMSNLGMPSYCTGYREGYLNSTNVTIAT
YCTGSIPCSVCLSGLDSLDTYPSLETIQITISSFKWDLTA
FGLVAEWFLAYILFTRFFYVLGLAAIMQLFFSYFAVHFIS
NSWLMWLIINLVQMAPISAMVRMYIFFASFYYVWKSYVHV
VDGCNSSTCMMCYKRNRATRVECTTIVNGVRRSFYVYANG
GKGFCKLHNWNCVNCDTFCAGSTFISDEVARDLSLQFKRP
INPTDQSSYIVDSVTVKNGSIHLYFDKAGQKTYERHSLSH
FVNLDNLRANNTKGSLPINVIVFDGKSKCEESSAKSASVY
YSQLMCQPILLLDQALVSDVGDSAEVAVKMFDAYVNTFSS
TFNVPMEKLKTLVATAEAELAKNVSLDNVLSTFISAARQG
FVDSDVETKDVVECLKLSHQSDIEVTGDSCNNYMLTYNKV
ENMTPRDLGACIDCSARHINAQVAKSHNIALIWNVKDFMS
LSEQLRKQIRSAAKKNNLPFKLTCATTRQVVNVVTTKIAL
KGGKIVNNWLKQLIKVTLVFLFVAAIFYLITPVHVMSKHT
DFSSEIIGYKAIDGGVTRDIASTDTCFANKHADFDTWFSQ
RGGSYTNDKACPLIAAVITREVGFVVPGLPGTILRTTNGD
FLHFLPRVFSAVGNICYTPSKLIEYTDFATSACVLAAECT
IFKDASGKPVPYCYDTNVLEGSVAYESLRPDTRYVLMDGS
IIQFPNTYLEGSVRVVTTFDSEYCRHGTCERSEAGVCVST
SGRWVLNNDYYRSLPGVFCGVDAVNLLTNMFTPLIQPIGA
LDISASIVAGGIVAIVVTCLAYYFMRFRRAFGEYSHVVAF
NTLLFLMSFTVLCLTPVYSFLPGVYSVIYLYLTFYLTNDV
SFLAHIQWMVMFTPLVPFWITIAYIICISTKHFYWFFSNY
LKRRVVFNGVSFSTFEEAALCTFLLNKEMYLKLRSDVLLP
LTQYNRYLALYNKYKYFSGAMDTTSYREAACCHLAKALND
FSNSGSDVLYQPPQTSITSAVLQSGFRKMAFPSGKVEGCM
VQVTCGTTTLNGLWLDDVVYCPRHVICTSEDMLNPNYEDL
LIRKSNHNFLVQAGNVQLRVIGHSMQNCVLKLKVDTANPK
TPKYKFVRIQPGQTFSVLACYNGSPSGVYQCAMRPNFTIK
GSFLNGSCGSVGFNIDYDCVSFCYMHHMELPTGVHAGTDL
EGNFYGPFVDRQTAQAAGTDTTITVNVLAWLYAAVINGDR
WFLNRFTTTLNDFNLVAMKYNYEPLTQDHVDILGPLSAQT
GIAVLDMCASLKELLQNGMNGRTILGSALLEDEFTPFDVV
RQCSGVTFQSAVKRTIKGTHHWLLLTILTSLLVLVQSTQW
SLFFFLYENAFLPFAMGIIAMSAFAMMFVKHKHAFLCLFL
LPSLATVAYFNMVYMPASWVMRIMTWLDMVDTSLSGFKLK
DCVMYASAVVLLILMTARTVYDDGARRVWTLMNVLTLVYK
VYYGNALDQAISMWALIISVTSNYSGVVTTVMFLARGIVF
MCVEYCPIFFITGNTLQCIMLVYCFLGYFCTCYFGLFCLL
NRYFRLTLGVYDYLVSTQEFRYMNSQGLLPPKNSIDAFKL
NIKLLGVGGKPCIKVATVQSKMSDVKCTSVVLLSVLQQLR
VESSSKLWAQCVQLHNDILLAKDTTEAFEKMVSLLSVLLS
MQGAVDINKLCEEMLDNRATLQAIASEFSSLPSYAAFATA
QEAYEQAVANGDSEVVLKKLKKSLNVAKSEFDRDAAMQRK
LEKMADQAMTQMYKQARSEDKRAKVTSAMQTMLFTMLRKL
DNDALNNIINNARDGCVPLNIIPLTTAAKLMVVIPDYNTY
KNTCDGTTFTYASALWEIQQVVDADSKIVQLSEISMDNSP
NLAWPLIVTALRANSAVKLQNNELSPVALRQMSCAAGTTQ
TACTDDNALAYYNTTKGGRFVLALLSDLQDLKWARFPKSD
GTGTIYTELEPPCRFVTDTPKGPKVKYLYFIKGLNNLNRG
MVLGSLAATVRLQAGNATEVPANSTVLSFCAFAVDAAKAY
KDYLASGGQPITNCVKMLCTHTGTGQAITVTPEANMDQES
FGGASCCLYCRCHIDHPNPKGFCDLKGKYVQIPTTCANDP
VGFTLKNTVCTVCGMWKGYGCSCDQLREPMLQSADAQSFL
NRVCGVSAARLTPCGTGTSTDVVYRAFDIYNDKVAGFAKF
LKTNCCRFQEKDEDDNLIDSYFVVKRHTFSNYQHEETIYN
LLKDCPAVAKHDFFKFRIDGDMVPHISRQRLTKYTMADLV
YALRHFDEGNCDTLKEILVTYNCCDDDYFNKKDWYDFVEN
PDILRVYANLGERVRQALLKTVQFCDAMRNAGIVGVLTLD
NQDLNGNWYDFGDFIQTTPGSGVPVVDSYYSLLMPILTLT
RALTAESHVDTDLTKPYIKWDLLKYDFTEERLKLFDRYFK
YWDQTYHPNCVNCLDDRCILHCANFNVLFSTVFPPTSFGP
LVRKIFVDGVPFVVSTGYHFRELGVVHNQDVNLHSSRLSF
KELLVYAADPAMHAASGNLLLDKRTTCFSVAALTNNVAFQ
TVKPGNFNKDFYDFAVSKGFFKEGSSVELKHFFFAQDGNA
AISDYDYYRYNLPTMCDIRQLLFVVEVVDKYFDCYDGGCI
NANQVIVNNLDKSAGFPFNKWGKARLYYDSMSYEDQDALF
AYTKRNVIPTITQMNLKYAISAKNRARTVAGVSICSTMTN
RQFHQKLLKSIAATRGATVVIGTSKFYGGWHNMLKTVYSD
VENPHLMGWDYPKCDRAMPNMLRIMASLVLARKHTTCCSL
SHRFYRLANECAQVLSEMVMCGGSLYVKPGGTSSGDATTA
YANSVFNICQAVTANVNALLSTDGNKIADKYVRNLQHRLY
ECLYRNRDVDTDFVNEFYAYLRKHFSMMILSDDAVVCFNS
TYASQGLVASIKNFKSVLYYQNNVFMSEAKCWTETDLTKG
PHEFCSQHTMLVKQGDDYVYLPYPDPSRILGAGCFVDDIV
KTDGTLMIERFVSLAIDAYPLTKHPNQEYADVFHLYLQYI
RKLHDELTGHMLDMYSVMLTNDNTSRYWEPEFYEAMYTPH
TVLQAVGACVLCNSQTSLRCGACIRRPFLCCKCCYDHVIS
TSHKLVLSVNPYVCNAPGCDVTDVTQLYLGGMSYYCKSHK
PPISFPLCANGQVFGLYKNTCVGSDNVTDFNAIATCDWTN
AGDYILANTCTERLKLFAAETLKATEETFKLSYGIATVRE
VLSDRELHLSWEVGKPRPPLNRNYVFTGYRVTKNSKVQIG
EYTFEKGDYGDAVVYRGTTTYKLNVGDYFVLTSHTVMPLS
APTLVPQEHYVRITGLYPTLNISDEFSSNVANYQKVGMQK
YSTLQGPPGTGKSHFAIGLALYYPSARIVYTACSHAAVDA
LCEKALKYLPIDKCSRIIPARARVECFDKFKVNSTLEQYV
FCTVNALPETTADIVVFDEISMATNYDLSVVNARLRAKHY
VYIGDPAQLPAPRTLLTKGTLEPEYFNSVCRLMKTIGPDM
FLGTCRRCPAEIVDTVSALVYDNKLKAHKDKSAQCFKMFY
KGVITHDVSSAINRPQIGVVREFLTRNPAWRKAVFISPYN
SQNAVASKILGLPTQTVDSSQGSEYDYVIFTQTTETAHSC
NVNRFNVAITRAKVGILCIMSDRDLYDKLQFTSLEIPRRN
VATLQAENVTGLFKDCSKVITGLHPTQAPTHLSVDTKFKT
EGLCVDIPGIPKDMTYRRLISMMGFKMNYQVNGYPNMFIT
REEAIRHVRAWIGFDVEGCHATREAVGTNLPLQLGFSTGV
NLVAVPTGYVDTPNNTDFSRVSAKPPPGDQFKHLIPLMYK
GLPWNVVRIKIVQMLSDTLKNLSDRVVFVLWAHGFELTSM
KYFVKIGPERTCCLCDRRATCFSTASDTYACWHHSIGFDY
VYNPFMIDVQQWGFTGNLQSNHDLYCQVHGNAHVASCDAI
MTRCLAVHECFVKRVDWTIEYPIIGDELKINAACRKVQHM
VVKAALLADKFPVLHDIGNPKAIKCVPQADVEWKFYDAQP
CSDKAYKIEELFYSYATHSDKFTDGVCLFWNCNVDRYPAN
SIVCRFDTRVLSNLNLPGCDGGSLYVNKHAFHTPAFDKSA
FVNLKQLPFFYYSDSPCESHGKQVVSDIDYVPLKSATCIT
RQNLGGAVCRHHANEYRLYLDAYNMMISAGFSLWVYKQFD
TYNLWNTFTRLQSLENVAFNVVNKGHFDGQQGEVPVSIIN
NTVYTKVDGVDVELFENKTTLPVNVAFELWAKRNIKPVPE
VKILNNLGVDIAANTVIWDYKRDAPAHISTIGVCSMTDIA
KKPTETICAPLTVFFDGRVDGQVDLFRNARNGVLITEGSV
KGLQPSVGPKQASLNGVTLIGEAVKTQFNYYKKVDGVVQQ
LPETYFTQSRNLQEFKPRSQMEIDFLELAMDEFIERYKLE
GYAFEHIVYGDFSHSQLGGLHLLIGLAKRFKESPFELEDF
IPMDSTVKNYFITDAQTGSSKCVCSVIDLLLDDFVEIIKS
QDLSVVSKVVKVTIDYTEISFMLWCKDGHVETFYPKLQSS
QAWQPGVAMPNLYKMQRMLLEKCDLQNYGDSATLPKGIMM
NVAKYTQLCQYLNTLTLAVPYNMRVIHFGAGSDKGVAPGT
AVLRQWLPTGTLLVDSDLNDFVSDADSTLIGDCATVHTAN
KWDLIISDMYDPKTKNVTKENDSKEGFFTYICGFIQQKLA
LGGSVAIKITEHSWNADLYKLMGHFAWWTAFVTNVNASSS
EAFLIGCNYLGKPREQIDGYVMHANYIFWRNTNPIQLSSY
SLFDMSKFPLKLRGTAVMSLKEGQINDMILSLLSKGRLII
RENNRVVISSDVLVNN
>YP_009724390 (SARS-COV-2 S protein)
MFVFLVLLPLVSSQCVNLTTRTQLPPAYTNSFTRGVYYPD
KVFRSSVLHSTQDLFLPFFSNVTWFHAIHVSGTNGTKRFD
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGFNCYFPLQSYGFQPTNGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPTNFTI
SVTTEILPVSMTKTSVDCTMYICGDSTECSNLLLQYGSFC
TQLNRALTGIAVEQDKNTQEVFAQVKQIYKTPPIKDFGGF
NFSQILPDPSKPSKRSFIEDLLFNKVTLADAGFIKQYGDC
LGDIAARDLICAQKFNGLTVLPPLLTDEMIAQYTSALLAG
TITSGWTFGAGAALQIPFAMQMAYRFNGIGVTQNVLYENQ
KLIANQFNSAIGKIQDSLSSTASALGKLQDVVNQNAQALN
TLVKQLSSNFGAISSVLNDILSRLDKVEAEVQIDRLITGR
LQSLQTYVTQQLIRAAEIRASANLAATKMSECVLGQSKRV
DFCGKGYHLMSFPQSAPHGVVFLHVTYVPAQEKNFTTAPA
ICHDGKAHFPREGVFVSNGTHWFVTQRNFYEPQIITTDNT
FVSGNCDVVIGIVNNTVYDPLQPELDSFKEELDKYFKNHT
SPDVDLGDISGINASVVNIQKEIDRLNEVAKNLNESLIDL
QELGKYEQYIKWPWYIWLGFIAGLIAIVMVTIMLCCMTSC
CSCLKGCCSCGSCCKFDEDDSEPVLKGVKLHYT
>YP_009724397 (SARS-COV-2 N protein)
MSDNGPQNQRNAPRITFGGPSDSTGSNQNGERSGARSKQR
RPQGLPNNTASWFTALTQHGKEDLKFPRGQGVPINTNSSP
DDQIGYYRRATRRIRGGDGKMKDLSPRWYFYYLGTGPEAG
LPYGANKDGIIWVATEGALNTPKDHIGTRNPANNAAIVLQ
LPQGTTLPKGFYAEGSRGGSQASSRSSSRSRNSSRNSTPG
SSRGTSPARMAGNGGDAALALLLLDRLNQLESKMSGKGQQ
QQGQTVTKKSAAEASKKPRQKRTATKAYNVTQAFGRRGPE
QTQGNFGDQELIRQGTDYKHWPQIAQFAPSASAFFGMSRI
GMEVTPSGTWLTYTGAIKLDDKDPNFKDQVILLNKHIDAY
KTFPPTEPKKDKKKKADETQALPQRQKKQQTVTLLPAADL
DDFSKQLQQSMSSADSTQA
>YP_009724391 (SARS-COV-2 orf3a protein)
MDLFMRIFTIGTVTLKQGEIKDATPSDFVRATATIPIQAS
LPFGWLIVGVALLAVFQSASKIITLKKRWQLALSKGVHFV
CNLLLLFVTVYSHLLLVAAGLEAPFLYLYALVYFLQSINF
VRIIMRLWLCWKCRSKNPLLYDANYFLCWHTNCYDYCIPY
NSVTSSIVITSGDGTTSPISEHDYQIGGYTEKWESGVKDC
VVLHSYFTSDYYQLYSTQLSTDTGVEHVTFFIYNKIVDEP
EEHVQIHTIDGSSGVVNPVMEPIYDEPTTTTSVPL
> YP_009724393.1 (SARS-COV-2 M protein)
MADSNGTITVEELKKLLEQWNLVIGFLFLTWICLLQFAYA
NRNRFLYIIKLIFLWLLWPVTLACFVLAAVYRINWITGGI
AIAMACLVGLMWLSYFIASFRLFARTRSMWSFNPETNILL
NVPLHGTILTRPLLESELVIGAVILRGHLRIAGHHLGRCD
IKDLPKEITVATSRTLSYYKLGASQRVAGDSGFAAYSRYR
IGNYKLNTDHSSSSDNIALLVQ
> YP_009724395.1 (SARS-COV-2 orf7a protein)
MKIILFLALITLATCELYHYQECVRGTTVLLKEPCSSGTY
EGNSPFHPLADNKFALTCFSTQFAFACPDGVKHVYQLRAR
SVSPKLFIRQEEVQELYSPIFLIVAAIVFITLCFTLKRKT
E

* Included in Tables 1A-1K are peptide epitopes, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity, or any range in between, inclusive, such as 85-99% identity, across their full length with an amino acid sequence of any SEQ ID NO listed in Tables 1A-1K, or a portion thereof. Such polypeptides may have a function of the full-length peptide or polypeptide as described further herein.

TABLE 2
>19_epitope_3aa_linker (870 nt)
Atgtacgccagcgccctgtgggagatccagcaggtggtggacgct
gatcacagctacttcacctctgattactaccagctttattctaca
cagaagcactacgtgtatatcggcgaccccgcccaactgcctgcc
cctgacgcctacaaaaccttcccccccacagaacccaaaaaggac
aaaatctgggtcgccaccgagggcgccctgaacacccctaaagac
catatcatcaccgtggccacaagcagaaccctgagctattacaag
ctgggcgcttacgccctggtgtactttctgcagagcatcaacttc
gtgcggatctccctggaaatccctaggcggaacgttgctaccctg
caggccgagcctaatatgatggtgaccaacaacaccttcaccctc
aagggcggagccaagaaccccctgctgtacgacgccaattacttc
ctgtgctggcacaaggacctgtctccaagatggtatttctactac
ctgggcaccggctactaccacaccacagatccaagcttcctggga
agatacatgagcgctgaaagcctgagacctgataccagatacgtg
ctgatggacggcagaaaggtgcctaccgacaactacattaccaca
taccccggccagtgtagattcgtgacagacacccctaagggacct
aaggtgaagtacagcacattcaagtgctacggcgtgtccccaaca
aagctgaatgataaatacgagcagtacatcaagtggccttggtac
atctggctgggcagcagctccaagctgtgggcccagtgcgtgcag
ctgcacaacgactacgtcggctacctgcaacctcggacatttctg
ctgaagtacaacTAG
>19_epitope_kaa_linker (690 nt)
Atggccctgtgggagatccagcaggttgtgaaggctgcattcacc
tctgattactaccagctgtacaaggccgccgtgtacatcggcgac
cccgcccagctgaaggccgctaaaaccttcccccccaccgagcct
aagaaagctgctgcaacagaaggcgccctgaatacacctaagaaa
gccgccgccacaagcagaacactgagctactacaagaaggccgct
gtgtatttcctccagagcatcaacttcaaggccgctatccccagg
cggaacgtggccaccctgaaagctgccatggtcaccaacaacacc
tttaccctgaagaaggccgccctgctgtacgacgccaattacttt
ctgaaggccgcatctccacggtggtacttctactacctgaaggcc
gccaccaccgaccctagcttcctgggcagatacaaggccgccaga
cctgacaccagatacgtgctgaaagccgcccctaccgacaactac
attacaacctacaaggccgccgtgacagatacccctaagggacct
aaaaaggccgccaagtgctacggcgtgtccccaacaaagaaggcc
gctcagtacatcaagtggccttggtatatcaaggccgccaagctg
tgggcccaatgtgtgcagctgaaggccgcctatctgcagcctaga
accttcctgctgTAG
>19_epitope_no_linker (528 nt)
Atggccctgtgggagatccagcaggtggtgttcaccagcgactac
taccagctgtacgtgtacatcggcgaccccgctcagctgaagacc
ttcccacctaccgaacccaaggccaccgagggagccctgaacacc
cctaaggctacatctagaaccctgagctactacaaggtgtacttc
ctgcagtctatcaacttcatcccccggagaaacgtggccaccctg
atggttaccaacaacacctttaccctgaagctgctgtacgacgcc
aattacttcctcagccctcggtggtatttctactaccttacaacc
gaccctagcttcctgggcagatacagacctgacacaagatatgtg
ctgcctacagataattacatcaccacatacgtcaccgataccccc
aaaggccctaaaaagtgctacggcgtgtcccctacaaagcagtac
attaagtggccttggtacatcaagctgtgggcccagtgtgtgcag
ctgtatctgcaaccacggacatttctgctgTAG
>27_epitope_3aa_linker (1248 nt)
atgcacagctacttcaccagcgactactaccagctgtacagcacc
cagaagcactacgtgtacatcggcgaccccgcccagctgcccgcc
ccctacgccctggtgtacttcctgcagagcatcaacttcgtgagg
atcgccacctactacctgttcgacgagagcggcgagttcaagctg
gccagccacaagaaccccctgctgtacgacgccaactacttcctg
tgctggcacaggaaggtgcccaccgacaactacatcaccacctac
cccggccagatcaccgtggccaccagcaggaccctgagctactac
aagctgggcgcccccaacatgatggtgaccaacaacaccttcacc
ctgaagggcggcgccagcatcatcaagaccatccagcccagggtg
gagaagaagaagctgaagtacgagcagtacatcaagtggccctgg
tacatctggctgggcaaggacctgagccccaggtggtacttctac
tacctgggcaccggcacctacaagaacacctgcgacggcaccacc
ttcacctacgccagcgccgtgcacgccggcaccgacctggagggc
aacttctacggccccttcgagagcctgaggcccgacaccaggtac
gtgctgatggacggccagaccgcctgcaccgacgacaacgccctg
gcctactacaacaccaccagcagcagcaagctgtgggcccagtgc
gtgcagctgcacaacgactacgccagcgccctgtgggagatccag
caggtggtggacgccgacgacgcctacaagaccttcccccccacc
gagcccaagaaggacaagagcaccttcaagtgctacggcgtgagc
cccaccaagctgaacgacgcccccagcgccagcgccttcttcggc
atgagcaggatcggcatgctggccctgctgctgctggacaggctg
aaccagctggagagcaagagcctggagatccccaggaggaacgtg
gccaccctgcaggccgagtactaccacaccaccgaccccagcttc
ctgggcaggtacatgagcgccagggacgtggacaccgacttcgtg
aacgagttctacgcctacctgtgcaggttcgtgaccgacaccccc
aagggccccaaggtgaagtacatctgggtggccaccgagggcgcc
ctgaacacccccaaggaccacatctacgtgggctacctgcagccc
aggaccttcctgctgaagtacaacTGATAATAG
>27_epitope_kaa_linker (990 nt)
Atgtttacgtcagactactatcagctttacaaagcagccgtctat
atcggggatcccgcccagcttaaagccgccgtgtatttcctgcag
tccatcaacttcaaagcagcgtatttgttcgatgagtctggggag
tttaaattgaaagctgccctcctctacgacgccaattattttctt
aaagcggccccaacggataactatataacgacctacaaagcagct
gccacatcccgaacactcagttattataagaaagcagccatggtt
acgaataacacttttacgctgaaaaaagctgcaaaaacgattcag
ccgcgggttgaaaagaaagctgcgcagtacataaagtggccatgg
tatatcaaagccgcctcccccagatggtacttctattatttgaaa
gcggccaacacatgcgacggtaccacgtttacatataaagctgcg
gggacagacttggaagggaatttttacaaggcggctcgcccagat
acacgctacgttttgaaagccgcttgtactgatgataacgcattg
gcttactataaagcagcaaagctttgggcccagtgcgttcagctg
aaggccgcagcgctctgggaaatccaacaggttgtgaaagccgca
aaaactttcccgccgacagaaccgaaaaaggcggcgaagtgctat
ggagtcagtcctactaaaaaggccgccgcgtcagccttctttggc
atgagtcgcaaggctgctctgcttttggatcggctcaatcaactc
aaagccgcaataccgagaaggaacgttgcgacattgaaggcggcc
acaacagacccgtcattcctgggcagatacaaggcagctgatacc
gacttcgtcaatgagttttacaaggcggcggtcactgacacaccg
aaaggccccaaaaaggccgctgcgaccgagggtgcgttgaataca
ccaaaaaaggcagcatatctccagccaaggacgttcctgctgTAG
>27_epitope_nolinker (756 nt)
atgttcacctctgattactaccagctttatgtgtacattggagat
cctgctcagctggtgtacttcctgcagagcatcaacttctacctg
ttcgacgagagcggcgaattcaagctgctgctgtacgacgccaac
tacttcctgcctaccgataattacatcaccacatacgccacatct
cggaccctgagctactacaagatggtcaccaacaacacctttacc
ctgaagaagaccatccagcctcgggtggaaaagcagtacatcaag
tggccttggtatatcagccctagatggtacttttactacctgaac
acctgcgacggcaccacctttacatacggcacagatctggaaggc
aacttctacagacctgacaccagatacgtgctgtgcaccgacgac
aatgccctggcctactataagctgtgggcccaatgtgtgcagctg
gctctgtgggagatccagcaggtggtgaagacattcccccccacc
gagcccaagaaatgctacggcgtgtccccaacaaaggccagcgcc
ttcttcggcatgagccggctgctgctggacagactgaaccagctg
atcccccgcaggaacgtggccaccctgaccaccgacccctccttc
ctgggaagatacgacacagacttcgtgaacgagttctacgttaca
gatacaccaaaaggccctaaagctacagagggcgccctgaatacc
cctaagtacctgcaacctagaacctttctgctcTAG
>11_frag (1506 nt)
atgtacgctctggtttattttcttcagtccatcaacttcgtaagg
atcattatgcgactctggctgtgctggaagtgtcgcagcaagaac
cccctcttgtatgacgctaattacttcctgtgctggcacggggac
ggcaagatgaaagatctgagccccagatggtacttctactatctt
ggtaccggacccgaagccgggctgccatacggggcaaataaagac
ggtattatctgggttgccactgagggtgccctgaatactcctaaa
gaccacattggcacacggaatcccgagaagtactgcgcattggct
cccaacatgatggtgaccaacaatacctttacattaaagggggga
gcaccaaccaaagtgacttttggcacttacaagaacacatgtgac
ggcacgacttttacctacgcaagcgctctgtgggaaattcaacag
gtcgtcgacgccgatcttaacgagtctcttatcgatttacaggaa
ttgggtaaatatgaacagtatatcaagtggccttggtacatctgg
ctaggctttatagcaggacttatagctatcgtcatggttcagacc
gcttgtacagacgataatgccctcgcctattataataccaccaaa
ggtgggcgttttgtgctggccctgctgtccgatctgcaggatctg
aagtgggctcgcttcccaaaaagtgacggaaccggcacgatctac
accgagttggagccaccttgtcgcttcgtgaccgatacgccaaaa
ggccctaaagtgaagtatctccgggttgaagcattcgaatactat
cataccacagatccttccttcctcgggaggtacatgagtgcccta
aatcatactaagaaatggattaccgtggccacttcacgaactctc
tcctactacaaactgggcgcttcacagcgggtagccggcgacagc
ggattcgccgcctactccaggtatagaattggaaactataagctg
aacacagaccattcttctagcagcgacaacatcgctctgttggtg
caggtcttgcaacagctgcgggtggagtcttcaagtaagttatgg
gcacaatgcgttcaacttcacaatgatattctactggcgaaagac
acggccccctctgcgtccgccttttttggaatgtctagaataggg
atggaagtaacaccatccggcacatggctcacatatacaggcgcg
atcaagttagacgacaaggacccgaatttcaaagatcaggtgatt
ctgctcaacaagcacatcgatgcatataagacatttccacctacg
gagcccaagaaggacaaaagcaagctcatagagtacactgatttc
gcaacctcggcttgtgtcctcgctgccgagtgcaccattttcaaa
gatgcctcagggaagcctgtgccgtattgctacgatacaaacgtg
cttgaaggttcggtggcgtacgagagtctgaggcccgatactaga
tatgtcctgatggacggaTAG
>s-pp-p2a-19_epitope_3aa (4767 nt)
atgttcgtgttcctggtgctgctccccctggtctctagccagtgc
gtgaatttaaccacacgcacccaactgccccccgcatacaccaac
tcctttaccagaggcgtgtactaccctgataaggtgtttagatct
agcgttttgcacagcacacaggacctcttcctgccattcttcagc
aatgtgacctggttccacgccattcacgtgtccggcacaaacggc
actaagagattcgacaaccccgtgctgccttttaatgacggcgtg
tactttgcatccaccgagaagtctaacatcatcagaggctggata
ttcggcaccaccctcgatagcaagacacagagcctgctcatcgtg
aacaacgccacaaacgtggtgatcaaggtgtgcgagtttcagttc
tgcaacgaccctttcctgggggtctactaccacaaaaacaacaag
agctggatggaatctgagttccgggtgtactccagcgccaataat
tgcaccttcgagtacgtgtcccagcccttcctgatggacttggag
ggcaagcagggcaattttaagaacctgagagagttcgtgtttaaa
aatatcgacggctacttcaagatctacagcaagcatacacctatc
aatctggtgagagacctgcctcagggcttcagcgcattggagccc
ctggttgacctgcccatcggcatcaacatcacaagattccagaca
ctgctggccctgcacagaagctacctgacccctggagattcctct
tctggatggaccgccggagccgccgcctactacgtgggatacctg
cagcctagaaccttcctactgaagtacaatgagaatggaaccatc
accgatgccgtggactgcgctctggaccccctgagcgagacaaag
tgtaccctgaagagcttcaccgtggaaaagggcatctatcagacc
agcaacttccgagtccagcctacagagagcattgtgcggttccct
aacatcacaaacctgtgcccttttggggaagtgttcaacgcgacc
cggttcgccagcgtgtatgcctggaacagaaaacggatctccaac
tgcgtggcagattacagcgtgctgtacaacagcgccagtttcagc
accttcaagtgctacggcgtgagccctacaaaactgaacgatctg
tgcttcaccaatgtgtacgccgactctttcgtgatcaggggcgac
gaagtgaggcagattgcccccggccagacgggcaaaatcgccgac
tacaactacaagctgcccgacgacttcaccggctgcgttattgcc
tggaactctaacaacctcgattctaaagtgggcggaaactacaac
tacctgtaccggctgttcagaaaaagcaacctgaaacctttcgag
agagacatatccacagaaatctaccaggctggctctactccttgt
aatggcgttgaagggttcaactgctactttccactgcagagctac
ggctttcagcctacaaacggagtgggctaccagccctaccgtgtg
gtggtgctgagcttcgaactgttgcacgcccccgctaccgtctgc
ggcccgaagaagagcacgaacctggtaaagaacaagtgcgtgaat
ttcaacttcaacggcctgacaggcaccggcgtgctgaccgaatcg
aacaagaagtttctgccatttcagcagttcggccgggacatcgcc
gacaccaccgatgccgtgcgggatcctcagacactagaaatcctg
gacatcaccccctgtagcttcggcggcgtgagcgtcatcacacct
ggcacaaacaccagcaaccaggtggccgtgctctaccaggacgtg
aattgcacagaggtgcctgtggccatccacgccgatcagctgacc
cctacatggcgggtctactctacaggaagcaacgtgttccagaca
agagccggatgccttataggcgccgagcacgtgaacaacagctac
gaatgcgacatccctatcggcgccggcatctgtgcttcttaccag
acacagactaactctccacggagagccagatcagtggcctctcag
tctatcatcgcctataccatgagtctgggagccgagaacagcgtg
gcatacagcaacaacagcatcgccatcccaaccaacttcacaatc
agtgtgacaaccgagatcctgcccgtgtccatgaccaagacctcg
gtggattgcacgatgtacatctgcggcgacagcaccgaatgcagc
aacctcctgctccagtacggcagtttctgcacacagctgaaccgg
gccctaaccggcatcgccgtggaacaggacaaaaacacccaggag
gtgttcgcccaagtgaagcaaatctacaagaccccacctatcaag
gacttcggcggcttcaattttagccagatcctgccagacccttct
aagccttcaaagagaagcttcatcgaggacctgttattcaacaaa
gtgacgctggccgatgctggcttcatcaagcaatacggcgactgc
ctcggcgacatcgcggcacgagacctgatctgcgcccagaagttt
aacggactgaccgtgttaccacctcttctgaccgatgaaatgatc
gcacagtacaccagcgccctcctggccggcaccatcaccagcgga
tggaccttcggagcgggcgctgcactgcagatccctttcgccatg
cagatggcctacagatttaatgggattggcgtgacccaaaacgtg
ctgtacgaaaaccagaaattgatcgctaaccaattcaactctgcc
attggaaagatccaggatagcctgagctctaccgcatccgctctg
ggcaagttgcaagacgtggtgaaccagaacgcccaggccctgaac
acgctggtgaaacagctgagcagcaacttcggcgctattagcagc
gtgctgaatgatattctgtcccggctggacccgcctgaggctgag
gtgcagattgatcggctgattacgggtcggctgcagagcctgcag
acctacgttacccagcagttgatcagagccgccgagatcagagcc
agtgcgaacctggcggcaaccaagatgagcgagtgtgtcctcgga
cagagcaagcgggttgacttctgtggaaagggctaccacctgatg
agcttcccccagtctgccccgcacggcgtggtgttcctgcacgtg
acctatgtgccagcccaggagaaaaatttcaccactgcccctgct
atctgtcatgatggcaaggcccacttccccagagaaggcgttttc
gtgagcaatggcacacactggttcgtgacacagcggaacttttac
gagcctcagatcatcacaactgacaatactttcgtgagtggcaac
tgcgacgtcgtcatcggcatcgtgaacaacaccgtgtacgaccct
ctgcaacctgagctggacagcttcaaggaagagctggataagtac
ttcaagaaccacaccagccccgacgtagatctgggcgacatcagc
ggcatcaacgccagcgtggtcaacatccagaaggagatcgacaga
ctgaacgaggttgccaagaatctgaacgaaagcctgatcgacctg
caggaactgggcaagtacgagcagtacatcaaatggccttggtac
atctggctgggcttcatcgccggcctgatcgccatcgtgatggtc
accatcatgctgtgttgcatgacctcatgctgctcttgcctgaag
ggctgttgtagctgcggctcttgttgcaagttcgacgaagatgac
agcgagcctgtgctcaagggcgtgaagctgcactacacccgggcc
aagcggagcggctccggcgccaccaattttagcctgctaaagcag
gccggcgacgtcgaggagaaccccggcccttatgccagcgctctg
tgggagatccagcaggtggtggacgccgaccacagctacttcacc
agcgactactaccagctgtactctacccaaaagcactacgtgtac
atcggcgatccagcccagctgccagcccctgatgcttacaagact
ttccccccaacagaacctaagaaagataaaatttgggtggccact
gaaggcgccctgaacacgcctaaggaccacatcatcacagtggcc
accagcagaacactgagctactacaagttaggcgcctacgctttg
gtgtatttcctgcagagcatcaacttcgtgcgcatcagcctggaa
atccccagaaggaacgtggccactctgcaggctgagcctaacatg
atggtgaccaacaacacctttaccctgaagggcggagccaagaac
cccctgctgtacgatgctaattacttcctgtgctggcacaaggat
ctgagtcctagatggtacttttactacctgggcacgggttactac
cacaccaccgaccccagcttcctgggcagatacatgtccgcagaa
agcctgagacctgataccagatacgtgctgatggacggcagaaag
gtgcctacagacaactacatcaccacctaccctggccagtgcaga
ttcgtaacggacacacctaagggccccaaggtgaagtacagcact
tttaagtgctacggagtgtctcctacaaagctgaacgataagtac
gagcagtacataaagtggccctggtacatttggctgggcagcagt
agcaagctatgggcccagtgtgttcagctgcataacgactacgtg
ggatacctgcagccaagaacctttctgctgaagtacaacTAG
>s-pp-p2a-27_epitope_3aa (5139 nt)
atgttcgtgttcctggtgctgctccccctggtctctagccagtgc
gtgaatttaaccacacgcacccaactgccccccgcatacaccaac
tcctttaccagaggcgtgtactaccctgataaggtgtttagatct
agcgttttgcacagcacacaggacctcttcctgccattcttcagc
aatgtgacctggttccacgccattcacgtgtccggcacaaacggc
actaagagattcgacaaccccgtgctgccttttaatgacggcgtg
tactttgcatccaccgagaagtctaacatcatcagaggctggata
ttcggcaccaccctcgatagcaagacacagagcctgctcatcgtg
aacaacgccacaaacgtggtgatcaaggtgtgcgagtttcagttc
tgcaacgaccctttcctgggggtctactaccacaaaaacaacaag
agctggatggaatctgagttccgggtgtactccagcgccaataat
tgcaccttcgagtacgtgtcccagcccttcctgatggacttggag
ggcaagcagggcaattttaagaacctgagagagttcgtgtttaaa
aatatcgacggctacttcaagatctacagcaagcatacacctatc
aatctggtgagagacctgcctcagggcttcagcgcattggagccc
ctggttgacctgcccatcggcatcaacatcacaagattccagaca
ctgctggccctgcacagaagctacctgacccctggagattcctct
tctggatggaccgccggagccgccgcctactacgtgggatacctg
cagcctagaaccttcctactgaagtacaatgagaatggaaccatc
accgatgccgtggactgcgctctggaccccctgagcgagacaaag
tgtaccctgaagagcttcaccgtggaaaagggcatctatcagacc
agcaacttccgagtccagcctacagagagcattgtgcggttccct
aacatcacaaacctgtgcccttttggggaagtgttcaacgcgacc
cggttcgccagcgtgtatgcctggaacagaaaacggatctccaac
tgcgtggcagattacagcgtgctgtacaacagcgccagtttcagc
accttcaagtgctacggcgtgagccctacaaaactgaacgatctg
tgcttcaccaatgtgtacgccgactctttcgtgatcaggggcgac
gaagtgaggcagattgcccccggccagacgggcaaaatcgccgac
tacaactacaagctgcccgacgacttcaccggctgcgttattgcc
tggaactctaacaacctcgattctaaagtgggcggaaactacaac
tacctgtaccggctgttcagaaaaagcaacctgaaacctttcgag
agagacatatccacagaaatctaccaggctggctctactccttgt
aatggcgttgaagggttcaactgctactttccactgcagagctac
ggctttcagcctacaaacggagtgggctaccagccctaccgtgtg
gtggtgctgagcttcgaactgttgcacgcccccgctaccgtctgc
ggcccgaagaagagcacgaacctggtaaagaacaagtgcgtgaat
ttcaacttcaacggcctgacaggcaccggcgtgctgaccgaatcg
aacaagaagtttctgccatttcagcagttcggccgggacatcgcc
gacaccaccgatgccgtgcgggatcctcagacactagaaatcctg
gacatcaccccctgtagcttcggcggcgtgagcgtcatcacacct
ggcacaaacaccagcaaccaggtggccgtgctctaccaggacgtg
aattgcacagaggtgcctgtggccatccacgccgatcagctgacc
cctacatggcgggtctactctacaggaagcaacgtgttccagaca
agagccggatgccttataggcgccgagcacgtgaacaacagctac
gaatgcgacatccctatcggcgccggcatctgtgcttcttaccag
acacagactaactctccacggagagccagatcagtggcctctcag
tctatcatcgcctataccatgagtctgggagccgagaacagcgtg
gcatacagcaacaacagcatcgccatcccaaccaacttcacaatc
agtgtgacaaccgagatcctgcccgtgtccatgaccaagacctcg
gtggattgcacgatgtacatctgcggcgacagcaccgaatgcagc
aacctcctgctccagtacggcagtttctgcacacagctgaaccgg
gccctaaccggcatcgccgtggaacaggacaaaaacacccaggag
gtgttcgcccaagtgaagcaaatctacaagaccccacctatcaag
gacttcggcggcttcaattttagccagatcctgccagacccttct
aagccttcaaagagaagcttcatcgaggacctgttattcaacaaa
gtgacgctggccgatgctggcttcatcaagcaatacggcgactgc
ctcggcgacatcgcggcacgagacctgatctgcgcccagaagttt
aacggactgaccgtgttaccacctcttctgaccgatgaaatgatc
gcacagtacaccagcgccctcctggccggcaccatcaccagcgga
tggaccttcggagcgggcgctgcactgcagatccctttcgccatg
cagatggcctacagatttaatgggattggcgtgacccaaaacgtg
ctgtacgaaaaccagaaattgatcgctaaccaattcaactctgcc
attggaaagatccaggatagcctgagctctaccgcatccgctctg
ggcaagttgcaagacgtggtgaaccagaacgcccaggccctgaac
acgctggtgaaacagctgagcagcaacttcggcgctattagcagc
gtgctgaatgatattctgtcccggctggacccgcctgaggctgag
gtgcagattgatcggctgattacgggtcggctgcagagcctgcag
acctacgttacccagcagttgatcagagccgccgagatcagagcc
agtgcgaacctggcggcaaccaagatgagcgagtgtgtcctcgga
cagagcaagcgggttgacttctgtggaaagggctaccacctgatg
agcttcccccagtctgccccgcacggcgtggtgttcctgcacgtg
acctatgtgccagcccaggagaaaaatttcaccactgcccctgct
atctgtcatgatggcaaggcccacttccccagagaaggcgttttc
gtgagcaatggcacacactggttcgtgacacagcggaacttttac
gagcctcagatcatcacaactgacaatactttcgtgagtggcaac
tgcgacgtcgtcatcggcatcgtgaacaacaccgtgtacgaccct
ctgcaacctgagctggacagcttcaaggaagagctggataagtac
ttcaagaaccacaccagccccgacgtagatctgggcgacatcagc
ggcatcaacgccagcgtggtcaacatccagaaggagatcgacaga
ctgaacgaggttgccaagaatctgaacgaaagcctgatcgacctg
caggaactgggcaagtacgagcagtacatcaaatggccttggtac
atctggctgggcttcatcgccggcctgatcgccatcgtgatggtc
accatcatgctgtgttgcatgacctcatgctgctcttgcctgaag
ggctgttgtagctgcggctcttgttgcaagttcgacgaagatgac
agcgagcctgtgctcaagggcgtgaagctgcactacacccgggcc
aagcggagcggctccggcgccaccaattttagcctgctaaagcag
gccggcgacgtcgaggagaaccccggccctcactcctactttaca
agtgattactaccaactgtactcaacacagaagcactacgtgtac
atcggcgatcctgcccagctgccagccccttacgccctagtgtat
tttctgcaatctatcaacttcgtccggattgccacgtactatctg
ttcgacgagtctggcgaattcaaactggcctcccacaagaaccct
ctgctgtatgacgctaactacttcctatgctggcacagaaaggtg
cccaccgataactacatcacaacctaccctggccagataaccgtg
gccacaagcagaacactgtcctactacaagctgggtgctcctaat
atgatggtgaccaacaacacattcaccctgaagggcggagccagt
atcatcaagaccatccagcctagagtggaaaagaaaaagctgaag
tacgagcagtacatcaagtggccttggtatatctggctgggaaaa
gacctgagccccagatggtacttttactatctgggcacaggcacc
tacaagaacacctgtgacggcacaacattcacctacgcctctgcc
gtgcacgccggaacagacctggaaggcaacttctacggacctttc
gagagcctgagacccgataccaggtacgtgctgatggatggccag
accgcctgtacagacgacaatgctctggcctactataacaccact
agcagctcaaagctgtgggcccagtgcgtgcagctgcataacgat
tacgccagcgccctgtgggagatccaacaggtggtggacgccgac
gacgcttataagaccttccctcccaccgagcccaagaaggacaag
agtaccttcaaatgctacggcgtttctcccaccaaactgaacgac
gctcctagcgccagtgccttctttggaatgtcaagaatcggcatg
cttgccctgctgctgctcgatagactgaaccagctggaatccaag
agccttgagatccccagaagaaacgtggctaccctgcaggccgag
tactaccacaccaccgacccttccttcctggggagatacatgagc
gccagagacgtcgatactgacttcgtaaatgagttctacgcctac
ctgtgtagattcgtcacagacaccccaaagggccctaaggtcaag
tacatttgggtggccaccgaaggcgctctgaacactcctaaggat
cacatctatgtcggctacctgcagcctcggacattcctgctgaaa
tacaacTAG
>14_fragment (1647 nt)
atgctgagggtggaggccttcgagtactaccacaccaccgacccc
agcttcctgggcaggtacatgagcgccctgaaccacaccaagaag
tgggtgctgcagcagctgagggtggagagcagcagcaagctgtgg
gcccagtgcgtgcagctgcacaacgacatcctgctggccaaggac
accgcccccagcgccagcgccttcttcggcatgagcaggatcggc
atggaggtgacccccagcggcacctggctgacctacaccggcgcc
atcaagctggacgacaaggaccccaacttcaaggaccaggtgatc
ctgctgaacaagcacatcgacgcctacaagaccttcccccccacc
gagcccaagaaggacaagagcctggagatccccaggaggaacgtg
gccaccctgcaggccgagctgaacgagagcctgatcgacctgcag
gagctgggcaagtacgagcagtacatcaagtggccctggtacatc
tggctgggcttcatcgccggcctgatcgccatcgtgatggtgcag
accgcctgcaccgacgacaacgccctggcctactacaacaccacc
aagggcggcaggttcgtgctggccctgctgagcgacctgcaggac
ctgaagtgggccaggttccccaagagcgacggcaccggcaccatc
tacaccgagctggagcccccctgcaggttcgtgaccgacaccccc
aagggccccaaggtgaagtacagcatcatcaagaccatccagccc
agggtggagaagaagaagctgatcaccgtggccaccagcaggacc
ctgagctactacaagctgggcgccagccagagggtggccggcgac
agcggcttcgccgcctacagcaggtacaggatcggcaactacaag
ctgaacaccgaccacagcagcagcagcgacaacatcgccctgctg
gtgcagagcaagctgatcgagtacaccgacttcgccaccagcgcc
tgcgtgctggccgccgagtgcaccatcttcaaggacgccagcggc
aagcccgtgccctactgctacgacaccaacgtgctggagggcagc
gtggcctacgagagcctgaggcccgacaccaggtacgtgctgatg
gacggcgagaagtactgcgccctggcccccaacatgatggtgacc
aacaacaccttcaccctgaagggcggcgcccccaccaaggtgacc
ttcggcggcgacggcaagatgaaggacctgagccccaggtggtac
ttctactacctgggcaccggccccgaggccggcctgccctacggc
gccaacaaggacggcatcatctgggtggccaccgagggcgccctg
aacacccccaaggaccacatcggcaccaggaacccctacgccctg
gtgtacttcctgcagagcatcaacttcgtgaggatcatcatgagg
ctgtggctgtgctggaagtgcaggagcaagaaccccctgctgtac
gacgccaactacttcctgtgctggcacacctacaagaacacctgc
gacggcaccaccttcacctacgccagcgccctgtgggagatccag
caggtggtggacgccgacaagcactacgtgtacatcggcgacccc
gcccagctgcccgcccccTGATAATAG
>B.1.617.2_S.PP_Epi-M/N/ORF3a (3306 nt)
atgtacgccagcgccctgtgggagatccagcaggtggtggacgcc
gacagcagcagcaagctgtgggcccagtgcgtgcagctgcacaac
gacaagcactacgtgtacatcggcgaccccgcccagctgcccgcc
cccagcctggagatccccagaagaaacgtggccaccctgcaggcc
gagagagacgtggacaccgacttcgtgaacgagttctacgcctac
ctgacctacaagaacacctgcgacggcaccaccttcacctacgcc
agcgcccccaacatgatggtgaccaacaacaccttcaccctgaag
ggcggcgccgccacctactacctgttcgacgagagcggcgagttc
aagctggccagccactactaccacaccaccgaccccagcttcctg
ggcagatacatgagcgccgagagcctgagacccgacaccagatac
gtgctgatggacggccagaccgcctgcaccgacgacaacgccctg
gcctactacaacaccaccagcatcatcaagaccatccagcccaga
gtggagaagaagaagctgtgcagattcgtgaccgacacccccaag
ggccccaaggtgaagtacagcaccttcaagtgctacggcgtgagc
cccaccaagctgaacgacagaaaggtgcccaccgacaactacatc
accacctaccccggccaggtgcacgccggcaccgacctggagggc
aacttctacggccccttctacgtgggctacctgcagcccagaacc
ttcctgctgaagtacaacggcgacggcaagatgaaggacctgagc
cccagatggtacttctactacctgggcaccggccccgaggccggc
ctgccctacggcgccaacaaggacggcatcatctgggtggccacc
gagggcgccctgaacacccccaaggaccacatcggcaccagaaac
cccgccaacaacgccgccatcgtgctgcagctgccccagggcacc
accctgcccaagggcttctacgccgagggcagcagaggcggcagc
caggccagcagcagaagcagcagcagaagcagaaacagcagcaga
aacagcacccccggcgagaagtgggagagcggcgtgaaggactgc
gtggtgctgcacagctacttcaccagcgactactaccagctgtac
agcacccagctgagcaccgacaccggcgtggagcacgtgaccttc
ttcatctacaacaagatcgtggacgagcccgaggagcacgtgcag
atccacaccatcgacggcagcagcggcgtggtgaaccccgtgatg
gagcccatctacgacgagcccaccaccaccaccagcgtgcccctg
agcagcatgggcaccagccccgccagaatggccggcaacggcggc
gacgccgccctggccctgctgctgctggacagactgaaccagctg
gagagcaagatgagcggcaagggccagcagcagcagggccagacc
gtgaccaagaagagcgccgccgaggccagcaagaagcccagacag
aagagaaccgccaccaaggcctacaacgtgacccaggccttcggc
agaagaggccccgagcagacccagggcaacttcggcgaccaggag
ctgatcagacagggcaccgactacaagcactggccccagatcgcc
cagttcaagcagggcgagatcaaggacgccacccccctggacttc
gtgagagccaccgccaccatccccatccaggccagcctgcccttc
ggctggctgatcgtgggcgtggccctgctggccgtgttccagagc
gccagcaagatcatcaccctgaagaagagatggcagctggccctg
agcaagggcgtgcacttcgtgtgcaacctgctgctgctgttcgtg
accgtgtacagccacctgctgctggtggccgccggcctggaggcc
atgagcgacaacggcccccagaaccagagaaacgcccccagaatc
accttcggcggccccagcgacagcaccggcagcaaccagaacggc
gagagaagggcgccagaagcaagcagagaagaccccagggcctgc
ccaacaacaccgccagctggttcaccgccctgacccagcacggca
aggagggcctgaagttccccagaggccagggcgtgcccatcaaca
ccaacagcagccccgacgaccagatcggctactacagaagagcca
ccagaagaatcagaggcctgttcgccagaaccagaagcatgtgga
gcttcaaccccgagaccaacatcctgctgaacgtgcccctgcacg
gcaccatcctgaccagacccctgctggagagcgagctggtgatcg
gcgccgtgatcctgagaggccacctgagaatcgccggccaccacc
tgggcagatgcgacatcaaggacctgcccaaggagcccttcctgt
acctgtacgccctggtgtacttcctgcagagcatcaacttcgtga
gaatcatcatgagactgtggctgtgctggaagtgcagaagcaaga
accccctgctgtacgacgccaactacttcctgtgctggcacacca
actgctacgactactgcatcccctacaacagcgtgaccagcagca
tcgtgatcaccagcggcgacggcaccaccagccccatcagcgagc
acgactaccagatcggcggctacaccgcccccagcgccagcgcct
tcttcggcatgagcagaatcggcatggaggtgacccccagcggca
cctggctgacctacaccggcgccatcaagctggacgacaaggacc
ccaacttcaaggaccaggtgatcctgctgaacaagcacatcgacg
cctacaagaccttcccccccaccgagcccaagaaggacaagaaga
agaaggcctacgagacccaggccctgccccagagacagaagaagc
agcagaccgtgaccctgctgcccgccgccgacctggacgacttca
gcaagcagctgcagcagagcatgagcagcgccgacagcacccagg
ccatcaccgtggccaccagcagaaccctgagctactacaagctgg
gcgccagccagagagtggccggcgacagcggcttcgccgcctaca
gcagatacagaatcggcaactacaagctgaacaccgaccacagca
gcagcagcgacaacatcgccctgctggtgcagctgaacgagagcc
tgatcgacctgcaggagctgggcaagtacgagcagtacatcaagt
ggccctggtacatctggctgggcttcatcgccggcctgatcgcca
tcgtgatggtgTGATAATAG
>B.1.617.2_S.PP_EpiFrag-M/N/ORF3a (3672 nt)
atggccacctactacctgttcgacgagagcggcgagttcaagctg
gccagccacagcaagctgatcgagtacaccgacttcgccaccagc
gcctgcgtgctggccgccgagtgcaccatcttcaaggacgccagc
ggcaagcccgtgccctactgctacgacaccaacgtgctggagggc
agcgtggcctacgagagcctgagacccgacaccagatacgtgctg
atggacggcagaaaggtgcccaccgacaactacatcaccacctac
cccggccagcagaccgcctgcaccgacgacaacgccctggcctac
tacaacaccaccaagggcggcagattcgtgctggccctgctgagc
gacctgcaggacctgaagtgggccagattccccaagagcgacggc
accggcaccatctacaccgagctggagcccccctgcagattcgtg
accgacacccccaagggccccaaggtgaagtacagcaccttcaag
tgctacggcgtgagccccaccaagctgaacgacagagacgtggac
accgacttcgtgaacgagttctacgcctacctgacctacaagaac
acctgcgacggcaccaccttcacctacgccagcgccctgtgggag
atccagcaggtggtggacgccgacctgagagtggaggccttcgag
tactaccacaccaccgaccccagcttcctgggcagatacatgagc
gccctgaaccacaccaagaagtggaagcactacgtgtacatcggc
gaccccgcccagctgcccgcccccagcctggagatccccagaaga
aacgtggccaccctgcaggccgaggtgctgcagcagctgagagtg
gagagcagcagcaagctgtgggcccagtgcgtgcagctgcacaac
gacatcctgctggccaaggacaccgtgcacgccggcaccgacctg
gagggcaacttctacggccccttcagcatcatcaagaccatccag
cccagagtggagaagaagaagctggagaagtactgcgccctggcc
cccaacatgatggtgaccaacaacaccttcaccctgaagggcggc
gcccccaccaaggtgaccttcggctacgtgggctacctgcagccc
agaaccttcctgctgaagtacaacggcgacggcaagatgaaggac
ctgagccccagatggtacttctactacctgggcaccggccccgag
gccggcctgccctacggcgccaacaaggacggcatcatctgggtg
gccaccgagggcgccctgaacacccccaaggaccacatcggcacc
agaaaccccgccaacaacgccgccatcgtgctgcagctgccccag
ggcaccaccctgcccaagggcttctacgccgagggcagcagaggc
ggcagccaggccagcagcagaagcagcagcagaagcagaaacagc
agcagaaacagcacccccggcgagaagtgggagagcggcgtgaag
gactgcgtggtgctgcacagctacttcaccagcgactactaccag
ctgtacagcacccagctgagcaccgacaccggcgtggagcacgtg
accttcttcatctacaacaagatcgtggacgagcccgaggagcac
gtgcagatccacaccatcgacggcagcagcggcgtggtgaacccc
gtgatggagcccatctacgacgagcccaccaccaccaccagcgtg
cccctgagcagcatgggcaccagccccgccagaatggccggcaac
ggcggcgacgccgccctggccctgctgctgctggacagactgaac
cagctggagagcaagatgagcggcaagggccagcagcagcagggc
cagaccgtgaccaagaagagcgccgccgaggccagcaagaagccc
agacagaagagaaccgccaccaaggcctacaacgtgacccaggcc
ttcggcagaagaggccccgagcagacccagggcaacttcggcgac
caggagctgatcagacagggcaccgactacaagcactggccccag
atcgcccagttcaagcagggcgagatcaaggacgccacccccctg
gacttcgtgagagccaccgccaccatccccatccaggccagcctg
cccttcggctggctgatcgtgggcgtggccctgctggccgtgttc
cagagcgccagcaagatcatcaccctgaagaagagatggcagctg
gccctgagcaagggcgtgcacttcgtgtgcaacctgctgctgctg
ttcgtgaccgtgtacagccacctgctgctggtggccgccggcctg
gaggccatgagcgacaacggcccccagaaccagagaaacgccccc
agaatcaccttcggcggccccagcgacagcaccggcagcaaccag
aacggcgagagaagcggcgccagaagcaagcagagaagaccccag
ggcctgcccaacaacaccgccagctggttcaccgccctgacccag
cacggcaaggagggcctgaagttccccagaggccagggcgtgccc
atcaacaccaacagcagccccgacgaccagatcggctactacaga
agagccaccagaagaatcagaggcctgttcgccagaaccagaagc
atgtggagcttcaaccccgagaccaacatcctgctgaacgtgccc
ctgcacggcaccatcctgaccagacccctgctggagagcgagctg
gtgatcggcgccgtgatcctgagaggccacctgagaatcgccggc
caccacctgggcagatgcgacatcaaggacctgcccaaggagccc
ttcctgtacctgtacgccctggtgtacttcctgcagagcatcaac
ttcgtgagaatcatcatgagactgtggctgtgctggaagtgcaga
agcaagaaccccctgctgtacgacgccaactacttcctgtgctgg
cacaccaactgctacgactactgcatcccctacaacagcgtgacc
agcagcatcgtgatcaccagcggcgacggcaccaccagccccatc
agcgagcacgactaccagatcggcggctacaccgcccccagcgcc
agcgccttcttcggcatgagcagaatcggcatggaggtgaccccc
agcggcacctggctgacctacaccggcgccatcaagctggacgac
aaggaccccaacttcaaggaccaggtgatcctgctgaacaagcac
atcgacgcctacaagaccttcccccccaccgagcccaagaaggac
aagaagaagaaggcctacgagacccaggccctgccccagagacag
aagaagcagcagaccgtgaccctgctgcccgccgccgacctggac
gacttcagcaagcagctgcagcagagcatgagcagcgccgacagc
acccaggccatcaccgtggccaccagcagaaccctgagctactac
aagctgggcgccagccagagagtggccggcgacagcggcttcgcc
gcctacagcagatacagaatcggcaactacaagctgaacaccgac
cacagcagcagcagcgacaacatcgccctgctggtgcagctgaac
gagagcctgatcgacctgcaggagctgggcaagtacgagcagtac
atcaagtggccctggtacatctggctgggcttcatcgccggcctg
atcgccatcgtgatggtgTGATAATA
G
>s-pp-p2a-11_frag (5403 nt)
atgttcgtttttctggttctgctgccactcgtgtcaagtcagtgc
gtgaaccttactacaaggactcagctcccaccagcatacacgaat
agttttacgcggggcgtgtactatccagacaaagtgtttcgcagc
tctgttctacattcaactcaagacctgtttttgcctttcttctcc
aatgtgacctggttccacgccatccacgtgagtggcacgaacggg
accaaacggtttgataacccagtgctgccttttaacgacggggta
tatttcgcctctactgaaaaatccaacatcatccgcggctggatt
ttcgggaccactcttgactccaagacccagtcactcctgatcgta
aacaatgcgaccaacgtcgtgattaaggtgtgcgagtttcaattc
tgtaacgaccctttcctgggtgtatattaccacaagaataataag
tcctggatggaatcagagtttagagtatactctagcgctaacaac
tgtacttttgaatatgtgtcccaacccttcttgatggacttggag
ggaaaacagggaaattttaagaatctccgagagttcgtgtttaaa
aacattgacggctatttcaagatatactctaagcatacacccatc
aatctggtccgcgatctgccacaggggtttagcgcactggaaccg
ttggtggatctccccattgggattaatatcacccgtttccagaca
cttttagccttgcatcggagctacctaacccccggggactcaagt
agcggctggactgcgggagcggccgcctattatgtcggatatctg
cagcctcggacattcctcctgaagtacaatgagaatggcacaatt
acagacgcagtagactgtgccctggatccgctctccgaaaccaaa
tgcacgctgaaatcatttacggtggaaaaaggtatataccagacc
agcaatttcagggtgcagcctacggagtccattgtccgtttcccc
aatatcaccaatctgtgtcctttcggcgaagtgtttaacgcaact
aggttcgcgagtgtctacgcctggaaccgaaagagaatctcaaac
tgtgtggccgattacagcgtcctgtacaactccgcatctttcagt
accttcaagtgctacggggtcagccccaccaaacttaacgatctt
tgcttcactaacgtttatgccgatagttttgtcatcaggggcgac
gaagtgcgacagattgcccctggacagacgggaaagatcgccgac
tataactataagctgccagacgatttcacaggatgcgtgatcgcc
tggaatagcaacaatctggactctaaggtgggggggaattataat
tatttgtatagactgtttcgaaagtcaaaccttaagccatttgag
agggatatcagcacagagatttaccaggcaggaagcaccccatgt
aacggggtagaaggcttcaactgctacttccccctccagtcatat
gggttccagcctaccaatggtgtgggttaccagccgtacagagta
gtggttctttcatttgagctgctgcatgcccctgccaccgtctgc
ggacctaaaaaatctaccaatttagtgaaaaataagtgcgtgaat
tttaatttcaacggccttacgggcaccggcgtgctgactgagagc
aataagaaattcttaccatttcagcagttcggccgcgacatagct
gataccaccgatgcagttcgcgacccccagaccctggagatcctt
gacatcactccttgcagtttcggaggagtctcggtcatcacacct
ggaacaaacacatccaaccaggtggcagttctttaccaggatgtg
aactgtaccgaagtgccagtggcaatccacgccgatcagttaact
cccacctggagagtgtactctacaggctctaacgtcttccagact
cgggccggttgccttattggggctgagcacgtgaacaactcctac
gagtgcgacatacctattggtgccggcatctgtgccagctaccag
acccaaaccaattcgccaaggcgagcgcgttctgtagcaagccag
tcgattattgcctacactatgtccttaggtgctgagaactctgtg
gcttactctaacaactccatagcaattccaacaaactttacaatt
agtgttactactgaaatcttgcccgtcagcatgactaaaacctct
gtcgattgtaccatgtacatttgtggggactctacagagtgcagc
aatcttctgctccagtacggctctttttgtacgcagctgaaccgt
gctctgactgggattgccgtcgagcaagataagaacacccaggag
gtgtttgcccaagttaagcagatttataagacaccacccatcaaa
gacttcggcggatttaacttttctcagattctgcccgacccctcc
aagcccagcaagaggagctttatcgaggacctgctgttcaataag
gtcacactcgctgatgcaggattcatcaagcagtatggcgattgc
ctcggagacatcgctgcgagagacctcatatgcgctcagaaattc
aatggcctgacggtgctacctccgctactgactgacgaaatgata
gctcagtacacgtcggctctcttggccggaacaatcacctccgga
tggacctttggcgcgggagcagcactacagatcccttttgcaatg
caaatggcttaccggttcaatggcataggggtaactcaaaatgtg
ctgtacgagaaccaaaaattgatcgctaaccagttcaacagcgcc
attgggaagatccaggattctttgtcctcaaccgccagtgcattg
ggcaagctccaggacgtcgttaaccagaacgctcaggccttaaac
acgctggtcaaacagttgtcctccaattttggcgctatatccagc
gttctcaatgatatcctttcccgcttagatccaccagaagctgag
gtccaaattgataggttaataaccggcagactccagagcctgcaa
acttacgtcacccagcaactcatacgcgccgcggagatccgcgct
agcgcaaacctagctgccactaaaatgagtgagtgtgttctcgga
cagtctaagcgggtggacttttgcggcaaaggctatcacctcatg
agcttcccccaaagcgcaccacatggcgttgtgttcttgcacgtg
acttacgttcccgcacaggagaaaaatttcaccacagcccccgcc
atctgccatgatgggaaagctcattttccacgagaaggggtgttc
gtgtcaaacggtacacactggtttgtcacacaaagaaatttttat
gaacctcaaattatcacaactgacaataccttcgtgagcggaaac
tgtgatgtcgtaattgggatcgtaaacaacactgtgtatgacccc
cttcagcccgaactggacagtttcaaggaagagcttgacaagtat
ttcaagaaccatacttcaccagacgtagaccttggtgatatttca
ggaatcaacgctagtgtggtgaacatccagaaagaaattgatcgc
ctcaatgaggtcgcgaaaaatctgaatgagtctctgatcgacctg
caagagttggggaagtacgaacagtatattaaatggccctggtac
atttggctgggatttatagctggactcattgccattgttatggtc
acaataatgctgtgttgcatgactagctgttgctcatgcctaaaa
gggtgctgcagttgcggctcctgttgcaagtttgatgaagacgat
agcgagccggtccttaaaggcgttaagctacattatactagggct
aagagatccggcagtggggcgaccaactttagcttgttgaagcaa
gcaggggacgtggaagaaaaccccggcccttacgccctagtgtac
tttctgcagtccattaacttcgttcggattatcatgaggttgtgg
ctgtgttggaagtgtcggtcgaagaatccactcctgtacgatgca
aattactttctgtgctggcacggagacggcaaaatgaaagacctg
tccccgagatggtatttttattatctgggtaccggtcccgaggcg
gggctgccctacggggcaaacaaagacggaatcatctgggtcgca
acagagggagctcttaatacacctaaagatcacattggcacccgc
aatcccgagaagtactgtgccctggcccccaatatgatggtgaca
aataacacctttacattaaagggcggggccccaaccaaggtgaca
ttcggtacatacaagaatacctgtgacggcacaacgttcacgtat
gcaagcgctctgtgggagatccaacaggtggtggacgccgacctg
aatgaaagtctgattgatctccaggaactcggcaaatatgagcag
tatatcaagtggccttggtacatctggctcggttttatcgctggt
cttatcgccatcgtgatggtgcagactgcttgcaccgatgataat
gcactcgcgtactacaacaccacaaaaggaggacgatttgtgcta
gccctgctcagtgatctgcaagatctcaaatgggcccgcttcccg
aagtccgatggaaccggcacaatctatacagaattggaacctcct
tgtaggttcgtgaccgatactcccaagggtcccaaggtaaaatac
ctgcgggtagaagcttttgaatactaccacactactgacccatct
tttctgggcagatatatgtctgcattaaatcacaccaaaaagtgg
ataacagtggccacctcccggacactgtcatactataaactgggt
gcatcccagcgggttgctggtgattccggattcgccgcctattcg
cggtatagaatagggaattacaagttgaataccgaccactccagt
tctagtgataacatagccctgctggttcaggttcttcagcagctg
agagtagaatcttccagcaagctgtgggcccagtgtgttcaactc
cacaatgatattttactcgccaaggacactgcaccgtcagcctct
gccttcttcgggatgtctcgtattggtatggaggttactcctagc
ggcacatggctgacgtacaccggggctataaagttggacgacaag
gacccaaacttcaaggaccaagtgatcttactgaacaaacatatc
gatgcttataagacattccctcctactgagcctaaaaaagataaa
tcaaagctcattgagtacacagattttgctacaagcgcttgtgtc
ctggcggccgagtgcaccatcttcaaagacgctagtggcaagccc
gtgccgtattgctatgacaccaatgtgctcgagggttcagtcgcc
tatgagtcattaaggccagatacgaggtacgtcctaatggatggg
TAG
>YP_009724389 (SARS-COV-2 ORF1a/b protein)
>YP_009724390 (SARS-COV-2 S protein)
>YP_009724397 (SARS-COV-2 N protein)
>YP_009724391 (SARS-COV-2 orf3a protein)
> YP_009724393.1 (SARS-COV-2 M protein)
> YP_009724395.1 (SARS-COV-2 orf7a protein)
*Included in Table 2 are nucleic acid sequences with at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity, or any range in between, inclusive, such as 85-99% identity, across their full length with a nucleic acid encoding a polypeptide listed in any one of Tables 1A-1K, or a portion thereof. Such nucleic acid sequences may encode polypeptides having one or more functions of the full-length peptide or polypeptide as described further herein and may also represent RNA nucleic acid molecules (e.g., thymines replaced with uredines).

In some embodiments, provided herein are orf1a/b polypeptides and/or nucleic acids encoding orf1a/b polypeptides. Orf1a/b polypeptides are polypeptides that include an amino acid sequence that corresponds to the amino acid sequence of an orf1a/b polyprotein, and/or a portion of the orf1a/b amino acid sequence of sufficient length to elicit an orf1a/b-specific immune response. In certain embodiments, the orf1a/b polypeptide also includes amino acids that do not correspond to the amino acid sequence (e.g., a fusion protein comprising an orf1a/b amino acid sequence and an amino acid sequence corresponding to a non-orf1a/b protein or polypeptide). In some embodiments, the orf1a/b polypeptide only includes amino acid sequence corresponding to an orf1a/b polyprotein or fragment thereof.

In some embodiments, the orf1a/b polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 95, 100, 200, 300, 400, 500, 600, 700, 800, 900, 1000, 1500, 2000, 2500, 3000, 3500, 4000, 4500, 5000, 5500, 6000, 6500, or 7000 consecutive amino acids of an orf1a/b protein amino acid sequence set forth in Table 1K. In some embodiments, the consecutive amino acids are identical to an amino acid sequence of orf1a/b set forth in Table 1K. In some embodiments, orf1a/b polypeptides comprise, consist essentially of, or consist of one or more peptide epitopes selected from the group consisting of orf1a/b peptide epitopes listed in Table 1A, 1B, 1C, 1D, 1E, and/or 1F.

In some embodiments, provided herein are S protein polypeptides and/or nucleic acids encoding S protein polypeptides. S protein polypeptides are polypeptides that include an amino acid sequence that corresponds to the amino acid sequence of an S protein polyprotein, and/or a portion of the S protein amino acid sequence of sufficient length to elicit an S protein-specific immune response. In certain embodiments, the S protein polypeptide also includes amino acids that do not correspond to the amino acid sequence (e.g., a fusion protein comprising an S protein amino acid sequence and an amino acid sequence corresponding to a non-S protein or polypeptide). In some embodiments, the S protein polypeptide only includes amino acid sequence corresponding to an S protein polyprotein or fragment thereof.

In certain embodiments, the S protein polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 95, 100, 150, 200, 250, 300, 350, 400, 450, 500, 550, 600, 650, 700, 750, 800, 850, 900, 950, 1000, 1050, 1100, 1150, 1200, or 1250 consecutive amino acids of an S protein amino acid sequence set forth in Table 1K. In some embodiments, the consecutive amino acids are identical to an amino acid sequence of S protein set forth in Table 1K. In some embodiments, S polypeptides comprise, consist essentially of, or consist of one or more peptide epitopes selected from the group consisting of S peptide epitopes listed in Table 1A, 1B, 1C, 1D, 1E, and/or 1F.

In some embodiments, provided herein are N protein polypeptides and/or nucleic acids encoding N protein polypeptides. N protein polypeptides are polypeptides that include an amino acid sequence that corresponds to the amino acid sequence of an N protein polyprotein, and/or a portion of the N protein amino acid sequence of sufficient length to elicit an N protein-specific immune response. In certain embodiments, the N protein polypeptide also includes amino acids that do not correspond to the amino acid sequence (e.g., a fusion protein comprising an N protein amino acid sequence and an amino acid sequence corresponding to a non-N protein or polypeptide). In some embodiments, the N protein polypeptide only includes amino acid sequence corresponding to an N protein polyprotein or fragment thereof.

In certain embodiments, the N protein polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 95, 100, 110, 120, 130, 140, 150, 160, 170, 180, 190, 200, 210, 220, 230, 240, 250, 260, 270, 280, 290, or 300 consecutive amino acids of an N protein amino acid sequence set forth in Table 1K. In some embodiments, the consecutive amino acids are identical to an amino acid sequence of an N protein set forth in Table 1K. In some embodiments, N polypeptides comprise, consist essentially of, or consist of one or more peptide epitopes selected from the group consisting of N peptide epitopes listed in Table 1A, 1B, 1C, 1D, 1E, and/or 1F.

In some embodiments, provided herein are M protein polypeptides and/or nucleic acids encoding M protein polypeptides. M protein polypeptides are polypeptides that include an amino acid sequence that corresponds to the amino acid sequence of an M protein polyprotein, and/or a portion of the M protein amino acid sequence of sufficient length to elicit an M protein-specific immune response. In certain embodiments, the M protein polypeptide also includes amino acids that do not correspond to the amino acid sequence (e.g., a fusion protein comprising an M protein amino acid sequence and an amino acid sequence corresponding to a non-M protein or polypeptide). In some embodiments, the M protein polypeptide only includes amino acid sequence corresponding to an N protein polyprotein or fragment thereof.

In certain embodiments, the M protein polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 95, 100, 110, 120, 130, 140, 150, 160, 170, 180, 190, 200, 210, or 220 consecutive amino acids of an M protein amino acid sequence set forth in Table 1K. In some embodiments, the consecutive amino acids are identical to an amino acid sequence of an M protein set forth in Table 1K. In some embodiments, M polypeptides comprise, consist essentially of, or consist of one or more peptide epitopes selected from the group consisting of M peptide epitopes listed in Table 1A, 1B, 1C, 1D, 1E, and/or 1F.

In some embodiments, provided herein are orf3a polypeptides and/or nucleic acids encoding orf3a polypeptides. Orf3a polypeptides are polypeptides that include an amino acid sequence that corresponds to the amino acid sequence of an orf3a polyprotein, and/or a portion of the orf3a amino acid sequence of sufficient length to elicit an orf3a-specific immune response. In certain embodiments, the orf3a polypeptide also includes amino acids that do not correspond to the amino acid sequence (e.g., a fusion protein comprising an orf3a amino acid sequence and an amino acid sequence corresponding to a non-orf3a protein or polypeptide). In some embodiments, the orf3a polypeptide only includes amino acid sequence corresponding to an orf3a polyprotein or fragment thereof.

In certain embodiments, the orf3a polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 95, 100, 110, 120, 130, 140, 150, 160, 170, 180, 190, 200, 210, 220, 230, 240, 250, 260, or 270 consecutive amino acids of an orf3a amino acid sequence set forth in Table 1K. In some embodiments, the consecutive amino acids are identical to an amino acid sequence of an orf3a protein set forth in Table 1K. In some embodiments, orf3a polypeptides comprise, consist essentially of, or consist of one or more peptide epitopes selected from the group consisting of orf3a peptide epitopes listed in Table 1A, 1B, 1C, 1D, 1E, and/or 1F.

In some embodiments, provided herein are orf7a polypeptides and/or nucleic acids encoding orf7a polypeptides. Orf7a polypeptides are polypeptides that include an amino acid sequence that corresponds to the amino acid sequence of an orf7a polyprotein, and/or a portion of the orf7a amino acid sequence of sufficient length to elicit an orf7a-specific immune response. In certain embodiments, the orf7a polypeptide also includes amino acids that do not correspond to the amino acid sequence (e.g., a fusion protein comprising an orf7a amino acid sequence and an amino acid sequence corresponding to a non-orf7a protein or polypeptide). In some embodiments, the orf7a polypeptide only includes amino acid sequence corresponding to an orf7a polyprotein or fragment thereof.

In certain embodiments, the orf7a polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, or 120 consecutive amino acids of an orf7a amino acid sequence set forth in Table 1K. In some embodiments, the consecutive amino acids are identical to an amino acid sequence of an orf7a protein set forth in Table 1K. In some embodiments, orf7a polypeptides comprise, consist essentially of, or consist of one or more peptide epitopes selected from the group consisting of orf7a peptide epitopes listed in Table 1A, 1B, 1C, 1D, 1E, and/or 1F.

As is well-known to those skilled in the art, polypeptides having substantial sequence similarities can cause identical or very similar immune reaction in a host animal. Accordingly, in some embodiments, a derivative, equivalent, variant, fragment, or mutant of a SARS-CoV-2 immunogenic peptide described herein or fragment thereof may also suitable for the methods and compositions provided herein.

In some embodiments, variations or derivatives of the SARS-CoV-2 immunogenic polypeptides are provided herein. The altered polypeptide may have an altered amino acid sequence, for example by conservative substitution, yet still elicits immune responses which react with the unaltered protein antigen, and are considered functional equivalents. As used herein, the term “conservative substitution” denotes the replacement of an amino acid residue by another, biologically similar residue. It is well known in the art that the amino acids within the same conservative group may typically substitute for one another without substantially affecting the function of a protein. According to certain embodiments, the derivative, equivalents, variants, or mutants of the ligand-binding domain of a SARS-CoV-2 immunogenic peptide are polypeptides that are at least 85% homologous to the sequence of a SARS-CoV-2 immunogenic peptide described herein or fragment thereof. In some embodiments, the homology is at least 90%, at least 95%, at least 98%, or more.

Immunogenic peptides encompassed by the present invention may comprise a peptide epitope derived from a SARS-CoV-2 protein, such as those listed in Table 1A, 1B, 1C, 1D, 1E, and/or 1F. In some embodiments, the immunogenic peptide is 8, 9, 10, 11, 12, 13, 14, or 15 amino acids in length. In some embodiments, the peptide amino acid sequences is modified, which may include conservative or non-conservative mutations. A peptide may comprise at most 1, 2, 3, 4, or more mutations. In some embodiments, a peptide may comprise at least 1, 2, 3, 4, or more mutations.

In some embodiments, a peptide may be chemically modified. For example, a peptide can be mutated to modify peptide properties such as detectability, stability, biodistribution, pharmacokinetics, half-life, surface charge, hydrophobicity, conjugation sites, pH, function, and the like. N-methylation is one example of methylation that can occur in a peptide encompassed by the present invention. In some embodiments, a peptide may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a peptide with an Fc region may be a fusion Fc-peptide. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing. In some embodiments, the peptides encompassed by the present invention may be modified such that the modification increases the stability and/or the half-life of the peptides. In some embodiments, the attachment of a hydrophobic moiety, such as to the N-terminus, the C-terminus, or an internal amino acid, can be used to extend half-life of a peptide encompassed by the present invention. In other embodiments, a peptide may include post-translational modifications (e.g., methylation and/or amidation), which can affect, for example, serum half-life. In some embodiments, simple carbon chains (e.g., by myristoylation and/or palmitylation) can be conjugated to the fusion proteins or peptides. In some embodiments, the simple carbon chains may render the fusion proteins or peptides easily separable from the unconjugated material. For example, methods that may be used to separate the fusion proteins or peptides from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the peptides may be conjugated to myristic acid (tetradecanoic acid) or a derivative thereof. In other embodiments, a peptide may be coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a peptide, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, fusion proteins or peptides may be conjugated to other moieties that, for example, can modify or effect changes to the properties of the peptides.

A peptide may be conjugated to an agent used in imaging, research, therapeutics, theranostics, pharmaceuticals, chemotherapy, chelation therapy, targeted drug delivery, and radiotherapy. In some embodiments, a peptide may be conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, 10, or more detectable moieties may be linked to a peptide. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that may be used as a conjugating molecule include DyLight-680, DyLight-750, VivoTag-750, DyLight-800, IRDye-800, VivoTag-680, Cy5.5, ZQ800, or indocyanine green (ICG). In some embodiments, near infrared dyes often include cyanine dyes (e.g., Cy7, Cy5.5, and Cy5). Additional non-limiting examples of fluorescent dyes for use as a conjugating molecule encompassed by the present invention include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

A peptide may be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cells of interest (e.g., immune cells) using both a therapeutic agent (e.g., drug) and electromagnetic energy (e.g., radiation or light) concurrently. In some embodiments, the peptide is fused with, or covalently or non-covalently linked to the agent, for example, directly or via a linker.

A peptide may be produced recombinantly or synthetically, such as by solid-phase peptide synthesis or solution-phase peptide synthesis. Peptide synthesis may be performed by known synthetic methods, such as using fluorenylmethyloxycarbonyl (Fmoc) chemistry or by butyloxycarbonyl (Boc) chemistry. Peptide fragments may be joined together enzymatically or synthetically.

In some embodiments, provided herein is a nucleic acid encoding a SARS-CoV-2 immunogenic polypeptide described herein or fragment thereof, such as a DNA molecule encoding a SARS-CoV-2 immunogenic peptide. In some embodiments, the composition comprises an expression vector comprising an open reading frame encoding a SARS-CoV-2 immunogenic peptide described herein or fragment thereof. In some embodiments, the nucleic acid includes regulatory elements necessary for expression of the open reading frame. Such elements may include, for example, a promoter, an initiation codon, a stop codon, and a polyadenylation signal. In addition, enhancers may be included. These elements may be operably linked to a sequence that encodes the SARS-CoV-2 immunogenic polypeptide or fragment thereof.

Examples of promoters include but are not limited to promoters from Simian Virus (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from human genes such as human actin, human myosin, human hemoglobin, human muscle creatine, and human metalothionein. Examples of suitable polyadenylation signals include but are not limited to SV40 polyadenylation signals and LTR polyadenylation signals.

In addition to the regulatory elements required for expression, other elements may also be included in the nucleic acid molecule. Such additional elements include enhancers. Enhancers include the promoters described hereinabove. Preferred enhancers/promoters include, for example, human actin, human myosin, human hemoglobin, human muscle creatine and viral enhancers such as those from CMV, RSV and EBV.

In some embodiments, the nucleic acid may be used alone (e.g., as naked nucleic acids) or operably incorporated in a carrier or delivery vector as described further below. Useful delivery vectors include but are not limited to biodegradable microcapsules, immuno-stimulating complexes (ISCOMs) or liposomes, and genetically engineered attenuated live carriers such as viruses or bacteria.

In some embodiments, the vector is a viral vector, such as lentiviruses, retroviruses, herpes viruses, adenoviruses, adeno-associated viruses, vaccinia viruses, baculoviruses, Fowl pox, AV-pox, modified vaccinia Ankara (MVA) and other recombinant viruses. For example, a lentivirus vector may be used to infect T cells.

### III. Nucleic Acids, Vectors, and Cells

A further object of the present invention relates to nucleic acid sequences encoding the described immunogenic polypeptides, SARS-CoV-2 immunogenic peptides and fragments thereof, MHC molecules, and TCRs and fragments thereof. In some aspects, disclosed herein are nucleic acid vector constructs that maximize vector size to T cell response efficacy by packing in optimal immunodominant epitopes for intracellular antigen expression. Generally, as described further below, nucleic acids encompassed by the present invention, whether naked or incorporated within a vector, may be direct vaccine constructs (or vectors used to make them), including mRNA (or in vitro transcription expression vector generating mRNA), mammalian expression vector for use as a DNA vaccine, and the like. Nucleic acids encompassed by the present invention may be codon-optimized for certain purposes, such as high expression in human subjects. Nucleic acids encompassed by the present invention may be engineered to have high guanine and cytosine (G-C) content, such as at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity, or any range in between, inclusive, such as 60-70% G-C content.

In a particular embodiment, the present invention relates to a nucleic acid sequence encoding the SARS-CoV-2 immunogenic peptides described herein. In a particular embodiment, the present invention relates to a nucleic acid sequence encoding the immunogenic polypeptides described herein.

Typically, said nucleic acid is a DNA (e.g., cDNA) or RNA (e.g., mRNA) molecule, which may be basic or included in any suitable vector, such as a plasmid, cosmid, episome, artificial chromosome, phage, virus, or a viral vector.

Such basic nucleic acid may be a “primary construct” such as a primary mRNA construct, which refers to a polynucleotide transcript which encodes one or more polypeptides of interest and which retains sufficient structural and/or chemical features to allow the polypeptide of interest encoded therein to be translated. Primary constructs may be polynucleotides encompassed by the present invention. When structurally or chemically modified, the primary construct may be referred to as a modified nucleic acid, such as a modified mRNA. Nucleic acid constructs may comprise sequences in addition to polypeptide encoding sequences, such as capping sequences, tailing sequences, cyclization sequences, and the like, which are well-known in the art. For example, tailing sequence may range from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional. A capping region may comprise a single cap or a series of nucleotides forming the cap. In this embodiment the capping region may be from 1 to 10 (e.g., 2-9, 3-8, 4-7, 1-5, 5-10, or at least 2, or 10 or fewer nucleotides in length). In some embodiments, the cap is absent. Thus, nucleic acids encompassed by the present invention may thus comprise protein-encoding regions from 2 to 40 or more, e.g., 2-19, 2-28, etc., and may further comprise one or more additional elements described herein, such as start and/or stop codons, translation sequences, internal ribosomal entry sequences, protein cleavage sequences, signal sequences, capping sequences, tailing sequences, restriction sequences, self-replication sequences, and the like. In some embodiments, nucleic acids encompassed by the present invention may be cyclized and/or concatemerized, such as through chemical, enzymatic, and/or ribozyme catalyzed means well-known in the art. Newly formed 5′-/3′-linkage may be intramolecular or intermolecular.

In some embodiments, nucleic acids encompassed by the present invention are self-replicating, such as self-replicating RNA like mRNA. RNA is cost-effective to produce in large quantities and can be generated endotoxin-free from any given discovered sequence from commercially synthesized DNA precursors with nearly same-day rapidity. It is generally safer and easier to administer than DNA because it does not pose a risk of genome integration, and only requires access to cell cytoplasm to function. Moreover, due to the availability of self-replicating RNAs (repRNAs) based on alphavirus or flavivirus genomes, very low doses can be employed to achieve maximal immunogenicity and antigen production levels. In some embodiments, repRNAs are attenuated virus genomes lacking viral structural proteins required for the production of progeny virions but retain the capability of translation and replication and can therefore effectively increase the half-life of translation of RNA. For example, delivery of RepRNAs encoding one or more exogenous genes to cells can effectively increase the translation and expression of the exogenous genes in the cells relative to that resulting from delivery of an equal molar amount of conventional mRNAs encoding the one or more exogenous genes to the cells. In some embodiments, the RepRNAs are non-cyto-pathogenic RepRNAs. In some embodiments, the repRNA is an alphaviral self-amplifying repRNA (e.g., including a 5′ cap; 5′ untranslated region (5′ UTR), non-structural genes (e.g., NSP1-4) encoded within a first open reading frame, a genomic promoter region (e.g., 26S sub-genomic promoter), a second open reading frame, a 3′ untranslated region (3′ UTR) and a 3′ poly-adenylated tail. repRNA molecules are typically between 9,000 and 20,000 nucleotides in length, depending upon the size of the encoded genic sequence). The non-structural genes encode an RNA-dependent RNA polymerase (RdRp). Typically, the RdRp does not tolerate classical nucleotide modifications that are used to protect conventional RNA against endonucleose and autocatalytic degradation. Thus, nano-encapsulation (e.g., nanoparticles, lipids, lipid nanoparticles, cationic molecules, polymers, and the like) may be used for the effective deployment in expression platforms. repRNA is modular and open reading frames can be engineered to accommodate an exogenous sequence(s) of interest. When the repRNA is deposited into the cytoplasm of a host cell, the RNA dependent RNA polymerase (RdRp) encoded by the repRNA NS genes is expressed within the cell. The RdRp can then replicate the entire repRNA, or the RdRp copies of the repRNA-encoded antigen only (i.e., by virtue of a sub-genomic promoter). Replicon RNAs increase the overall efficiency of RNA-mediated gene delivery, because the repRNA can synthesize more copies of the full-length replicon, as well as more copies of mRNAs encoding the genes included within the second open reading frame. The host cell ribosomes continue to translate the full-length replicon copies or the shorter antigen-only mRNAs, leading to enhanced expression of the genes encoded by the repRNA.

In order to further enhance protein production, nucleic acids encompassed by the present invention may be designed to be conjugated to other polynucleotides, dyes, intercalating agents (e.g., acridines), cross-linkers (e.g., psoralene and mitomycin C), porphyrins (TPPC4, texaphyrin, and sapphyrin), polycyclic aromatic hydrocarbons (e.g., phenazine and dihydrophenazine), artificial endonucleases (e.g., EDTA), alkylating agents, phosphate, amino, mercapto, PEG (e.g., PEG-40K), MPEG, MPEG2, polyamino, alkyl, substituted alkyl, radiolabeled markers, enzymes, haptens (e.g., biotin), transport/absorption facilitators (e.g., aspirin, vitamin E, and folic acid), synthetic ribonucleases, proteins (e.g., glycoproteins), or peptides (e.g., molecules having a specific affinity for a co-ligand), or antibodies (e.g., an antibody, that binds to a specified cell type such as a cancer cell, endothelial cell, or bone cell), hormones and hormone receptors, and/or non-peptidic species (e.g., lipids, lectins, carbohydrates, vitamins, cofactors, and a drug). Representative examples of nucleic acids encompassed by the present invention are described herein, such as in the working examples and figures, and are well-known in the art (see, at least U.S. Pat. Publ. 2020/0354423, U.S. Pat. Publ. 2020/0254086, and U.S. Pat. Publ. 2020/0155660).

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Thus, a further object encompassed by the present relates to a vector comprising a nucleic acid encompassed by the present invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478. In some embodiments, viral vector-based platforms may be used. Representative, non-limiting examples include vaccinia, fowlpox, self-replicating alphavirus, marabavirus, adenovirus, and lentivirus, including, but not limited to, second, third, or hybrid second/third generation lentivirus and recombinant lentivirus of any generation designed to target specific cell types or receptors (see, at least Hu et al. (2011) Immunol Rev. 239:45-61, Sakuma et al. (2012) Biochem J. 443:603-618, Cooper et al. (2015) Nucl. Acids Res. 43:682-690, Zufferey et al. (1998) J Virol. 72:9873-9880, and U.S. Pat. Publ. 2020/0010849).

A further object of the present invention relates to a cell which has been transfected, infected or transformed by a nucleic acid and/or a vector according to the present invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids encompassed by the present invention may be used to produce a recombinant polypeptide encompassed by the present in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

The present invention also relates to a method of producing a recombinant host cell expressing SARS-CoV-2 immunogenic peptides and fragments thereof, MHC molecules, and TCRs and fragments thereof encompassed by the present according to the present invention, said method comprising the steps consisting of (i) introducing in vitro or ex vivo a recombinant nucleic acid or a vector as described above into a competent host cell, (ii) culturing in vitro or ex vivo the recombinant host cell obtained and (iii), optionally, selecting the cells which express said SARS-CoV-2 immunogenic peptides and fragments thereof, MHC molecules, and TCRs and fragments thereof. Such recombinant host cells can be used for the diagnostic, prognostic, and/or therapeutic method encompassed by the present.

In another aspect, the present invention provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides encompassed by the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences. Optionally, polynucleotides encompassed by the present invention will encode at least a portion of an antibody encoded by the polynucleotides described herein. The polynucleotides encompassed by the present invention embrace nucleic acid sequences that can be employed for selective hybridization to a polynucleotide encoding an antibody encompassed by the present invention. See, e.g., Ausubel, supra; Colligan, supra, each entirely incorporated herein by reference.

### IV. MHC-Peptide Complexes

In certain aspects, provided herein are compositions comprising a SARS-CoV-2 immunogenic peptide described herein and a MHC molecule. In some embodiments, the SARS-CoV-2 immunogenic peptide forms a stable complex with the MHC molecule.

The MHC proteins provided and used in the compositions and methods encompassed by the present invention may be any suitable MHC molecules known in the art. Generally, they have the formula (α-β-P)ₙ, where n is at least 2, for example between 2-10, e.g., 4. α is an α chain of a class I or class II MHC protein. β is a β chain, herein defined as the β chain of a class II MHC protein or β₂ microglobulin for a MHC class I protein. P is a peptide antigen.

In some embodiments, the MHC proteins are MHC class I complexes, such as HLA I complexes.

The MHC proteins may be from any mammalian or avian species, e.g., primate sp., particularly humans; rodents, including mice, rats and hamsters; rabbits; equines, bovines, canines, felines; etc. For instance, the MHC protein may be derived the human HLA proteins or the murine H-2 proteins. HLA proteins include the class II subunits HLA-DPα, HLA-DPβ, HLA-DQα, HLA-DQβ, HLA-DRα and HLA-DRβ, and the class I proteins HLA-A, HLA-B, HLA-C, and β2-microglobulin. H-2 proteins include the class I subunits H-2K, H-2D, H-2L, and the class II subunits I-Aα, I-Aβ, I-Eα and I-Eβ, and β2-microglobulin. Sequences of some representative MHC proteins may be found in Kabat et al. Sequences of Proteins of Immunological Interest, NIH Publication No. 91-3242, pp 724-815. MHC protein subunits suitable for use according to the present invention are a soluble form of the normally membrane-bound protein, which is prepared as known in the art, for instance by deletion of the transmembrane domain and the cytoplasmic domain.

For class I proteins, the soluble form may include the α1, α2 and α3 domain. Soluble class II subunits may include the α1 and α2 domains for the a subunit, and the β1 and β2 domains for the β subunit.

The α and β subunits may be separately produced and allowed to associate in vitro to form a stable heteroduplex complex, or both of the subunits may be expressed in a single cell. Methods for producing MHC subunits are known in the art.

In certain embodiments, the MHC-peptide complex comprises a peptide epitope selected from Table 1A and an MHC whose alpha chain has an HLA-A*02 serotype, such as that encoded by an HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, and/or HLA-A*0274 allele. In other embodiments, the MHC-peptide complex comprises a peptide epitope selected from Table 1C and an MHC whose alpha chain has an HLA-A*03 serotype, such as that encoded by an HLA-A*0301, HLA-A*0302, HLA-A*0305, and/or HLA-A*0307 allele. In still other embodiments, the MHC-peptide complex comprises a peptide epitope selected from Table 1B and an MHC whose alpha chain has an HLA-A*01 serotype, such as that encoded by an HLA-A*0101, HLA-A*0102, HLA-A*0103, and/or HLA-A*0116 allele. In yet other embodiments, the MHC-peptide complex comprises a peptide epitope selected from Table 1D and an MHC whose alpha chain has an HLA-A*11 serotype, such as that encoded by an HLA-A*1101, HLA-A*1102, HLA-A*1103, HLA-A*1104, HLA-A*1105, and/or HLA-A*1119 allele. In other embodiments, the MHC-peptide complex comprises a peptide epitope selected from Table 1E and an MHC whose alpha chain has an HLA-A*24 serotype, such as that encoded by an HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, and/or HLA-A*2458 allele. In still other embodiments, the MHC-peptide complex comprises a peptide epitope selected from Table 1F and an MHC whose alpha chain has an HLA-B*07 serotype, such as that encoded by an HLA-B*0702, HLA-B*0704, HLA-B*0705, HLA-B*0709, HLA-B*0710, HLA-B*0715, and/or HLA-B*0721 allele.

To prepare the MHC-peptide complex, the subunits may be combined with an antigenic peptide and allowed to fold in vitro to form a stable heterodimer complex with intrachain disulfide bonded domains. The peptide may be included in the initial folding reaction, or may be added to the empty heterodimer in a later step. In the compositions and methods encompassed by the present invention, this is a SARS-CoV-2 immunogenic peptide or fragment thereof. Conditions that permit folding and association of the subunits and peptide are known in the art. As one example, roughly equimolar amounts of solubilized α and β subunits may be mixed in a solution of urea. Refolding is initiated by dilution or dialysis into a buffered solution without urea. Peptides may be loaded into empty class II heterodimers at about pH 5 to 5.5 for about 1 to 3 days, followed by neutralization, concentration and buffer exchange. However, the specific folding conditions are not critical for the practice of the present invention.

The monomeric complex (α-β-P) (herein monomer) may be multimerized, for example, for a MHC tetramer. The resulting multimer is stable over long periods of time. Preferably, the multimer may be formed by binding the monomers to a multivalent entity through specific attachment sites on the α or β subunit, as known in the art (e.g., as described in U.S. Pat. No. 5,635,363). The MHC proteins, in either their monomeric or multimeric forms, may also be conjugated to beads or any other support.

The multimeric complex may be labeled, so as to be directly detectable when used in immunostaining or other methods known in the art, or may be used in conjunction with secondary labeled immunoreagents which specifically bind the complex (e.g., bind to a MHC protein subunit) as known in the art. For example, the detectable label may be a fluorophore, such as fluorescein isothiocyanate (FITC), rhodamine, Texas Red, phycoerythrin (PE), allophycocyanin (APC), Brilliant Violet™ 421, Brilliant UV™ 395, Brilliant Violet™ 480, Brilliant Violet™ 421 (BV421), Brilliant Blue™ 515, APC-R700, or APC-Fire750. In some embodiments, the multimeric complex is labeled by a moiety that is capable of specifically binding another moiety. For instance, the label may be biotin, streptavidin, an oligonucleotide, or a ligand. Other labels of interest may include fluorochromes, dyes, enzymes, chemiluminescers, particles, radioisotopes, or other directly or indirectly detectable agent.

In some embodiments, a cell presenting an immunogenic peptides in context of an MHC molecule on the cell surface is generated by transfecting or transducing the cell with a vector (e.g., a viral vector) that comprising nucleic acid that encodes a recombinant or heterologous antigen into a cell. In some embodiments, the vector is introduced into the cell under conditions in which one or more peptide antigens, including, in some cases, one or more peptide antigens of the expressed heterologous protein, are expressed by the cell, processed and presented on the surface of the cell in the context of a major histocompatibility complex (MHC) molecule.

Generally, the cell to which the vector is contacted is a cell that expresses MHC, i.e., MHC-expressing cells. The cell may be one that normally expresses an MHC on the cell surface, that is induced to express and/or upregulate expression of MHC on the cell surface or that is engineered to express an MHC molecule on the cell surface. In some embodiments, the MHC contains a polymorphic peptide binding site or binding groove that can, in some cases, complex with peptide antigens of polypeptides, including peptide antigens processed by the cell machinery. In some cases, MHC molecules may be displayed or expressed on the cell surface, including as a complex with peptide, i.e., MHC-peptide complex, for presentation of an antigen in a conformation recognizable by TCRs on T cells, or other peptide binding molecules.

In some embodiments, the cell is a nucleated cell. In some embodiments, the cell is an antigen-presenting cell. In some embodiments, the cell is a macrophage, dendritic cell, B cell, endothelial cell or fibroblast. In some embodiments, the cell is an endothelial cell, such as an endothelial cell line or primary endothelial cell. In some embodiments, the cell is a fibroblast, such as a fibroblast cell line or a primary fibroblast cell.

In some embodiments, the cell is an artificial antigen presenting cell (aAPC). Typically, aAPCs include features of natural APCs, including expression of an MHC molecule, stimulatory and costimulatory molecule(s), Fc receptor, adhesion molecule(s) and/or the ability to produce or secrete cytokines (e.g., IL-2). Normally, an aAPC is a cell line that lacks expression of one or more of the above, and is generated by introduction (e.g., by transfection or transduction) of one or more of the missing elements from among an MHC molecule, a low affinity Fc receptor (CD32), a high affinity Fc receptor (CD64), one or more of a co-stimulatory signal (e.g., CD7, B7-1 (CD80), B7-2 (CD86), PD-L1, PD-L2, 4-1BBL, OX40L, ICOS-L, ICAM, CD30L, CD40, CD70, CD83, HLA-G, MICA, MICB, HVEM, lymphotoxin beta receptor, ILT3, ILT4, 3/TR6 or a ligand of B7-H3; or an antibody that specifically binds to CD27, CD28, 4-1BB, OX40, CD30, CD40, PD-1, ICOS, LFA-1, CD2, CD7, LIGHT, NKG2C, B7-H3, Toll ligand receptor or a ligand of CD83), a cell adhesion molecule (e.g., ICAM-1 or LFA-3) and/or a cytokine (e.g., IL-2, IL-4, IL-6, IL-7, IL-10, IL-12, IL-15, IL-21, interferon-alpha (IFNalpha), interferon-beta (IFNbeta), interferon-gamma (IFNgamma), tumor necrosis factor-alpha (TNFalpha), tumor necrosis factor-beta (TNFbeta), granulocyte macrophage colony stimulating factor (GM-CSF), and granulocyte colony stimulating factor (GCSF)). In some cases, an aAPC does not normally express an MHC molecule, but may be engineered to express an MHC molecule or, in some cases, is or may be induced to express an MHC molecule, such as by stimulation with cytokines. In some cases, aAPCs also may be loaded with a stimulatory ligand, which may include, for example, an anti-CD3 antibody, an anti-CD28 antibody or an anti-CD2 antibody. An exemplary cell line that may be used as a backbone for generating an aAPC is a K562 cell line or a fibroblast cell line. Various aAPCs are known in the art, see e.g., U.S. Pat. No. 8,722,400, published application No. US2014/0212446; Butler and Hirano (2014) Immunol Rev., 257(1):10. 1111/imr.12129; Suhoshki et al. (2007) Mol. Ther., 15:981-988).

It is well within the level of a skilled artisan to determine or identify the particular MHC or allele expressed by a cell. In some embodiments, prior to contacting cells with a vector, expression of a particular MHC molecule may be assessed or confirmed, such as by using an antibody specific for the particular MHC molecule. Antibodies to MHC molecules are known in the art, such as any described below.

In some embodiments, the cells may be chosen to express an MHC allele of a desired MHC restriction. In some embodiments, the MHC typing of cells, such as cell lines, are well known in the art. In some embodiments, the MHC typing of cells, such as primary cells obtained from a subject, may be determined using procedures well known in the art, such as by performing tissue typing using molecular haplotype assays (BioTest ABC SSPtray, BioTest Diagnostics Corp., Denville, N.J.; SeCore Kits, Life Technologies, Grand Island, N.Y.). In some cases, it is well within the level of a skilled artisan to perform standard typing of cells to determine the HLA genotype, such as by using sequence-based typing (SBT) (Adams et al. (2004) J. Transl. Med., 2:30; Smith (2012) Methods Mol Biol., 882:67-86). In some cases, the HLA typing of cells, such as fibroblast cells, are known. For example, the human fetal lung fibroblast cell line MRC-5 is HLA-A*0201, A29, B13, B44 Cw7 (C*0702); the human foreskin fibroblast cell line Hs68 is HLA-A1, A29, B8, B44, Cw7, Cw16; and the WI-38 cell line is A*6801, B*0801, (Solache et al. (1999) J Immunol, 163:5512-5518; Ameres et al. (2013) PloS Pathog. 9:e1003383). The human transfectant fibroblast cell line M1DR1/Ii/DM express HLA-DR and HLA-DM (Karakikes et al. (2012) FASEB J., 26:4886-96).

In some embodiments, the cells to which the vector is contacted or introduced are cells that are engineered or transfected to express an MHC molecule. In some embodiments, cell lines may be prepared by genetically modifying a parental cells line. In some embodiments, the cells are normally deficient in the particular MHC molecule and are engineered to express such particular MHC molecule. In some embodiments, the cells are genetically engineered using recombinant DNA techniques.

In some embodiments, the stable MHC-peptide complexes described herein are used to detect T cells that bind a stable MHC-peptide complex. In some embodiments, the stable MHC-peptide complexes described herein are used to monitor T cell response in a subject, for example, by detecting the amount and/or percentage of T cells (e.g., CD8+ T cells) that specifically bind to the MHC-peptide complexes that are fluorescently labeled. Methods of generating, labeling, and using MHC-peptide complexes (e.g., MHC-peptide tetramers) for detecting MHC-peptide complex-specific T cells are well known in the art. Additional description can be found in, for example, U.S. Pat. Nos. 7,776,562; 8,268,964; and U.S. Pat. Publ. No. 2019/0085048, each of which is incorporated herein by reference in its entirety.

### V. Immunogenic Compositions

In some aspects, provided herein are pharmaceutical compositions (e.g., a vaccine composition) comprising a SARS-CoV-2 immunogenic peptide and/or a nucleic acid encoding a SARS-CoV-2 immunogenic peptide and an adjuvant. In some aspects, provided herein are pharmaceutical compositions (e.g., a vaccine composition) comprising an immunogenic polypeptide and/or a nucleic acid encoding an immunogenic polypeptide and an adjuvant. In some aspects, provided herein are pharmaceutical compositions (e.g., a vaccine composition) comprising a stable MHC-peptide complex comprising a SARS-CoV-2 immunogenic peptide in the context of a MHC molecule and an adjuvant. In some embodiments, the composition includes a combination of multiple (e.g., two or more) SARS-CoV-2 immunogenic peptides or nucleic acids and an adjuvant. In some embodiments, the composition includes a combination of multiple (e.g., two or more) stable MHC-peptide complexes comprising a SARS-CoV-2 immunogenic peptide in the context of a MHC molecule and an adjuvant. In some embodiments, the compositions described above further comprises a pharmaceutically acceptable carrier.

The pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Methods of preparing these formulations or compositions include the step of bringing into association a SARS-CoV-2 immunogenic peptide and/or nucleic acid described herein with the adjuvant, carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise SARS-CoV-2 immunogenic peptides and/or nucleic acids described herein in combination with a adjuvant, as well as one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

In some embodiments, the pharmaceutical composition described, when administered to a subject, can elicit an immune response against a cell that is infected by SARS-CoV-2. Such pharmaceutical compositions may be useful as vaccine compositions for prophylactic and/or therapeutic treatment of COVID-19.

In some embodiments, the pharmaceutical composition further comprises a physiologically acceptable adjuvant. In some embodiments, the adjuvant employed provides for increased immunogenicity of the pharmaceutical composition. Such a further immune response stimulating compound or adjuvant may be (i) admixed to the pharmaceutical composition according to the present invention after reconstitution of the peptides and optional emulsification with an oil-based adjuvant as defined above, (ii) may be part of the reconstitution composition encompassed by the present defined above, (iii) may be physically linked to the peptide(s) to be reconstituted or (iv) may be administered separately to the subject, mammal or human, to be treated. The adjuvant may be one that provides for slow release of antigen (e.g., the adjuvant may be a liposome), or it may be an adjuvant that is immunogenic in its own right thereby functioning synergistically with antigens (i.e., antigens present in the SARS-CoV-2 immunogenic peptide). For example, the adjuvant may be a known adjuvant or other substance that promotes antigen uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, Bacto-Adjuvant, synthetic polymers such as poly amino acids and co-polymers of amino acids, saponin, paraffin oil, and muramyl dipeptide. In some embodiments, the adjuvant is Adjuvant 65, α-GalCer, aluminum phosphate, aluminum hydroxide, calcium phosphate, β-Glucan Peptide, CpG DNA, GM-CSF, GPI-0100, IFA, IFN-γ, IL-17, lipid A, lipopolysaccharide, Lipovant, Montanide, N-acetyl-muramyl-L-alanyl-D-isoglutamine, Pam3CSK4, quil A, trehalose dimycolate or zymosan.

In some embodiments, the adjuvant is an immunomodulatory molecule. For example, the immunomodulatory molecule may be a recombinant protein cytokine, chemokine, or immunostimulatory agent or nucleic acid encoding cytokines, chemokines, or immunostimulatory agents designed to enhance the immunologic response.

Examples of immunomodulatory cytokines include interferons (e.g., IFNα, IFNβ and IFNγ), interleukins (e.g., IL-1, IL-2, IL-3, IL-4, IL-5, IL-6, IL-7, IL-8, IL-9, IL-10, IL-12, IL-17 and IL-20), tumor necrosis factors (e.g., TNFα and TNFβ), erythropoietin (EPO), FLT-3 ligand, gIp10, TCA-3, MCP-1, MIF, MIP-lalpha, MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In some embodiments, an immunomodulatory chemokine that binds to a chemokine receptor, i.e., a CXC, CC, C, or CX3C chemokine receptor, also may be included in the compositions provided here. Examples of chemokines include, but are not limited to, Mip1α, Mip-1β, Mip-3α (Lam), Mip-3β, Rantes, Hcc-1, Mpif-1, Mpif-2, Mcp-1, Mcp-2, Mcp-3, Mcp-4, Mcp-5, Eotaxin, Tarc, Elc, 1309, IL-8, Gcp-2 Gro-α, Gro-β, Gro-γ, Nap-2, Ena-78, Gcp-2, Ip-10, Mig, I-Tac, Sdf-1, and Bca-1 (Bic), as well as functional fragments of any of the foregoing.

In some embodiments, the composition comprises a nucleic acid encoding an SARS-CoV-2 immunogenic polypeptide described herein, such as a DNA molecule encoding a SARS-CoV-2 immunogenic peptide. In some embodiments the composition comprises an expression vector comprising an open reading frame encoding a SARS-CoV-2 immunogenic peptide.

When taken up by a cell (e.g., muscle cell, an antigen-presenting cell (APC) such as a dendritic cell, macrophage, etc.), a DNA molecule may be present in the cell as an extrachromosomal molecule and/or may integrate into the chromosome. DNA may be introduced into cells in the form of a plasmid which may remain as separate genetic material. Alternatively, linear DNAs that may integrate into the chromosome may be introduced into the cell. Optionally, when introducing DNA into a cell, reagents which promote DNA integration into chromosomes may be added.

### VI. Binding Moieties

In some aspects, a binding moiety that binds a peptide described herein and/or a stable MHC-peptide complex described herein are provided. For example, binding proteins like T cell receptors (TCRs), antibodies, and the like that specifically bind to the peptide and/or the stable MHC-peptide complex, such as with a K_{d} less than or equal to about 10⁻⁷ M (e.g., about 10⁻⁷, about 10⁻⁸, about 10⁻⁹, about 10⁻¹⁰, about 10⁻¹¹, about 10⁻¹², about 10⁻¹³, about 10⁻¹⁴), are provided.

In some embodiments, the MHC molecule comprises an MHC alpha chain that is an HLA serotype selected from the group consisting of HLA-A*02, HLA-A*03, HLA-A*01, HLA-A*11, HLA-A*24, and/or HLA-B*07. In some embodiments, the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, and HLA-A*0274 allele. In a specific embodiment, the HLA allele is HLA-A*0201. In some embodiments, the binding proteins are genetically engineered, isolated, and/or purified.

In some embodiments, the binding proteins provided herein comprise a constant region that is chimeric, humanized, human, primate, or rodent (e.g., rat or mouse). For example, a human variable region may be chimerized with a murine constant region or a murine variable region may be humanized with a human constant region and/or human framework regions. In some embodiments, the constant regions may be mutated to modify functionality (e.g., introduction of non-naturally occurring cysteine substitutions in opposing residue locations in TCR alpha and beta chains to provide disulfide bonds useful for increasing affinity between the TCR alpha and beta chains). Similarly, mutations may be made in the transmembrane domain of the constant region to modify functionality (e.g., increase hydrophobicity by introducing a non-naturally occurring substitution of a residue with a hydrophobic amino acid).

In some embodiments, each CDR of the binding protein has up to five amino acid substitutions, insertions, deletions, or a combination thereof as compared to a reference CDR sequence.

In some embodiments, the binding proteins disclosed herein may comprise a T cell receptor (TCR), an antigen-binding fragment of a TCR, or a chimeric antigen receptor (CAR). In some embodiments, the binding protein disclosed herein may comprise two polypeptide chains, each of which comprises a variable region comprising a CDR3 of a TCR alpha chain and a CDR3 of a TCR beta chain, or a CDR1, CDR2, and CDR3 of both a TCR alpha chain and a TCR beta chain. In some embodiments, a binding protein comprises a single chain TCR (scTCR), which comprises both the TCR V_{α} and TCR V_{β} domains, but only a single TCR constant domain (C_{α} or C_{β}). The term “chimeric antigen receptor” (CAR) refers to a fusion protein that is engineered to contain two or more naturally-occurring amino acid sequences linked together in a way that does not occur naturally or does not occur naturally in a host cell, which fusion protein can function as a receptor when present on a surface of a cell. CARs encompassed by the present invention may include an extracellular portion comprising an antigen-binding domain (i.e., obtained or derived from an immunoglobulin or immunoglobulin-like molecule, such as an antibody or TCR, or an antigen binding domain derived or obtained from a killer immunoreceptor from an NK cell) linked to a transmembrane domain and one or more intracellular signaling domains (optionally containing co-stimulatory domain(s)) (see, e.g., Sadelain et al. (2013) Cancer Discov. 3:388, Harris and Kranz (2016) Trends Pharmacol. Sci. 37:220, and Stone et al. (2014) Cancer Immunol. Immunother. 63:1163).

In some embodiments, the binding proteins (e.g., the TCR, antigen-binding fragment of a TCR, or chimeric antigen receptor (CAR)) disclosed herein is chimeric (e.g., comprises amino acid residues or motifs from more than one donor or species), humanized (e.g., comprises residues from a non-human organism that are altered or substituted so as to reduce the risk of immunogenicity in a human), or human.

Methods for producing engineered binding proteins, such as TCRs, CARs, and antigen-binding fragments thereof, are well-known in the art (e.g., Bowerman et al. (2009) Mol. Immunol. 5:3000, U.S. Pat. Nos. 6,410,319, 7,446,191, U.S. Pat. Publ. No. 2010/065818; U.S. Pat. No. 8,822,647, PCT Publ. No. WO 2014/031687, U.S. Pat. No. 7,514,537, and Brentjens et al. (2007) Clin. Cancer Res. 73:5426).

In some embodiments, the binding protein described herein is a TCR, or antigen-binding fragment thereof, expressed on a cell surface, wherein the cell surface-expressed TCR is capable of more efficiently associating with a CD3 protein as compared to endogenous TCR A binding protein encompassed by the present invention, such as a TCR, when expressed on the surface of a cell like a T cell, may also have higher surface expression on the cell as compared to an endogenous binding protein, such as an endogenous TCR In some embodiments, provided herein is a CAR, wherein the binding domain of the CAR comprises an antigen-specific TCR binding domain (see, e.g., Walseng et al. (2017) Scientific Reports 7:10713).

Also provided are modified binding proteins (e.g., TCRs, antigen-binding fragments of TCRs, or CARs) that may be prepared according to well-known methods using a binding protein having one or more of the V_{α} and/or V_{β} sequences disclosed herein as starting material to engineer a modified binding protein that may have altered properties from the starting binding protein. A binding protein may be engineered by modifying one or more residues within one or both variable regions (i.e., V_{α} and/or V_{β}), for example within one or more CDR regions and/or within one or more framework regions. Additionally or alternatively, a binding protein may be engineered by modifying residues within the constant region(s).

Another type of variable region modification is to mutate amino acid residues within the V^{α} and/or V_{β} CDR1, CDR2 and/or CDR3 regions to thereby improve one or more binding properties (e.g., affinity) of the binding protein of interest. Site-directed mutagenesis or PCR-mediated mutagenesis may be performed to introduce the mutation(s) and the effect on protein binding, or other functional property of interest, may be evaluated in in vitro or in vivo assays as described herein and provided in the Examples. In some embodiments, conservative modifications (as discussed above) may be introduced. The mutations may be amino acid substitutions, additions or deletions. In some embodiments, the mutations are substitutions. Moreover, typically no more than one, two, three, four or five residues within a CDR region are modified.

In some embodiments, binding proteins (e.g., TCRs, antigen-binding fragments of TCRs, or CARs) described herein may possess one or more amino acid substitutions, deletions, or additions relative to a naturally occurring TCR In some embodiments, each CDR of the binding protein has up to five amino acid substitutions, insertions, deletions, or a combination thereof as compared to a reference CDR sequence. Conservative substitutions of amino acids are well-known and may occur naturally or may be introduced when the binding protein is recombinantly produced. Amino acid substitutions, deletions, and additions may be introduced into a protein using mutagenesis methods known in the art (see, e.g., Sambrook et al. (2001) Molecular Cloning: A Laboratory Manual, 3d ed., Cold Spring Harbor Laboratory Press, NY). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare immunogen polypeptide variants (see, e.g., Sambrook et al. supra).

A variety of criteria known to the ordinarily skilled artisan indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In some embodiments, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS™, Align, the BLAST algorithm, or other algorithms described herein and practiced in the art).

In any of the embodiments described herein, an encoded binding protein (e.g., TCR, antigen-binding fragment of a TCR, or CAR) may comprise a “signal peptide” (also known as a leader sequence, leader peptide, or transit peptide). Signal peptides target newly synthesized polypeptides to their appropriate location inside or outside the cell. A signal peptide may be removed from the polypeptide during or once localization or secretion is completed. Polypeptides that have a signal peptide are referred to herein as a “pre-protein” and polypeptides having their signal peptide removed are referred to herein as “mature” proteins or polypeptides. In some embodiments, a binding protein (e.g., TCR, antigen-binding fragment of a TCR, or CAR) described herein comprises a mature V_{α} domain, a mature V_{β} domain, or both. In some embodiments, a binding protein (e.g., TCR, antigen-binding fragment of a TCR, or CAR) described herein comprises a mature TCR β-chain, a mature TCR α-chain, or both.

In some embodiments, the binding proteins are fusion proteins comprising: (a) an extracellular component comprising a TCR or antigen-binding fragment thereof; (b) an intracellular component comprising an effector domain or a functional portion thereof; and (c) a transmembrane domain connecting the extracellular and intracellular components. In some embodiments, the fusion protein is capable of specifically binding to a MHC-peptide antigen complex comprising a peptide epitope described herein in the context of an MHC molecule (e.g., a MHC class I molecule).

As used herein, an “effector domain” or “immune effector domain” is an intracellular portion or domain of a fusion protein or receptor that can directly or indirectly promote an immune response in a cell when receiving an appropriate signal. In some embodiments, an effector domain is from an immune cell protein or portion thereof or immune cell protein complex that receives a signal when bound (e.g., CD3C), or when the immune cell protein or portion thereof or immune cell protein complex binds directly to a target molecule and triggers signal transduction from the effector domain in an immune cell.

An effector domain may directly promote a cellular response when it contains one or more signaling domains or motifs, such as an intracellular tyrosine-based activation motif (ITAM), such as those found in costimulatory molecules. Without wishing to be bound by theory, it is believed that ITAMs are useful for T cell activation following ligand engagement by a T cell receptor or by a fusion protein comprising a T cell effector domain. In some embodiments, the intracellular component or functional portion thereof comprises an ITAM. Exemplary immune effector domains include but are not limited to those from, CD3ε, CD3δ, CD3ζ, CD25, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof. In some embodiments, an effector domain comprises a lymphocyte receptor signaling domain (e.g., CD3ζ or a functional portion or variant thereof).

In further embodiments, the intracellular component of the fusion protein comprises a costimulatory domain or a functional portion thereof selected from CD27, CD28, 4-1BB (CD137), OX40 (CD134), CD2, CDS, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that specifically binds with CD83, or a functional variant thereof, or any combination thereof. In some embodiments, the intracellular component comprises a CD28 costimulatory domain or a functional portion or variant thereof (which may optionally include a LL-GG mutation at positions 186-187 of the native CD28 protein (e.g., Nguyen et al. (2003) Blood 702:4320), a 4-1BB costimulatory domain or a functional portion or variant thereof, or both.

In some embodiments, an effector domain comprises a CD3E endodomain or a functional (e.g., signaling) portion thereof, or a functional variant thereof. In further embodiments, an effector domain comprises a CD27 endodomain or a functional (e.g., signaling) portion thereof, or a functional variant thereof. In further embodiments, an effector domain comprises a CD28 endodomain or a functional (e.g., signaling) portion thereof, or a functional variant thereof. In still further embodiments, an effector domain comprises a 4-1BB endodomain or a functional (e.g., signaling) portion thereof, or a functional variant thereof. In further embodiments, an effector domain comprises an OX40 endodomain or a functional (e.g., signaling) portion thereof, or a functional variant thereof. In further embodiments, an effector domain comprises a CD2 endodomain or a functional (e.g., signaling) portion thereof, or a functional variant thereof. In further embodiments, an effector domain comprises a CD5 endodomain or a functional (e.g., signaling) portion thereof, or a functional variant thereof. In further embodiments, an effector domain comprises an ICAM-1 endodomain or a functional (e.g., signaling) portion thereof, or a functional variant thereof. In further embodiments, an effector domain comprises a LFA-1 endodomain or a functional (e.g., signaling) portion thereof, or a functional variant thereof. In further embodiments, an effector domain comprises an ICOS endodomain or a functional (e.g., signaling) portion thereof, or a functional variant thereof.

An extracellular component and an intracellular component encompassed by the present invention are connected by a transmembrane domain. A “transmembrane domain,” as used herein, is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In some embodiments, the transmembrane domain comprises or is derived from a known transmembrane protein (e.g., a CD4 transmembrane domain, a CD8 transmembrane domain, a CD27 transmembrane domain, a CD28 transmembrane domain, or any combination thereof).

In some embodiments, the extracellular component of the fusion protein further comprises a linker disposed between the binding domain and the transmembrane domain. As used herein when referring to a component of a fusion protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker encompassed by the present invention can position the binding domain away from the surface of a host cell expressing the fusion protein to enable proper contact between the host cell and a target cell, antigen binding, and activation (Patel et al. (1999) Gene Therapy 6:412-419). Linker length may be varied to maximize antigen recognition based on the selected target molecule, selected binding epitope, or antigen binding domain seize and affinity (see, e.g., Guest et al. (2005) Immunother. 28:203-11 and PCT Publ. No. WO 2014/031687). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂, (Gly₃Ser)₂, Gly₂Ser, or a combination thereof, such as ((Gly₃Ser)₂Gly₂Ser)).

In some embodiments, binding moieties encompassed by the present invention may be engineered protein scaffolds, an antibody or an antigen-binding fragment thereof, TCR-mimic antibodies, and the like. Such binding moieties may be designed and/or generated against peptides and/or MHC-peptide complexes described herein using routine immunological methods, such as immunizing a host, obtaining antibody-producing cells and/or antibodies thereof, and generating hybridomas useful for producing monoclonal antibodies (e.g., Watt et al. (2006) Nat. Biotechnol. 24:177-183; Gebauer and Skerra (2009) Curr. Opin. Chem Biol. 13:245-255; Skerra et al. (2008) FEBS J. 275:2677-2683; Nygren et al. (2008) FEBS J. 275:2668-2676; Dana et al. (2012) Exp. Rev. Mol. Med. 14:e6; Sergeva et al. (2011) Blood 117:4262-4272; PCT Publ. Nos. WO 2007/143104, PCT/US86/02269, and WO 86/01533; U.S. Pat. No. 4,816,567; Better et al. (1988) Science 240:1041-1043; Liu et al. (1987) Proc. Natl. Acad. Sci. U.S.A. 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. If desired, binding moieties may be isolated or purified using conventional procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography, lectin chromatography, and high performance liquid chromatography (HPLC) (e.g., Current Protocols in Immunology, or Current Protocols in Protein Science, John Wiley & Sons, NY, N.Y.).

The terms “antibody” and “antibodies” broadly encompass naturally-occurring forms of antibodies (e.g. IgG, IgA, IgM, IgE) and recombinant antibodies, such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In addition, intrabodies are well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publ. Nos. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a peptide and/or an MHC-peptide complex described herein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, protein subunit peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. Preferably, antibodies encompassed by the present bind specifically or substantially specifically to a peptide and/or an MHC-peptide complex described herein. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Similar to other binding moieties described herein, antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies encompassed by the present may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, have been grafted onto human framework sequences.

Binding proteins encompassed by the present invention may, in some embodiments, be covalently linked to a moiety. In some embodiments, the covalently linked moiety comprises an affinity tag or a label. The affinity tag may be selected from the group consisting of Glutathione-S-Transferase (GST), calmodulin binding protein (CBP), protein C tag, Myc tag, HaloTag, HA tag, Flag tag, His tag, biotin tag, and V5 tag. The label may be a fluorescent protein. In some embodiments, the covalently linked moiety is selected from the group consisting of an inflammatory agent, an anti-inflammatory agent, a cytokine, a toxin, a cytotoxic molecule, a radioactive isotope, or an antibody such as a single-chain Fv.

A binding protein may be conjugated to an agent used in imaging, research, therapeutics, theranostics, pharmaceuticals, chemotherapy, chelation therapy, targeted drug delivery, and radiotherapy. In some embodiments, a binding protein may be conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, 10, or more detectable moieties may be linked to a binding protein. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent Non-limiting examples of fluorescent dyes that may be used as a conjugating molecule include DyLight-680, DyLight-750, VivoTag-750, DyLight-800, IRDye-800, VivoTag-680, Cy5.5, ZQ800, or indocyanine green (ICG). In some embodiments, near infrared dyes often include cyanine dyes (e.g., Cy7, Cy5.5, and Cy5). Additional, non-limiting examples of fluorescent dyes for use as a conjugating molecule in accordance with present invention include acradine orange or yellow, Alexa Fluors® (e.g., Alexa Fluor® 790, 750, 700, 680, 660, and 647) and any derivative thereof, 7-actinomycin D, 8-anilinonaphthalene-1-sulfonic acid, ATTO® dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight® Fluors® and any derivative thereof, epicocconone, ethidium bromide, FlAsH-EDT2®, Fluo dye and any derivative thereof, FluoProbe® and any derivative thereof, fluorescein and any derivative thereof, Fura® and any derivative thereof, GelGreen® and any derivative thereof, GelRed® and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluorescent protein and YOYO-1. Other suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green™ dyes (e.g., Oregon Green™ 488, 500, 514, etc.), Texas Red®, Texas Red®-X, SPECTRUM RED®, SPECTRUM GREEN®, cyanine dyes (e.g., CY-3, Cy-5, CY-3.5, CY-5.5, etc.), Alexa Fluor® dyes (e.g., Alexa Fluor® 350, 488, 532, 546, 568, 594, 633, 660, 680, etc.), BODIPY® dyes (e.g., BODIPY® FL, R6G, TMR, TR, 530/550, 558/568, 564/570, 576/589, 581/591, 630/650, 650/665, etc.), IRD dyes (e.g., IRD40™, IRD700™, IRD800™, etc.), and the like. Additional suitable detectable agents are well-known in the art (e.g., PCT Publ. No. PCT/US14/56177). Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Binding proteins may be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cells of interest (e.g., immune cells) using both a therapeutic agent (e.g., drug) and electromagnetic energy (e.g., radiation or light) concurrently. In some embodiments, the binding protein is fused with, or covalently or non-covalently linked to the agent, for example, directly or via a linker.

In some embodiments, the binding protein may be chemically modified. For example, a binding protein may be mutated to modify peptide properties such as detectability, stability, biodistribution, pharmacokinetics, half-life, surface charge, hydrophobicity, conjugation sites, pH, function, and the like. N-methylation is one example of methylation that can occur in a binding protein encompassed by the present invention. In some embodiments, a binding protein may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a binding protein with an Fc region may be a fusion Fc-protein. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing.

In some embodiments, the binding proteins encompassed by the present invention may be modified. In some embodiments, the modifications having substantial or significant sequence identity to a parent binding protein to generate a functional variant that maintains one or more biophysical and/or biological activities of the parent binding protein (e.g., maintain binding specificity). In some embodiments, the mutation is a conservative amino acid substitution.

In some embodiments, binding proteins encompassed by the present invention may comprise synthetic amino acids in place of one or more naturally-occurring amino acids. Such synthetic amino acids are well-known in the art, and include, for example, aminocyclohexane carboxylic acid, norleucine, a-amino n-decanoic acid, homoserine, 5-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, a-aminocyclopentane carboxylic acid, oc-aminocyclohexane carboxylic acid, a-aminocycloheptane carboxylic acid, a-(2-amino-2-norbornane)-carboxylic acid, α,γ-diaminobutyric acid, β-diaminopropionic acid, homophenylalanine, and oc-tert-butylglycine.

Binding proteins encompassed by the present invention may be modified, such as glycosylated, amidated, carboxylated, phosphorylated, esterified, N-acylated, cyclized (e.g., via a disulfide bridge), or converted into an acid addition salt and/or optionally dimerized or polymerized, or conjugated.

In some embodiments, the attachment of a hydrophobic moiety, such as to the N-terminus, the C-terminus, or an internal amino acid, may be used to extend half-life of a peptide encompassed by the present invention. In other embodiments, a binding protein may include post-translational modifications (e.g., methylation and/or amidation), which can affect, for example, serum half-life. In some embodiments, simple carbon chains (e.g., by myristoylation and/or palmitylation) may be conjugated to the binding proteins. In some embodiments, the simple carbon chains may render the binding proteins easily separable from the unconjugated material. For example, methods that may be used to separate the binding proteins from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the binding proteins may be conjugated to myristic acid (tetradecanoic acid) or a derivative thereof. In other embodiments, a binding protein may be coupled (e.g., conjugated) to a half-life modifying agent Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a binding protein, such as 1, 2, 3, 4, 5, 6, 7, 8, 9, 10, or more amino acid residues that serve as a spacer or linker in order to facilitate conjugation or fusion to another molecule, as well as to facilitate cleavage of the peptide from such conjugated or fused molecules. In some embodiments, binding proteins may be conjugated to other moieties that, for example, can modify or effect changes to the properties of the binding proteins.

A binding protein may be produced recombinantly or synthetically, such as by solid-phase peptide synthesis or solution-phase peptide synthesis. Polypeptide synthesis may be performed by known synthetic methods, such as using fluorenylmethyloxycarbonyl (Fmoc) chemistry or by butyloxycarbonyl (Boc) chemistry. Polypeptide fragments may be joined together enzymatically or synthetically.

In an aspect encompassed by the present invention, provided herein are methods of producing a binding protein described herein, comprising the steps of: (i) culturing a transformed host cell which has been transformed by a nucleic acid comprising a sequence encoding a binding protein described herein under conditions suitable to allow expression of said binding protein; and (ii) recovering the expressed binding protein.

Methods useful for isolating and purifying recombinantly produced binding protein, by way of example, may include obtaining supernatants from suitable host cell/vector systems that secrete the binding protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of binding proteins described herein include batch cell culture, which is monitored and controlled to maintain appropriate culture conditions. Purification of the binding protein may be performed according to methods described herein and known in the art.

A variety of assays are well-known for assessing binding affinity and/or determining whether a binding molecule specifically binds to a particular ligand (e.g., peptide antigen-MHC complex). It is within the level of a skilled artisan to determine the binding affinity of a binding protein for a target, such as a T cell peptide epitope of a target polypeptide, such as by using any of a number of binding assays that are well-known in the art. For example, in some embodiments, a Biacore™ machine may be used to determine the binding constant of a complex between two proteins. The dissociation constant (K_{D}) for the complex may be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore™) analysis (see, e.g., Scatchard et al. (1949) Ann. N.Y. Acad. Sci. 51:660, Wilson (2002) Science 295:2103, Wolff et al. (1993) Cancer Res. 53:2560, and U.S. Pat. Nos. 5,283,173 and 5,468,614), flow cytometry, sequencing and other methods for detection of expressed nucleic acids. In one example, apparent affinity for a target is measured by assessing binding to various concentrations of tetramers, for example, by flow cytometry using labeled multimers, such as MHC-antigen peptide tetramers. In one representative example, apparent K_{D} of a binding protein is measured using 2-fold dilutions of labeled tetramers at a range of concentrations, followed by determination of binding curves by non-linear regression, apparent K_{D} being determined as the concentration of ligand that yielded half-maximal binding.

### VII. Uses and Methods

a. Diagnostic Methods

In some aspects, provided herein are diagnostic methods for determining whether a subject has exposure to and/or protection from SARS-CoV-2 comprising: (a) incubating a sample (e.g., blood, isolated PBMCs or isolated T cells) obtained from the subject with a SARS-CoV-2 immunogenic peptides described herein (e.g., a peptide epitope selected from Table 1A, 1B, 1C, 1D, 1E, and/or 1F), a MHC-peptide complex described herein, or a cell encoding and/or presenting a MHC-peptide complex described herein, such as from an immunogenic polypeptide construct described herein; and (b) detecting the level of reactivity; wherein a higher level of reactivity compared to a control level indicates that the subject has exposure to and/or protection from SARS-CoV-2.

In some embodiments, the level of reactivity is indicated by T cell activation or effector function, such as, but not limited to, T cell proliferation, killing, or cytokine release. The control level may be a reference number or a level of a healthy subject who has no exposure to SARS-CoV-2.

b. Therapeutic Methods

In some aspects, provided herein are methods for preventing and/or treating COVID-19 (i.e., a SARS-CoV-2 infection), and/or for inducing an immune response against a SARS-CoV-2 protein or fragment thereof. In certain embodiments, the method comprises administering to a subject an immunogenic composition described herein.

The methods described herein may be used to treat any subject in need thereof. As used herein, a “subject in need thereof” includes any subject who has COVID-19, who has had COVID-19 and/or who is predisposed to COVID-19. For example, in some embodiments, the subject has a COVID-19. In some embodiments, the subject has undergone treatments for COVID-19. In some embodiments, the subject is predisposed to COVID-19 due to age, or having a compromised immune system or other serious underlying medical conditions that predisposes the subject to COVID-19.

The pharmaceutical compositions disclosed herein may be delivered by any suitable route of administration, including orally and parenterally. In certain embodiments the pharmaceutical compositions are delivered generally (e.g., via oral or parenteral administration). In specific embodiments, the pharmaceutical compositions is administered by subcutaneous injection.

The dosage of the subject agent may be determined by reference to the plasma concentrations of the agent. For example, the maximum plasma concentration (Cmax) and the area under the plasma concentration-time curve from time 0 to infinity (AUC (0-4)) may be used. Dosages include those that produce the above values for Cmax and AUC (0-4) and other dosages resulting in larger or smaller values for those parameters.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the agents employed in the pharmaceutical composition at levels lower than that required in order to achieve the desired therapeutic effect and gradually increase the dosage until the desired effect is achieved.

In general, a suitable daily dose of an agent described herein will be that amount of the agent which is the lowest dose effective to produce a therapeutic effect. Such an effective dose will generally depend upon the factors described above.

In some embodiments, the immunogenic composition comprises an amount of a SRS-CoV-2 immunogenic peptide in combination with an adjuvant that constitutes a pharmaceutical dosage unit. A pharmaceutical dosage unit is defined herein as the amount of active ingredients (e.g., SRS-CoV-2 immunogenic peptides and/or adjuvant) that is applied to a subject at a given time point. A pharmaceutical dosage unit may be applied to a subject in a single volume, e.g., a single shot, or may be applied in 2, 3, 4, 5 or more separate volumes or shots that are applied at different locations of the body, for instance in the right and the left limb. Reasons for applying a single pharmaceutical dosage unit in separate volumes may be multiples, such as avoid negative side effects, avoiding antigenic competition and/or composition analytics considerations. It is to be understood herein that the separate volumes of a pharmaceutical dosage may differ in composition, i.e., may comprise different kinds or composition of active ingredients and/or adjuvants.

A pharmaceutical dosage unit may be an effective amount or part of an effective amount. An “effective amount” is to be understood herein as an amount or dose of active ingredients required to prevent and/or reduce the symptoms of a disease (e.g., COVID-19) relative to an untreated patient. The effective amount of active compound(s) used to practice the present invention for preventive and/or therapeutic treatment of COVID-19 varies depending upon the manner of administration, the age, body weight, and general health of the subject. Ultimately, the attending physician or veterinarian will decide the appropriate amount and dosage regimen. Such amount is referred to as an “effective” amount. This effective amount may also be the amount that is able to induce an effective cellular T cell response in the subject to be treated, or more preferably an effective systemic cellular T cell response.

In one aspect, provided herein is a method of eliciting in a subject an immune response to a cell that is infected with SARS-CoV-2 virus. The method comprises: administering to the subject a pharmaceutical composition described herein, wherein the pharmaceutical composition, when administered to the subject, elicits an immune response to the cell that is infected with SARS-CoV-2 virus.

Generally, the immune response may include a humoral immune response, a cell-mediated immune response, or both.

A humoral response may be determined by a standard immunoassay for antibody levels in a serum sample from the subject receiving the pharmaceutical composition. A cellular immune response is a response that involves T cells and may be determined in vitro or in vivo. For example, a general cellular immune response may be determined as the T cell proliferative activity in cells (e.g., peripheral blood leukocytes (PBLs)) sampled from the subject at a suitable time following the administering of a pharmaceutical composition. Following incubation of e.g., PBMCs with a stimulator for an appropriate period, [³H]thymidine incorporation may be determined. The subset of T cells that is proliferating may be determined using flow cytometry.

In certain aspects, the methods provided herein include administering to both human and non-human mammals. Veterinary applications also are contemplated. In some embodiments, the subject may be any living organism in which an immune response may be elicited. Examples of subjects include, without limitation, humans, livestock, dogs, cats, mice, rats, and transgenic species thereof.

In some embodiments, the pharmaceutical composition may be administered at any time that is appropriate. For example, the administering may be conducted before or during treatment of a subject having a COVID-19, and continued after the SARS-CoV-2 infection becomes clinically undetectable. The administering also may be continued in a subject showing signs of recurrence.

In some embodiments, the pharmaceutical composition may be administered in a therapeutically or a prophylactically effective amount. Administering the pharmaceutical composition to the subject may be carried out using known procedures, and at dosages and for periods of time sufficient to achieve a desired effect.

In some embodiments, the pharmaceutical composition may be administered to the subject at any suitable site. The route of administering may be parenteral, intramuscular, subcutaneous, intradermal, intraperitoneal, intranasal, intravenous (including via an indwelling catheter), via an afferent lymph vessel, or by any other route suitable in view of the subject's condition. Preferably, the dose will be administered in an amount and for a period of time effective in bringing about a desired response, be it eliciting the immune response or the prophylactic or therapeutic treatment of the SARS-CoV-2 infection and/or symptoms associated therewith.

The pharmaceutical composition may be given subsequent to, preceding, or contemporaneously with other therapies including therapies that also elicit an immune response in the subject. For example, the subject may previously or concurrently be treated by other forms of immunomodulatory agents, such other therapies preferably provided in such a way so as not to interfere with the immunogenicity of the compositions described herein.

Administering may be properly timed by the care giver (e.g., physician, veterinarian), and may depend on the clinical condition of the subject, the objectives of administering, and/or other therapies also being contemplated or administered. In some embodiments, an initial dose may be administered, and the subject monitored for an immunological and/or clinical response. Suitable means of immunological monitoring include using patient's peripheral blood lymphocyte (PBL) as responders and immunogenic peptides or MHC-peptide complexes described herein as stimulators. An immunological reaction also may be determined by a delayed inflammatory response at the site of administering. One or more doses subsequent to the initial dose may be given as appropriate, typically on a monthly, semimonthly, or a weekly basis, until the desired effect is achieved. Thereafter, additional booster or maintenance doses may be given as required, particularly when the immunological or clinical benefit appears to subside.

c. Methods of Identifying Molecules that Bind to a Peptide in the Context of an MHC Molecule

In some aspect, provide herein are methods of identifying a peptide-binding molecule or antigen-binding fragment thereof that binds to a peptide epitope selected from Table 1A, 1B, 1C, 1D, 1E, and/or 1F.

In some embodiments, the peptide binding molecule, i.e., MHC-peptide binding molecule, is a molecule or portion thereof that possesses the ability to bind, e.g., specifically bind, to a peptide epitope that is presented or displayed in the context of an MHC molecule (MHC-peptide complex), such as on the surface of a cell. Exemplary peptide binding molecules include T cell receptors or antibodies, or antigen-binding portions thereof, including single chain immunoglobulin variable regions (e.g., scTCR, scFv) thereof, that exhibit specific ability to bind to an MHC-peptide complex. In some embodiments, the peptide binding molecule is a TCR or antigen-binding fragment thereof. In some embodiments, the peptide binding molecule is an antibody, such as a TCR-like antibody or antigen-binding fragment thereof. In some embodiments, the peptide binding molecule is a TCR-like CAR that contains an antibody or antigen binding fragment thereof, such as a TCR-like antibody, such as one that has been engineered to bind to MHC-peptide complexes. In some embodiments, the peptide binding molecule may be derived from natural sources, or it may be partly or wholly synthetically or recombinantly produced.

In some embodiments, a binding molecule that binds to a peptide epitope may be identified by contacting one or more candidate peptide binding molecules, such as one or more candidate TCR molecules, antibodies or antigen-binding fragments thereof, with an MHC-peptide complex, and assessing whether each of the one or more candidate binding molecules binds, such as specifically binds, to the MHC-peptide complex. The methods may be performed in vitro, ex vivo or in vivo. Methods are well-known in the art for screening, such as described in U.S. Pat. Publ. 2020/0102553.

In some embodiments, the methods include contacting a plurality or library of binding molecules, such as a plurality or library of TCRs or antibodies, with an MHC-restricted epitope and identifying or selecting molecules that specifically bind such an epitope. In some embodiments, a library or collection containing a plurality of different binding molecules, such as a plurality of different TCRs or a plurality of different antibodies, may be screened or assessed for binding to an MHC-restricted epitope. In some embodiments, such as for selecting an antibody molecule that specifically binds an MHC-restricted peptide, hybridoma methods may be employed.

In some embodiments, screening methods may be employed in which a plurality of candidate binding molecules, such as a library or collection of candidate binding molecules, are individually contacted with an peptide binding molecule, either simultaneously or sequentially. Library members that specifically bind to a particular MHC-peptide complex may be identified or selected. In some embodiments, the library or collection of candidate binding molecules may contain at least 2, 5, 10, 100, 10³, 10⁴, 10⁵, 10⁶, 10⁷, 10⁸, 10⁹, or more different peptide binding molecules.

In some embodiments, the methods may be employed to identify a peptide binding molecule, such as a TCR or an antibody, that exhibits binding for more than one MHC haplotype or more than one MHC allele. In some embodiments, the peptide binding molecule, such as a TCR or antibody, specifically binds or recognizes a peptide epitope presented in the context of a plurality of MHC class I haplotypes or alleles. In some embodiments, the peptide binding molecule, such as a TCR or antibody, specifically binds or recognizes a peptide epitope presented in the context of a plurality of MHC class II haplotypes or alleles.

A variety of assays are known for assessing binding affinity and/or determining whether a binding molecule specifically binds to a particular ligand (e.g., MHC-peptide complex). It is within the level of a skilled artisan to determine the binding affinity of a TCR for a T cell epitope of a target polypeptide, such as by using any of a number of binding assays that are well known in the art. For example, in some embodiments, a BIAcore machine may be used to determine the binding constant of a complex between two proteins. The dissociation constant (Ku) for the complex may be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore®) analysis (see, e.g., Scatchard et al. (1949) Ann. N.Y. Acad. Sci. 51:660; Wilson (2002) Science 295:2103; Wolff et al. (1993) Cancer Res. 53:2560; and U.S. Pat. Nos. 5,283,173, 5,468,614, or the equivalent), flow cytometry, sequencing and other methods for detection of expressed nucleic acids. In one example, apparent affinity for a TCR is measured by assessing binding to various concentrations of tetramers, for example, by flow cytometry using labeled tetramers. In one example, apparent K_{D} of a TCR is measured using 2-fold dilutions of labeled tetramers at a range of concentrations, followed by determination of binding curves by non-linear regression, apparent K_{D} being determined as the concentration of ligand that yielded half-maximal binding.

In some embodiments, the methods may be used to identify binding molecules that bind only if the particular peptide is present in the complex, and not if the particular peptide is absent or if another, non-overlapping or unrelated peptide is present. In some embodiments, the binding molecule does not substantially bind the MHC in the absence of the bound peptide, and/or does not substantially bind the peptide in the absence of the MHC. In some embodiments, the binding molecules are at least partially specific. In some embodiments, an exemplary identified binding molecule may bind to an MHC-peptide complex if the particular peptide is present, and also bind if a related peptide that has one or two substitutions relative to the particular peptide is present.

In some embodiments, an identified antibody, such as a TCR-like antibody, may be used to produce or generate a chimeric antigen receptors (CARs) containing a non-TCR antibody that specifically binds to a MHC-peptide complex.

In some embodiments, the methods of identifying a peptide binding molecule, such as a TCR or TCR-like antibody or TCR-like CAR, may be used to engineer cells expressing or containing an peptide binding molecule. In some embodiments, a cell or engineered cell is a T cell. In some embodiments, the T cell is a CD4+ or CD8+ T cell. In some embodiments, the peptide binding molecule recognizes a MHC class I peptide complex, an MHC class II peptide complex and/or an MHC-E peptide complex. In some embodiments, an peptide binding molecule, such as a TCR or antibody or CAR, that specifically recognizes a peptide in the context of an MHC class I may be used to engineer CD8+ T cells. In some embodiments, also provided is a composition of engineered CD8+ T cells expressing or containing the TCR, antibody or CAR, for recognition of a peptide presented in the context of MHC class I. In any of such embodiments, the cells may be used in methods of adoptive cell therapy.

In some embodiments, TCR libraries may be generated by amplification of the repertoire of Vα, and Vβ from T cells isolated from a subject, including cells present in PBMCs, spleen or other lymphoid organ. In some cases, T cells may be amplified from tumor-infiltrating lymphocytes (TILs). In some embodiments, TCR libraries may be generated from CD4+ or CD8+ cells. In some embodiments, the TCRs may be amplified from a T cell source of a normal of healthy subject, i.e., normal TCR libraries. In some embodiments, the TCRs may be amplified from a T cell source of a diseased subject, i.e., diseased TCR libraries. In some embodiments, degenerate primers are used to amplify the gene repertoire of Vα, and Vβ, such as by RT-PCR in samples, such as T cells, obtained from humans. In some embodiments, scTv libraries may be assembled from naive Vα and Vβ libraries in which the amplified products are cloned or assembled to be separated by a linker. Depending on the source of the subject and cells, the libraries may be HLA allele-specific.

Alternatively, in some embodiments, TCR libraries may be generated by mutagenesis or diversification of a parent or scaffold TCR molecule. For example, in some aspects, a subject, e.g., human or other mammal such as a rodent, may be vaccinated with a peptide, such as a peptide identified by the present methods. In some embodiments, a sample may be obtained from the subject, such as a sample containing blood lymphocytes. In some instances, binding molecules, e.g., TCRs, may be amplified out of the sample, e.g., T cells contained in the sample. In some embodiments, antigen-specific T cells may be selected, such as by screening to assess CTL activity against the peptide. In some aspects, TCRs, e.g., present on the antigen-specific T cells, may be selected, such as by binding activity, e.g., particular affinity or avidity for the antigen. In some aspects, the TCRs are subjected to directed evolution, such as by mutagenesis, e.g., of the α or β chain. In some aspects, particular residues within CDRs of the TCR are altered. In some embodiments, selected TCRs may be modified by affinity maturation. In some aspects, a selected TCR may be used as a parent scaffold TCR against the antigen.

In some embodiments, the subject is a human, such as a human with COVID-19. In some embodiments, the subject is a rodent, such as a mouse. In some such embodiments, the mouse is a transgenic mouse, such as a mouse expressing human MHC (i.e., HLA) molecules, such as HLA-A2. See Nicholson et al. Adv Hematol. 2012; 2012: 404081.

In some embodiments, the subject is a transgenic mouse expressing human TCRs or is an antigen-negative mouse. See Li et al. (2010) Nat Med. 161029-1034; Obenaus et al. (2015) Nat Biotechnol. 33:402-407. In some aspects the subject is a transgenic mouse expressing human HLA molecules and human TCRs.

In some embodiments, such as where the subject is a transgenic HLA mouse, the identified TCRs are modified, e.g., to be chimeric or humanized. In some aspects, the TCR scaffold is modified, such as analogous to known antibody humanizing methods.

In some embodiments, such a scaffold molecule is used to generate a library of TCRs.

For example, in some embodiments, the library includes TCRs or antigen-binding portions thereof that have been modified or engineered compared to the parent or scaffold TCR molecule. In some embodiments, directed evolution methods may be used to generate TCRs with altered properties, such as with higher affinity for a specific MHC-peptide complex. In some embodiments, display approaches involve engineering, or modifying, a known, parent or reference TCR. For example, in some cases, a wild-type TCR may be used as a template for producing mutagenized TCRs in which in one or more residues of the CDRs are mutated, and mutants with an desired altered property, such as higher affinity for a desired target antigen, are selected. In some embodiments, directed evolution is achieved by display methods including, but not limited to, yeast display (Holler et al. (2003) Nat Immunol 4:55-62; Holler et al. (2000) Proc Natl Acad Sci USA 97:5387-5392), phage display (Li et al. (2005) Nat Biotechnol 23:349-354), or T cell display (Chervin et al. (2008) J Immunol Methods 339:175-184).

In some embodiments, the libraries may be soluble. In some embodiments, the libraries are display libraries in which the TCR is displayed on the surface of a phage or cell, or attached to a particle or molecule, such as a cell, ribosome or nucleic acid, e.g., RNA or DNA. Typically, the TCR libraries, including normal and disease TCR libraries or diversified libraries, may be generated in any form, including as a heterodimer or as a single chain form. In some embodiments, one or more members of the TCR may be a two-chain heterodimer. In some embodiments, pairing of the Vα and Vβ chains may be promoted by introduction of a disulfide bond. In some embodiments, members of the TCR library may be a TCR single chain (scTv or ScTCR), which, in some cases, may include a Vα and Vβ chain separated by a linker. Further, in some cases, upon screening and selection of a TCR from the library, the selected member may be generated in any form, such as a full-length TCR heterodimer or single-chain form or as antigen-binding fragments thereof.

Other methods of identifying molecules that bind to a peptide in the context of an MHC molecule are also described in U.S. Patent Application 2020/0182884, which is incorporated by reference herein in its entirety.

d. Monitoring of Effects During Clinical Trials

Monitoring the influence of a SARS-CoV-2 therapy (e.g., compounds, drugs, vaccines, or cell therapies) on T cell reactivity (e.g., the presence of binding and/or T cell activation and/or effector function), can be applied not only in basic candidate peptide-binding molecule screening, but also in clinical trials. For example, the effectiveness of SARS-CoV-2 immunogenic peptides or compositions, nucleic acids encoding such SARS-CoV-2 immunogenic peptides, MHC-peptide complexes, or cells expressing nucleic acids, vectors, immunogenic peptides or MHC-peptide complexes as described herein to increase immune response (e.g., T cell immune response) against SARS-CoV-2 infection, can be monitored in clinical trials of subjects afflicted with COVID-19. In such clinical trials, the presence of binding and/or T cell activation and/or effector function (e.g., T cell proliferation, killing, or cytokine release), can be used as a “read out” or marker of the phenotype of a particular cell, tissue, or system. Similarly, the effectiveness of an adaptive T cell therapy with T cells engineered to express a TCR determined by a screening assay as described herein, or with T cells that stimulated with immunogenic peptides, MHC-peptide complexes, or cells encoding and/or presenting MHC-peptide complexes as described herein to increase immune response to cells that are infected by SARS-CoV-2, can be monitored in clinical trials of subjects afflicted with COVID-19. In such clinical trials, the presence of binding and/or T cell activation and/or effector function (e.g., T cell proliferation, killing, or cytokine release), can be used as a “read out” or marker of the phenotype of a particular cell, tissue, or system.

In one embodiment, the present invention provides a method for monitoring the effectiveness of treatment of a subject with a SARS-CoV-2 therapy (e.g., compounds, drugs, vaccines, or cell therapies) including the steps of a) determining the presence or level of reactivity between T cells obtained from the subject and one or more immunogenic peptides or one or more stable MHC-peptide complexes described herein, in a first sample obtained from the subject prior to providing at least a portion of the SARS-CoV-2 therapy to the subject, and b) determining the presence or level of reactivity between the one more immunogenic peptides, or the one or more stable MHC-peptide complexes described herein, and T cells obtained from the subject present in a second sample obtained from the subject following provision of the portion of the SARS-CoV-2 therapy, wherein the presence or a higher level of reactivity in the second sample, relative to the first sample, is an indication that the therapy is efficacious for treating SARS-CoV-2 in the subject.

For example, increased administration of the SARS-CoV-2 therapy may be desirable to increase the presence or level of reactivity between T cells obtained from the subject and one or more immunogenic peptides or one or more stable MHC-peptide complexes described herein, i.e., to increase the effectiveness of the SARS-CoV-2 therapy. According to such an embodiment, the presence or level of reactivity between T cells obtained from the subject and one or more immunogenic peptides or one or more stable MHC-peptide complexes described herein may be used as an indicator of the effectiveness of a SARS-CoV-2 therapy, even in the absence of an observable phenotypic response. Similarly, analysis of the presence or level of reactivity between T cell and one or more immunogenic peptides or one or more stable MHC-peptide complexes described herein, such as by a direct binding assay, fluorescence activated cell sorting (FACS), enzyme linked immunosorbent assay (ELISA), radioimmune assay (RIA), immunochemically, Western blot, or intracellular flow assay, can also be used to select patients who will receive SARS-CoV-2 therapy.

For example, in a direct binding assay, immunogenic peptides or MHC-peptide complexes can be coupled with a radioisotope or enzymatic label such that binding can be determined by detecting the labeled immunogenic peptides or MHC-peptide complexes. For example, the immunogenic peptides or MHC-peptide complexes can be labeled with ¹²⁵I, ³⁵S, ¹⁴C, or ³H, either directly or indirectly, and the radioisotope detected by direct counting of radioemmission or by scintillation counting. Alternatively, the immunogenic peptides or MHC-peptide complexes can be enzymatically labeled with, for example, horseradish peroxidase, alkaline phosphatase, or luciferase, and the enzymatic label detected by determination of conversion of an appropriate substrate to product. Determining the interaction between immunogenic peptides or MHC-peptide complexes and T cells can also be accomplished using standard binding or enzymatic analysis assays. In one or more embodiments of the above described assay methods, it may be desirable to immobilize immunogenic peptides or MHC-peptide complexes to accommodate automation of the assay.

Binding of immunogenic peptides or MHC-peptide complexes to T cells can be accomplished in any vessel suitable for containing the reactants. Non-limiting examples of such vessels include microtiter plates, test tubes, and micro-centrifuge tubes. Immobilized forms of the immunogenic peptides or MHC-peptide complexes described herein can also include immunogenic peptides or MHC-peptide complexes bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In some embodiments, the reactivity of T cells to one or more immunogenic peptides or one or more stable MHC-peptide complexes described herein the presence of binding and/or T cell activation and/or effector function. The term “T cell activation” refers to T lymphocytes selected from proliferation, differentiation, cytokine secretion, release of cytotoxic effector molecules, cytotoxic activity, and expression of activation markers, particularly refers to one or more cellular responses of cytotoxic T lymphocytes.

The reactivity of T cells to one or more immunogenic peptides or one or more stable MHC-peptide complexes can be measured according to any of the T cell functional parameters described herein (e.g., proliferation, cytokine release, cytotoxicity, changes in cell surface marker phenotype, etc.).

Cytokine production and/or release can be measured by methods well known in the art, for example, ELISA, enzyme-linked immune absorbent spot (ELISPOT), Luminex® assay, intracellular cytokine staining, and flow cytometry, and combinations thereof (e.g., intracellular cytokine staining and flow cytometry). It can be determined according to the method implemented.

The term “cytokine” as used herein refers to a molecule that mediates and/or regulates a biological or cellular function or process (e.g., immunity, inflammation, and hematopoiesis). The term “cytokine” as used herein includes “lymphokines”, “chemokines”, “monokines”, and “interleukins”. Examples of useful cytokines are GM-CSF, IL-1α, IL-1β, IL-2, IL-3, IL-4, IL-5, IL-6, IL-7, IL-8, IL-10, IL-12, IL-15, IFN-α, IFN-β, IFN-γ, MIP-1α, MIP-1β, TGF-β, TNF-α, and TNF-β.

The proliferation and clonal expansion of T cells resulting from antigen-specific induction or stimulation of an immune response can be determined, for example, through incorporation of a non-radioactive assay such as a tritiated thymidine assay or MTT assay.

Cytotoxicity assays to determine CTL activity can be performed using any one of several techniques and methods routinely practiced in the art (e.g., Henkart et al. (2003) Fundamental Immunology 1127-1150). Additional description of methods for measuring antigen-specific T cell reactivity can be found in, for example, U.S. Pat. No. 10,208,086 and U.S. Patent Application 2017/0209573, each of which is incorporated by reference herein in its entirety.

### VIII. Cell Therapy

In certain aspects, the methods include adoptive cell therapy, whereby genetically engineered cells expressing the provided molecules targeting an MHC-restricted epitope (e.g., cells expressing a TCR or TCR-like CAR) are administered to subjects. Such administration may promote activation of the cells (e.g., T cell activation) in an antigen-targeted manner, such that the cells infected with SARS-CoV-2 are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition. In some aspects, the methods thereby treat, e.g., ameliorate one or more symptom of the disease or condition.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8:577-585). See, e.g., Themeli et al. (2013) Nat Biotechnot 31: 928-933; Tsukahara et al. (2013) Biochem Biophys Res Commun 438: 84-89; Davila et al. (2013) PLoS ONE 8:e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. In some embodiments, the primate is a monkey or an ape. The subject may be male or female and may be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS).

The binding molecules, such as TCRs, TCR-like antibodies and chimeric receptors (e.g., CARs) containing the TCR-like antibodies and cells expressing the same, may be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the binding molecule or cell may depend on the type of disease to be treated, the type of binding molecule, the severity and course of the disease, whether the binding molecule is administered for preventive or therapeutic purposes, previous therapy, the patient's clinical history and response to the binding molecule, and the discretion of the attending physician. The compositions and molecules and cells are in some embodiments suitably administered to the patient at one time or over a series of treatments.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about million cells, about 10 billion cells, about 25 billion cells, about 50 billion cells, about billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the cells or binding molecules (e.g., TCR or TCR-like antibodies) are administered as part of a combination treatment, such as simultaneously with or sequentially with, in any order, another therapeutic intervention, such as another antibody or engineered cell or receptor or agent, such as a cytotoxic or therapeutic agent.

The cells or binding molecules (e.g., TCR or TCR-like antibodies) in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells or binding molecules (e.g., TCR or TCR-like antibodies) are administered prior to the one or more additional therapeutic agents. In some embodiments, the cells or binding molecules (e.g., TCR or TCR-like antibodies) are administered after to the one or more additional therapeutic agents.

Once the cells are administered to a mammal (e.g., a human), the biological activity of the engineered cell populations and/or binding molecules (e.g., TCR or TCR-like antibodies) in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells may be measured using any suitable method known in the art, such as cytotoxicity assays described in, for example, Kochenderfer et al. (2009) J. Immunotherapy 32:689-702, and Herman et al. (2004) J. Immunological Methods 285:25-40. In certain embodiments, the biological activity of the cells also may be measured by assaying expression and/or secretion of certain cytokines, such as CD 107a, IFNγ, IL-2, and TNF. In some aspects the biological activity is measured by assessing clinical outcome, such as reduction in tumor burden or load.

In certain embodiments, engineered cells are modified in any number of ways, such that their therapeutic or prophylactic efficacy is increased. For example, the engineered CAR or TCR expressed by the population may be conjugated either directly or indirectly through a linker to a targeting moiety. The practice of conjugating compounds, e.g., the CAR or TCR, to targeting moieties is known in the art. See, for instance, Wadwa et al. (1995) J. Drug Targeting 3: 111, and U.S. Pat. No. 5,087,616.

In certain aspects, the SARS-CoV-2 immunogenic peptides described herein, or a nucleic acid encoding such SARS-CoV-2 immunogenic peptides, may be used in compositions and methods for providing SARS-CoV-2-primed, antigen-presenting cells, and/or SARS-CoV-2-specific lymphocytes generated with these antigen-presenting cells. In some embodiments, such antigen-presenting cells and/or lymphocytes are used in the treatment and/or prevention of COVID-19 (i.e., SARS-CoV-2 infection).

In some aspects, provided herein are methods for making SARS-CoV-2-primed, antigen-presenting cells by contacting antigen-presenting cells with a SARS-CoV-2 immunogenic polypeptide described herein, or nucleic acids encoding the at least one SARS-CoV-2 immunogenic polypeptide, alone or in combination with an adjuvant, in vitro under a condition sufficient for the at least one SARS-CoV-2 immunogenic polypeptide to be presented by the antigen-presenting cells.

In some embodiments, the SARS-CoV-2 immunogenic polypeptide, or nucleic acid encoding the SARS-CoV-2 immunogenic polypeptide, alone or in combination with an adjuvant, may be contacted with a homogenous, substantially homogenous, or heterogeneous composition comprising antigen-presenting cells. For example, the composition may include but is not limited to whole blood, fresh blood, or fractions thereof such as, but not limited to, peripheral blood mononuclear cells, buffy coat fractions of whole blood, packed red cells, irradiated blood, dendritic cells, monocytes, macrophages, neutrophils, lymphocytes, natural killer cells, and natural killer T cells. If, optionally, precursors of antigen-presenting cells are used, the precursors may be cultured under suitable culture conditions sufficient to differentiate the precursors into antigen-presenting cells. In some embodiments, the antigen-presenting cells (or precursors thereof) are selected from monocytes, macrophages, cells of myeloid lineage, B cells, dendritic cells, or Langerhans cells.

The amount of the SARS-CoV-2 immunogenic polypeptide, or nucleic acid encoding the SARS-CoV-2 immunogenic polypeptide, alone or in combination with an adjuvant, to be placed in contact with antigen-presenting cells may be determined by one of ordinary skill in the art by routine experimentation. Generally, antigen-presenting cells are contacted with the SARS-CoV-2 immunogenic polypeptide, or nucleic acid encoding the SARS-CoV-2 immunogenic polypeptide, alone or in combination with an adjuvant, for a period of time sufficient for cells to present the processed forms of the antigens for the modulation of T cells. In one embodiment, antigen-presenting cells are incubated in the presence of the SARS-CoV-2 immunogenic polypeptide, or nucleic acid encoding the SARS-CoV-2 immunogenic polypeptide, alone or in combination with an adjuvant, for less than about a week, illustratively, for about 1 minute to about 48 hours, about 2 minutes to about 36 hours, about 3 minutes to about 24 hours, about 4 minutes to about 12 hours, about 6 minutes to about 8 hours, about 8 minutes to about 6 hours, about 10 minutes to about 5 hours, about 15 minutes to about 4 hours, about 20 minutes to about 3 hours, about 30 minutes to about 2 hours, and about 40 minutes to about 1 hour. The time and amount of the SARS-CoV-2 immunogenic polypeptide, or nucleic acid encoding the SARS-CoV-2 immunogenic polypeptide, alone or in combination with an adjuvant, necessary for the antigen presenting cells to process and present the antigens may be determined, for example using pulse-chase methods wherein contact is followed by a washout period and exposure to a read-out system e.g., antigen reactive T cells.

In certain embodiments, any appropriate method for delivery of antigens to the endogenous processing pathway of the antigen-presenting cells may be used. Such methods include but are not limited to, methods involving pH-sensitive liposomes, coupling of antigens to adjuvants, apoptotic cell delivery, pulsing cells onto dendritic cells, delivering recombinant chimeric virus-like particles (VLPs) comprising antigen to the MHC class I processing pathway of a dendritic cell line.

In one embodiment, solubilized SARS-CoV-2 immunogenic polypeptide is incubated with antigen-presenting cells. In some embodiments, the SARS-CoV-2 immunogenic polypeptide may be coupled to a cytolysin to enhance the transfer of the antigens into the cytosol of an antigen-presenting cell for delivery to the MHC class I pathway. Exemplary cytolysins include saponin compounds such as saponin-containing Immune Stimulating Complexes (ISCOM5), pore-forming toxins (e.g., an alpha-toxin), and natural cytolysins of gram-positive bacteria such as listeriolysin O (LLO), streptolysin O (SLO), and perfringolysin O (PFO).

In some embodiments, antigen-presenting cells, such as dendritic cells and macrophage, may be isolated according to methods known in the art and transfected with polynucleotides by methods known in the art for introducing a nucleic acid encoding the SARS-CoV-2 immunogenic polypeptide into the antigen-presenting cell. Transfection reagents and methods are known in the art and commercially available. For example, RNA encoding SARS-CoV-2 immunogenic polypeptide may be provided in a suitable medium and combined with a lipid (e.g., a cationic lipid) prior to contact with antigen-presenting cells. Non-limiting examples of such lipids include LIPOFECTIN™ and LIPOFECTAMINE™. The resulting polynucleotide-lipid complex may then be contacted with antigen-presenting cells. Alternatively, the polynucleotide may be introduced into antigen-presenting cells using techniques such as electroporation or calcium phosphate transfection. The polynucleotide-loaded antigen-presenting cells may then be used to stimulate T lymphocyte (e.g., cytotoxic T lymphocyte) proliferation in vivo or ex vivo. In one embodiment, the ex vivo expanded T lymphocyte is administered to a subject in a method of adoptive immunotherapy.

In certain aspects, provided herein is a composition comprising antigen-presenting cells that have been contacted in vitro with a SARS-CoV-2 immunogenic polypeptide, or a nucleic acid encoding a SARS-CoV-2 immunogenic polypeptide, alone or in combination with an adjuvant under a condition sufficient for a SARS-CoV-2 immunogenic epitope to be presented by the antigen-presenting cells.

In some aspects, provided herein is a method for preparing lymphocytes specific for a SARS-CoV-2 protein. The method comprises contacting lymphocytes with the antigen-presenting cells described above under conditions sufficient to produce a SARS-CoV-2 protein-specific lymphocyte capable of eliciting an immune response against a cell that is infected by the SARS-CoV-2 virus. Thus, the antigen-presenting cells also may be used to provide lymphocytes, including T lymphocytes and B lymphocytes, for eliciting an immune response against cell that is infected by the SARS-CoV-2 virus.

In some embodiments, a preparation of T lymphocytes is contacted with the antigen-presenting cells described above for a period of time, (e.g., at least about 24 hours) to priming the T lymphocytes to a SARS-CoV-2 immunogenic epitope presented by the antigen-presenting cells.

In some embodiments, a population of antigen-presenting cells may be co-cultured with a heterogeneous population of peripheral blood T lymphocytes together with a SARS-CoV-2 immunogenic polypeptide, or a nucleic acid encoding a SARS-CoV-2 immunogenic polypeptide, alone or in combination with an adjuvant. The cells may be co-cultured for a period of time and under conditions sufficient for SARS-CoV-2 epitopes included in the SARS-CoV-2 polypeptides to be presented by the antigen-presenting cells and the antigen-presenting cells to prime a population of T lymphocytes to respond to cells is infected by the SARS-CoV-2 virus. In certain embodiments, provided herein are T lymphocytes and B lymphocytes that are primed to respond to cells that is infected by the SARS-CoV-2 virus.

T lymphocytes may be obtained from any suitable source such as peripheral blood, spleen, and lymph nodes. The T lymphocytes may be used as crude preparations or as partially purified or substantially purified preparations, which may be obtained by standard techniques including, but not limited to, methods involving immunomagnetic or flow cytometry techniques using antibodies.

In certain aspects, provided herein is a composition (e.g., a pharmaceutical composition) comprising the antigen-presenting cells or the lymphocytes described above, and a pharmaceutically acceptable carrier and/or diluent. In some embodiments, the composition further comprises an adjuvant as described above.

In certain aspects, provided herein is a method for eliciting an immune response to the cell is infected by the SARS-CoV-2 virus, the method comprising administering to the subject the antigen-presenting cells or the lymphocytes described above in effective amounts sufficient to elicit the immune response. In some embodiments, provided herein is a method for treatment or prophylaxis of COVID-19, the method comprising administering to the subject an effective amount of the antigen-presenting cells or the lymphocytes described above. In one embodiment, the antigen-presenting cells or the lymphocytes are administered systemically, preferably by injection. Alternately, one may administer locally rather than systemically, for example, via injection directly into tissue, preferably in a depot or sustained release formulation.

In certain embodiments, the antigen-primed antigen-presenting cells described herein and the antigen-specific T lymphocytes generated with these antigen-presenting cells may be used as active compounds in immunomodulating compositions for prophylactic or therapeutic treatment of COVID-19. In some embodiments, the SARS-CoV-2-primed antigen-presenting cells described herein may be used for generating CD8⁺ T lymphocytes, CD4⁺ T lymphocytes, and/or B lymphocytes for adoptive transfer to the subject. Thus, for example, SARS-CoV-2-specific lymphocyte may be adoptively transferred for therapeutic purposes in subjects afflicted with COVID-19.

In certain embodiments, the antigen-presenting cells and/or lymphocytes described herein may be administered to a subject, either by themselves or in combination, for eliciting an immune response, particularly for eliciting an immune response to cells are infected by the SARS-CoV-2 virus. In some embodiments, the antigen-presenting cells and/or lymphocytes may be derived from the subject (i.e., autologous cells) or from a different subject that is MHC matched or mismatched with the subject (e.g., allogeneic).

Single or multiple administrations of the antigen-presenting cells and lymphocytes may be carried out with cell numbers and treatment being selected by the care provider (e.g., physician). In some embodiments, the antigen-presenting cells and/or lymphocytes are administered in a pharmaceutically acceptable carrier. Suitable carriers may be growth medium in which the cells were grown, or any suitable buffering medium such as phosphate buffered saline. The cells may be administered alone or as an adjunct therapy in conjunction with other therapeutics.

### IX. Kits

The present invention also encompasses kits. For example, the kit may comprise immunogenic peptides, vectors comprising sequences encoding immunogenic peptides, stable MHC-peptide complexes as described herein, adjuvants, and combinations thereof, packaged in a suitable container and may further comprise instructions for using such reagents. The kit may also contain other components, such as administration tools packaged in a separate container.

The disclosure is further illustrated by the following examples which should not be construed as limiting. The contents of all references, patents and published patent applications cited throughout this application, as well as the Figures, are incorporated herein by reference.

### EXAMPLES

### Example 1: SARS-CoV-2 Vaccine Constructs

Studies were undertaken to construct a vaccine that would generate the strongest possible CD8 T cell response to SARS-CoV-2. This includes maximizing the magnitude of the response to each individual epitope, as well as the breadth of total targeted epitopes. At the same time, the vaccine design faces several constraints. One important consideration is the limited size of the overall vaccine construct; excessive size poses challenges for vaccine delivery systems (such as mRNA or viral vectors) and can impede the efficient expression of the full construct.

There is considerable skepticism in the field about the ability of polyepitope vaccines to generate really robust T cell responses. This skepticism centers on two main challenges. The first challenge concerns the limited set of epitopes included in a vaccine. A polyepitope vaccine mainly includes only the discrete encoded peptide epitopes. By contrast, a vaccine including an entire protein can be processed into thousands of different epitopes—with different epitopes presented on different MHC alleles. A protein the size of the SARS-CoV-2 S protein contains so many potential epitopes that there are numerous predicted high-affinity binders for every possible MHC allele. The challenge for a polyepitope vaccine is to elicit a stronger response against a limited, MHC-restricted, pre-defined set of epitopes than one would naturally get against entire proteins. The second challenge concerns the processing and presentation of the epitopes in the context of a polyepitope vaccine. Just because the epitopes discovered are efficiently presented in the context of a SARS-CoV-2 infection doesn't mean that they will be presented in the context of a polyepitope vaccine. This concern is particularly acute due to the limited number of epitopes included in a polyepitope vaccine; the failure to present even a modest fraction of the epitopes in the vaccine can have a dramatic effect on the ability of the vaccine to elicit a response at all on particular MHC alleles.

These challenges are broadly understood in the field and have hampered the development of successful polyepitope vaccines for other viruses. Indeed, even in light of recent SARS-CoV-2 epitope mapping work undertaken and identifying a limited set of highly immunodominant epitopes recognized on each MHC allele, considerable skepticism was faced around whether these biological insights could translate into an effective vaccine that overcomes these fundamental challenges.

There are two sets of open-ended decisions to be made in the design of a polyepitope vaccine, which include 1) the content included in the vaccine and 2) the context in which the content is expressed.

A broad range of options for both of these decisions were explored. A diverse set of vaccines were designed to examine experimentally, taking advantage of unique reagents previously generated (highly-active memory CD8 T cells from a group of convalescent COVID19 patients).

In particular, DNA sequences were backtranslated from the designed protein vaccine sequences and were then sequence optimized using the GenSmart™ codon optimization algorithm (GenScript, Inc.; see also PCT Publ. WO 2020/0024917). The DNA was ordered as gBlocks (Integrated DNA Technologies Inc.) and assembled into EcoRI-linearized pHAGE-CMV-IRES-Puro vector (Kula et al. (2019) Cell 178:1016-1028) using NEBuilder® HiFi DNA Assembly Master Mix (New England Biolabs; Cat. #E2621S). Assemblies were transformed into Mix & Go! chemically competent cells (Zymo Research; Cat. #T3007) and individual colonies were selected for Sanger sequencing.

A*02:01-expressing HEK293 cells were transduced with each vaccine construct at a multiplicity of infection (MOI) of 1 and selected using puromycin (1.5 ug/nit, Gibco). 5×10⁴ cells were seeded into 96 well plates and rested for 16 hr. Memory CD8 T cells isolated from convalescent COVID-19 patients (Ferretti et al. (2020) Immunity S1074-7613(20)30447-7; available at doi.org/10.1016/j.immuni.2020.10.006) were added at an effector to target ratio of 2:1 and incubated for 16-18 hours. Following incubation, the cells were mixed by pipetting, transferred to a V-bottom 96-well plate, and pelleted by centrifuging at 300×g for 5 minutes. The supernatant was collected and IFNgamma s measured using Ella (Protein Simple). The cell pellets were stained with B conjugated anti-CD8 (BioLegend), AF647-conjugated anti-CD69 (Biolegend), and PIE-conjugated anti-CD137 (Miltenyi) antibodies, and analyzed on a Cytoflex S (Beckman Coulter).

The results demonstrated that both the context and content of the vaccine has a profound impact on its performance in ways that were not predicted. Moreover, it was observed that the designed vaccine constructs hold great promise to generate more robust CD8 T cell responses than current vaccine candidates in clinical trials.

Immunodominant epitopes presented on the six most common MHC alleles were identified: HLA-A*02:01, HLA-A*01:01, HLA-A*03:01, HLA-A*11:01, HLA-A*24:02, and HLA-B*07:02. Polyepitope vaccines that encoded either all 29 immunodominant epitopes identified (6 A2, 8 A1, 4 A3, 5 A11, 3 A24, 3 B7) or 19 immunodominant epitopes (3-4 from each MHC) were designed. When selecting the 19 epitopes, epitopes that had predicted or validated presentation on multiple MHC alleles were included. These 19 epitopes include the subset from Table L1 identified below.

Table L below, composed of Table L1 and Table L2, identifies the 29 peptide epitopes that can be used in the disclosed constructs (e.g., sequences in Table L1, and additional data in Table L2). Note that Table L clearly demonstrates that the orf3a, M, and N, as well as orf1ab, proteins are hotspots for containing T cell epitopes.

TABLE L1

| | | | | | | | | Used in |
|---|---|---|---|---|---|---|---|---|
| | | | | | | | Multiple | 19 |
| | | Peptide | | | | | HLA | epitope |
| | Allele | Name | Peptide | Protein | Start | End | alleles | construct |
| 0 | A02 | KLW | KLWAQCVQL | ORF1ab | 3886 | 3894 | | Yes |
| 1 | A02 | YLQ | YLQPRTFLL | S | 269 | 277 | | Yes |
| 2 | A02 | LLY | LLYDANYFL | ORF3a | 139 | 147 | | Yes |
| 3 | A02 | ALW | ALWEIQQVV | ORF1ab | 4094 | 4102 | | Yes |
| 4 | A02 | LLL | LLLDRLNQL | N | 222 | 230 | | |
| 5 | A02 | YLF | YLFDESGEFKL | ORF1ab | 906 | 916 | | |
| 6 | A01 | FTS | FTSDYYQLY | ORF3a | 207 | 215 | | Yes |
| 7 | A01 | TTD | TTDPSFLGRY | ORF1ab | 1637 | 1646 | | Yes |
| 8 | A01 | PTD | PTDNYITTY | ORF1ab | 1321 | 1329 | | Yes |
| 9 | A01 | ATS | ATSRTLSYY | M | 171 | 179 | Yes | |
| | | | | | | | (A01 | |
| | | | | | | | and | |
| | | | | | | | A11) | |
| 10 | A01 | CTD | CTDDNALAYY | ORF1ab | 4163 | 4172 | | |
| 11 | A01 | NTC | NTCDGTTFTY | ORF1ab | 4082 | 4091 | | |
| 12 | A01 | DTD | DTDFVNEFY | ORF1ab | 5130 | 5138 | | |
| 13 | A01 | GTD | GTDLEGNFY | ORF1ab | 3437 | 3445 | | |
| 14 | A03 | KTF | KTFPPTEPK | N | 361 | 369 | Yes | Yes |
| | | | | | | | (A03 | |
| | | | | | | | and | |
| | | | | | | | A11) | |
| 15 | A03 | KCY | KCYGVSPTK | S | 378 | 386 | | Yes |
| 16 | A03 | MVT | MVTNNTFTLK | ORF1ab | 807 | 816 | | Yes |
| 17 | A03 | KTI | KTIQPRVEK | ORF1ab | 282 | 290 | | |
| 18 | A11 | KTF | KTFPPTEPK | N | 361 | 369 | Yes | |
| | | | | | | | (A03 | |
| | | | | | | | and | |
| | | | | | | | A11) | |
| 19 | A11 | VTD | VTDTPKGPK | ORF1ab | 4216 | 4224 | | Yes |
| 20 | A11 | ATE | ATEGALNTPK | N | 134 | 143 | | Yes |
| 21 | A11 | ASA | ASAFFGMSR | N | 311 | 319 | | |
| 22 | A11 | ATS | ATSRTLSYYK | M | 171 | 180 | Yes | Yes |
| | | | | | | | (A01 | |
| | | | | | | | and | |
| | | | | | | | A11) | |
| 23 | A24 | QYI | QYIKWPWYI | S | 1208 | 1216 | | Yes |
| 24 | A24 | VYF | VYFLQSINF | ORF3a | 112 | 120 | | Yes |
| 25 | A24 | VYI | VYIGDPAQL | ORF1ab | 5721 | 5729 | | Yes |
| 26 | B07 | SPR | SPRWYFYYL | N | 105 | 113 | | Yes |
| 27 | B07 | RPD | RPDTRYVL | ORF1ab | 2949 | 2956 | | Yes |
| 28 | B07 | IPR | IPRRNVATL | ORF1ab | 5916 | 5924 | | Yes |

TABLE L2

| | | Peptide | Affinity | % of Pts | % of Pts |
|---|---|---|---|---|---|
| | Allele | Name | (nM) | (Screen M+ 2SD) | (Tetramer) |
| 0 | A02 | KLW | 17.7 | 88.9 | 77.8 |
| 1 | A02 | YLQ | 5.4 | 77.8 | 44.4 |
| 2 | A02 | LLY | 3.1 | 88.9 | 55.6 |
| 3 | A02 | ALW | 7.8 | 88.9 | 25.9 |
| 4 | A02 | LLL | 14.8 | 33.3 | 22.2 |
| 5 | A02 | YLF | 22.2 | 44.4 | 18.5 |
| 6 | A01 | FTS | 3.2 | 100 | |
| 7 | A01 | TTD | 7.2 | 100 | |
| 8 | A01 | PTD | 6.1 | 80 | |
| 9 | A01 | ATS | 16.7 | 60 | |
| 10 | A01 | CTD | 5.3 | 100 | |
| 11 | A01 | NTC | 121.8 | 60 | |
| 12 | A01 | DTD | 2.8 | 40 | |
| 13 | A01 | GTD | 6 | 40 | |
| 14 | A03 | KTF | 20.8 | 100 | |
| 15 | A03 | KCY | 152.6 | 100 | |
| 16 | A03 | MVT | 19.8 | 40 | |
| 17 | A03 | KTI | 113.2 | 40 | |
| 18 | A11 | KTF | 6.3 | 100 | |
| 19 | A11 | VTD | 160.6 | 60 | |
| 20 | A11 | ATE | 55.5 | 80 | |
| 21 | A11 | ASA | 14.4 | 40 | |
| 22 | A1 | ATS | 7.9 | 60 | |
| 23 | A24 | QYI | 13.2 | 60 | |
| 24 | A24 | VYF | 47.4 | 80 | |
| 25 | A24 | VYI | 206 | 40 | |
| 26 | B07 | SPR | 6.3 | 80 | |
| 27 | B07 | RPD | 56.9 | 80 | |
| 28 | B07 | IPR | 5.1 | 20 | |

In terms of the vaccine context, one important factor is the linker between epitopes. The linker is a key determinant of whether the epitopes are likely to be efficiently processed and presented. Tested constructs had either no linker at all (direct concatenation), the 3aa upstream and downstream of each epitope (for a total of baa spacing between adjacent epitopes), or an optimized proteasomal cleavage sequence “KAA” between each set of epitopes. Additional work included also optimizing the order of the epitopes within the polyepitope vaccine. Here, the aim was to minimize the generation of junctional epitopes that bind MHC with high affinity but are not found in the SARS-CoV-2 genome; such epitopes would compete with the desired epitopes for presentation. An algorithm was developed to iteratively alter the order of the epitopes to remove the highest affinity predicted epitopes within a polyepitope construct. This algorithm was applied to 10,000 different starting constructs and the variant with the best final performance was selected. In particular, it was determined that the variable contributing a difference was the choice of which HLA alleles are considered when evaluating junctional neoepitopes. For the purpose of the algorithm, the 6 HLA alleles that the epitopes in Table L are presented on were selected. Next, a comprehensive set of all of the possible junctions between the epitopes selected were generated. The NetMHC4.0 algorithm (Jurtz et al. (2017) J. Immunol. 199:3360-3368) was used to determine the MHC binding epitopes on the 6 MHC alleles examined. For each junction, the highest-affinity predicted binder that was not derived from the natural viral sequence (i.e., arising due to the junction itself) was identified. Next, a random starting order for the epitopes was assigned. For the given order, the highest affinity predicted junctional epitope was identified. All of the possible ways in which two epitopes could be swapped within the construct were evaluated, and the epitope swap that resulted in the lowest affinity remaining junctional epitope was selected. This process was iterated until no epitope swap could further improve the construct. This entire algorithm was run with 10,000 random starting orders, and the best-performing final order was selected (i.e., the order in which the highest-affinity junctional epitope has the lowest possible affinity).

In the course of designing different constructs, the location of individual epitopes within the polyepitope vaccine were also varied. Finally, the designed polyepitope vaccines were constructed either as stand-alone constructs expressed in the cytoplasm, or in the context of polycistronic expression following a native SARS-CoV-2 S protein (using a P2A sequence to enable cytoplasmic expression of our polyepitope vaccine directly after the transmembrane expression of the S protein using its natural signal sequence).

To evaluate these constructs, lentiviral transduction (at a low MOI) was used to express each construct in HEK293T cells that expressed only HLA-A*02:01. The modified HEK293T cells were then co-cultured with the memory CD8 T cells from a panel of convalescent COVID-19 patients (e.g., A*02:01-positive COVID-19 patients). IFNgamma (IFNg) secretion was used to evaluate the strength of the T cell response against any presented epitopes. This provides a readout of how efficiently the epitopes are presented in the context of each vaccine. Based on prior work, it was known that most COVID19 convalescent patients generate memory CD8 T cells against the epitopes tested, so the strength of the response provided a direct readout of how efficiently the epitopes were efficiently presented in each vaccine construct.

Several important controls were included. First, HEK293T cells alone (without any viral sequence) were tested, which served as a negative control (“A2 Screps”). Second, peptide pulsing with a set of three immunodominant HLA-A*02:01 SARS-CoV-2 epitopes was used as a positive control (since peptide pulsing directly loads onto MHC with high efficiency) (“A2 Screps+KLW/YLQ/LLY”). Third, S protein alone was used. This is the lead construct that is currently besting tested in clinical trials, which allowed for a comparison of the magnitude of the elicited T cell response by the polyepitope constructs to this baseline. Finally, large fragments of ˜500 aa spanning each of the three immunodominant HLA-A*02:01 epitopes were also included in the pulse experiments.

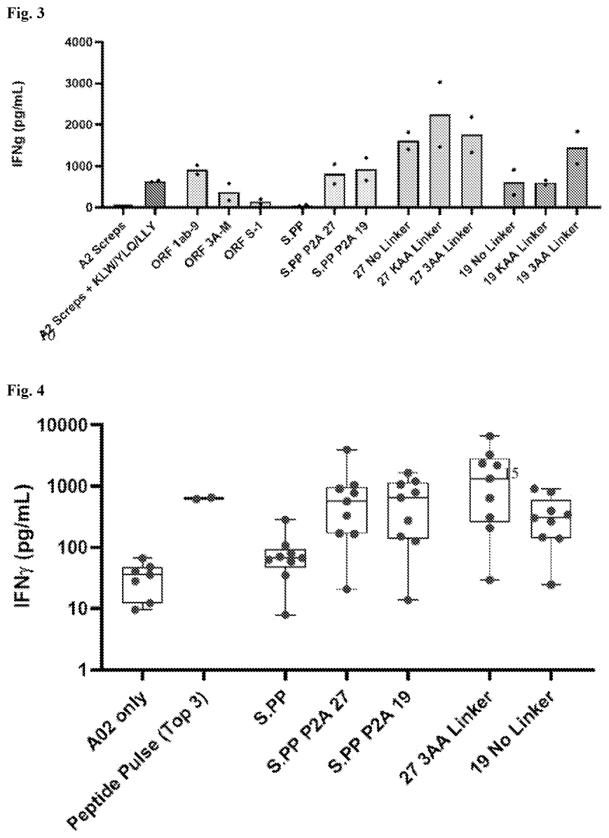

The results from the experiments are shown in FIG. 3 and FIG. 4 and several key results were determined.

First, it was observed that there were significant differences between the efficiency of epitope presentation depending on the context of each vaccine. In particular, the use of the three amino acids upstream and downstream of each epitope was identified as superior to the absence of linkers or the use of the KAA linker. Second, it was observed that the cytoplasmic expression of the constructs is critical. Indeed, very weak responses to the full-length S protein were observed in any of the patients, despite the fact that the S protein contained one of the immunodominant HLA-A*02:01 epitopes. In fact, the expression of a 500 aa fragment from the S protein that spanned this epitope (“ORF S-1”) resulted in a stronger response, thereby highlighting that the full-length S protein results in particularly poor presentation. Third, it was observed that the direct expression of the polyepitope vaccine results in stronger expression than the use of a P2A sequence. In general, the expression from 27 epitopes resulted in a stronger response than expression from 19 epitopes.

The most striking takeaway, however, was the efficiency of the vaccine constructs as a whole. All of the polyepitope constructs tested resulted in dramatically more robust T cell activity than the S protein alone, and several of the optimal constructs performed better than the positive control peptide pulsing. This result was very surprising, and emphasizes the potential of polyepitope vaccines when using the proper epitopes, linkers, and protein context.

These results lie in stark contrast to the state of the art, which has been skeptical of the utility of polyepitope constructs. For example, Korber et al. (2009) J. Virol. (2009) 83: 8300-8314 provides a broad overview of T-cell based vaccine approaches for HIV, and at its section on polyepitope vaccines highlights the lack of immunogenicity observed by this theoretical approach.

Based on the studies undertaken, the following two have been found to be important. First is the identity of the epitopes themselves. The included epitopes are those that have the highest level of functional validation—they are immunodominant in the context of actual SARS-CoV-2 infection. Other epitopes have been identified that are either predicted, detected on infected cells by mass spectrometry, or recognized by convalescent patients following sensitive antigen-specific expansion; this does not show that they are immunodominant (and therefore likely to be the most immunogenic and efficiently recognized by the immune system in the context of a vaccine). Second is the number of included epitopes. In some embodiments, epitopes are chosen to cover a broad diversity of HLA types (e.g., at least one immunodominant epitope per each of the 6 most common HLAs). It is noted that there are some epitopes that are presented by multiple HLA alleles, so it is possible to obtain a single immunodominant epitope on each of 6 alleles with only 4 or 5 epitopes. A minimum coverage of at least one immunodominant epitope per each of the 6 most common HLAs for example, could thus be achieved with 4, 6, or more epitopes, thereby providing a reasonable coverage of the HLA diversity in the population—vaccines with fewer than such HLA diversity coverage would have large blind spots and miss patients with suboptimal HLA alleles. More broadly, the value of the vaccine is believed to significantly increase by adding more epitopes. In some embodiments, the constructs have at least two immunodominant epitopes per each of the 6 most common HLAs. The constructs tested have a minimum of 18 epitopes (roughly 3 on each of 6 HLA alleles), and this is believed to increase the generation of a robust and broad response.

Having multiple epitopes per HLA allele is believed to be important because 1) it is not always known which epitopes (or proteins) are most protective, 2) it gives a higher likelihood that a robust T cell response would be raised against at least one of the included epitopes, and 3) a T cell response against multiple epitopes is important for efficacy and to prevent antigen escape variants.

The following factors were determined to have measureable, but relatively less, importance.

One factor is the identity of the linkers between the epitopes. While various linkers showed some efficacy, the ˜2-fold enhancement in epitope presentation seen with the 3 amino acid linker is meaningful, although longer linkers are suitable as well.

Another factor is the order of the epitopes. The order of the epitopes was optimized to limit junctional epitopes, but it is believed that constructs having various epitope orderings showed comparable results. There is not a hard cutoff for what kind of junctional epitope is believed to be problematic. In general, high-affinity junctional epitopes are believed to compete with desired epitopes and thereby reduce the magnitude of the observed immune response. For the vaccine constructs, all junctional epitopes with a predicted binding affinity <77 nM (such as ≤75 nM, ≤70 nM, ≤65 nM, ≤60 nM, ≤55 nM, ≤50 nM, ≤45 nM, ≤40 nM, ≤35 nM, ≤30 nM, ≤25 nM, ≤20 nM, ≤15 nM, ≤10 nM, ≤5 nM, or less, or any range in between, inclusive, such as 50-75 nM) were removed. In the case of the disclosed 29 epitope vaccine, this means that the 22 highest-affinity predicted binders across the entire construct are particularly desired epitopes. It is believed that a single epitope with predicted high-affinity binding would not have a dramatic effect, and generally the benefit of removing junctional epitopes is difficult to measure.

In addition, studies in relevant contexts suggests that the orientation has little effect on the efficiency of presentation. In theory, epitopes at the N-terminus a fragment could be presented more efficiently since the N terminus will always be synthesized, whereas some defective protein products may lack the C terminus. However, the experimental data suggest generally comparable presentation of the epitopes in a variety of orientations.

Due to the size constraints on an overall vaccine construct, there is a tradeoff between adding epitope repetitions versus including additional novel epitopes. Priority was given to additional novel epitopes to give broader HLA coverage and more epitopes (providing coverage of more viral proteins).

The 3aa linker appeared to be relatively the most effective from the preliminary analysis. Longer linkers are likely to be as effective as 3aa, but they require a larger overall vaccine construct and even the no-linker vaccines showed some efficacy. Thus, having a linker overall is helpful.

In some embodiments, a ribosomal stop/restart site is preferred since it is most robust and smallest. An IRES or a post-translational cleavage sequence could also be used in certain embodiments. In some embodiments, the S protein can be co-expressed to enable antibody responses alongside the polyepitope vaccine. It is believed that the two proteins could be expressed in either order. Typically, the first protein will have higher levels of expression, and having higher expression of the S protein is likely more important (if only to make for a more direct comparison to the S protein alone vaccines). The vaccine would be expected to work in either orientation.

An alternative to polycistronic expression in general is to deliver two vaccines simultaneously (especially easy to do with a delivery system like mRNA).

Various detection methods can be used in these studies. For example, tetramer staining can be used to quantify T cells against individual epitopes, T cell activation assays (e.g., CD137 staining, intracellular IFNg staining, etc.) to quantify reactive T cells, and TCR sequencing to demonstrate that the vaccine recapitulates the TCR repertoire raised in natural infection.

### Example 2: Additional SARS-CoV-2 Vaccine Constructs and Confirmatory Results

In further confirmation of the results described above, additional SARS-CoV-2 vaccine constructs were designed, such as those described in FIGS. 9C-9E.

Additional analyses were also performed to further confirm the results described above. For example, a variety of in vitro analyses were conducted using vaccine constructs formulated as lipid nanoparticles (LNPs). FIG. 10 shows that memory T cells isolated from SARS-CoV-2 patients react to cells treated with representative LNP formulations of the vaccine construct described in FIG. 9C, thereby demonstrating that cells can effectively process and present epitopes so that they can be recognized in a similar manner to cells infected by SARS-CoV-2. Briefly, memory T cells were isolated from recently convalesced SARS-CoV-2 patients (Tmem pools) using a CD8+ Memory T Cell Isolation Kit (Miltenyi, cat. #130-094-412) following the manufacturer's instructions. Tmem pools were co-cultured with HLA class I-null HEK293T cells expressing either HLA-A*02:01 or HLA-B*07:02 that were treated with 1 ug/mL mRNA-LNP complexes of the construct in FIG. 9C. After 24 hours, interferon gamma was measured using Human IFN-γ 3rd Generation Simple Plex Ella Assay (Protein Simple, cat. #SPCKB-PS-002574) according to the manufacturer's instructions.

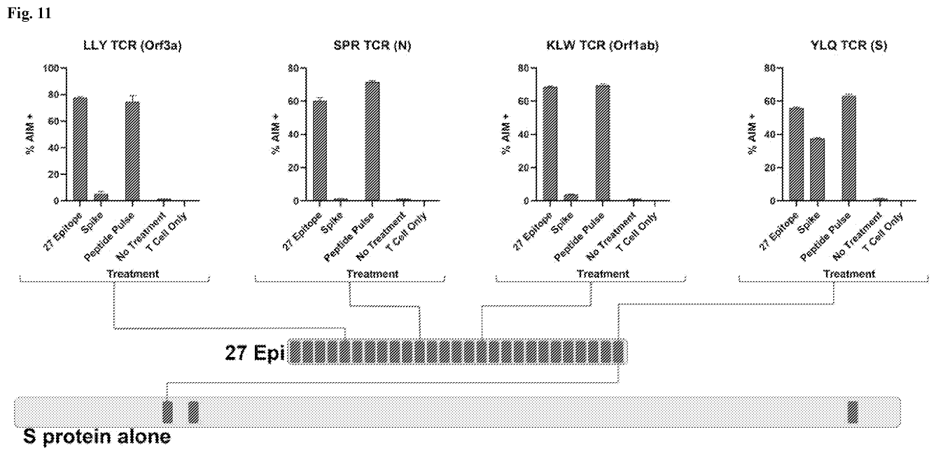

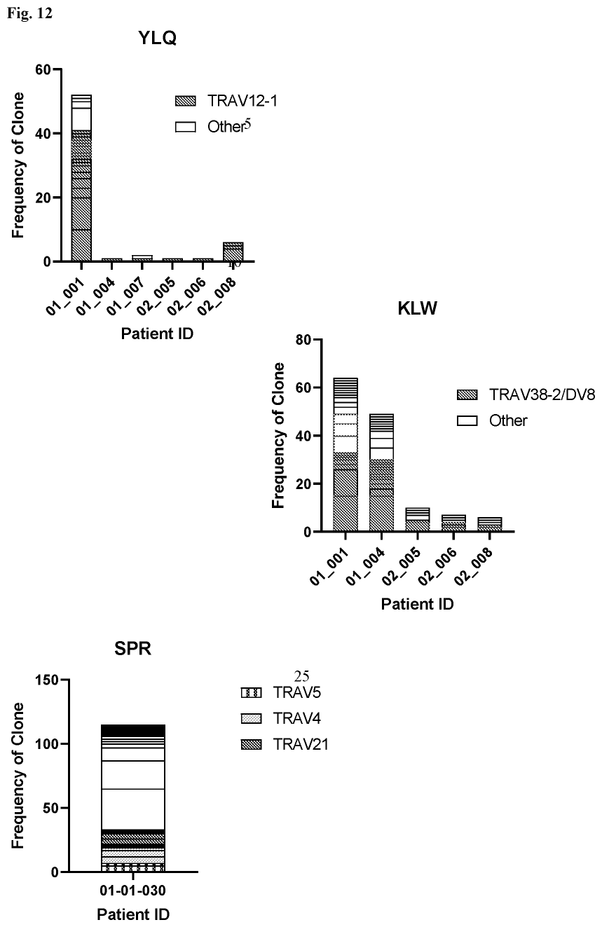

Moreover, individual TCR clones can be used as reagents to assess processing and presentation of specific epitopes (such as those representative clones and data shown in FIG. 11). For example, FIG. 11 shows that four individual epitopes contained in the 27 epitope construct described in FIG. 6A are processed and presented in a manner capable of being recognized by TCRs specific for epitopes of SARS-CoV-2. Briefly, monocytes were isolated using EasySep™ Human CD14 Positive Selection Kit II (StemCell Technologies, 17858) from HLA-A*02:01 and HLA-B*07:02 positive healthy donors and cultured in the presence of interleukin-4 and granulocyte-macrophage colony stimulating factor for 48 hours to differentiate to monocyte-derived dendritic cells (moDCs). MoDCs were treated with LNPs containing 1 ug/mL mRNA from the construct described in FIG. 6A for four hours, then treated with TNFalpha, IL-1beta, IL-6 and PGE2. After 48 hours, moDCs were co-cultured with T cells transduced with individual TCRs that each recognize either the LLY, SPR, KLW, or YLQ epitopes described in Table 1A and Table 1F. Activation of the TCR was measured by staining with AF647-conjugated anti-CD69 (Biolegend, cat. #310918) and PE-conjugated anti-CD137 (Biolegend, cat. #309804) antibodies detected by flow cytometry (Cytoflex S, Beckman Coulter).

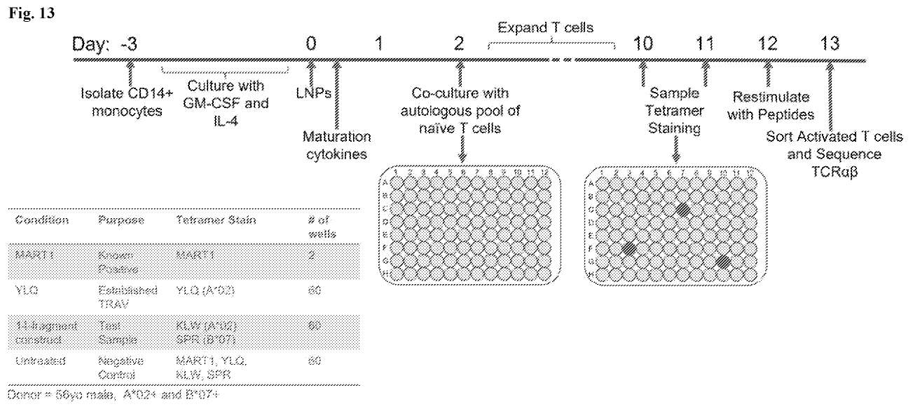

Interestingly, it was also determined that the LNP formulation of the vaccine construct described in FIG. 9C generates SARS-CoV-2 epitope-specific TCRs utilizing common TRAV genes (FIG. 13-16).

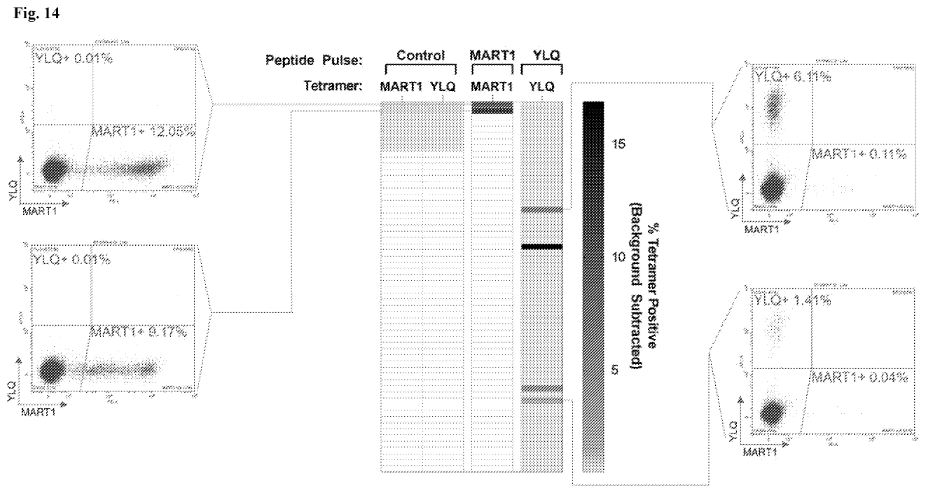

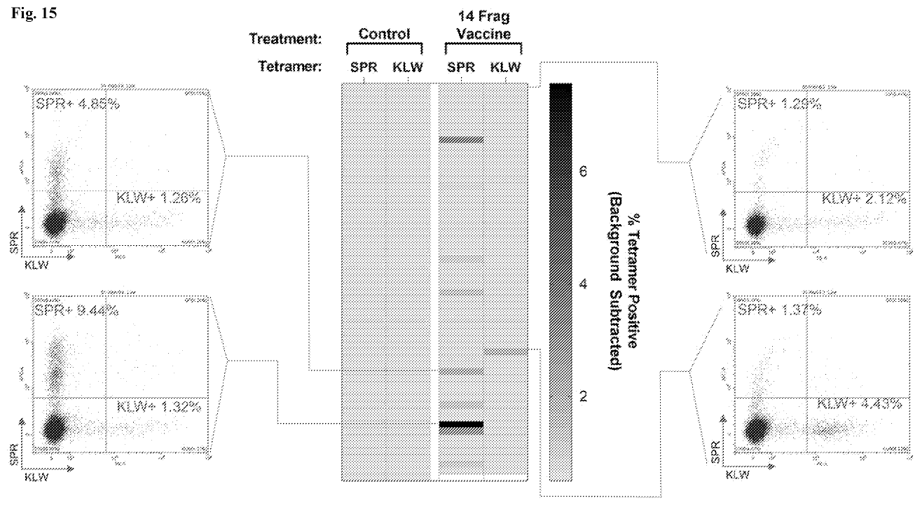

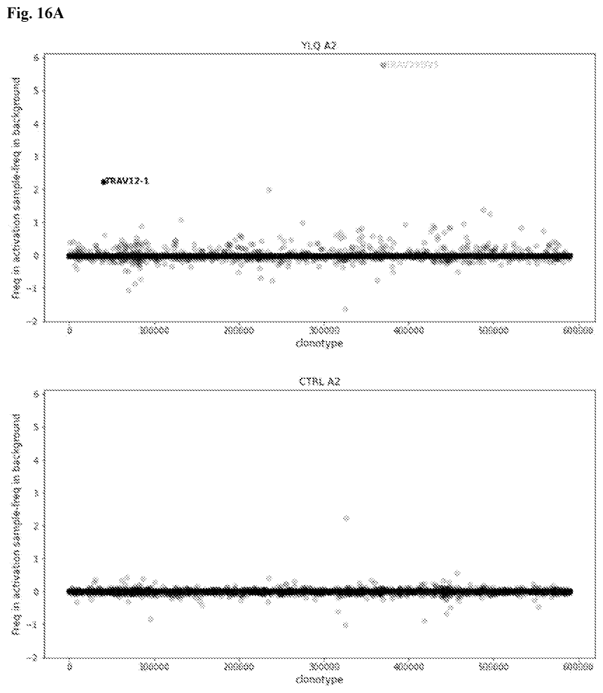

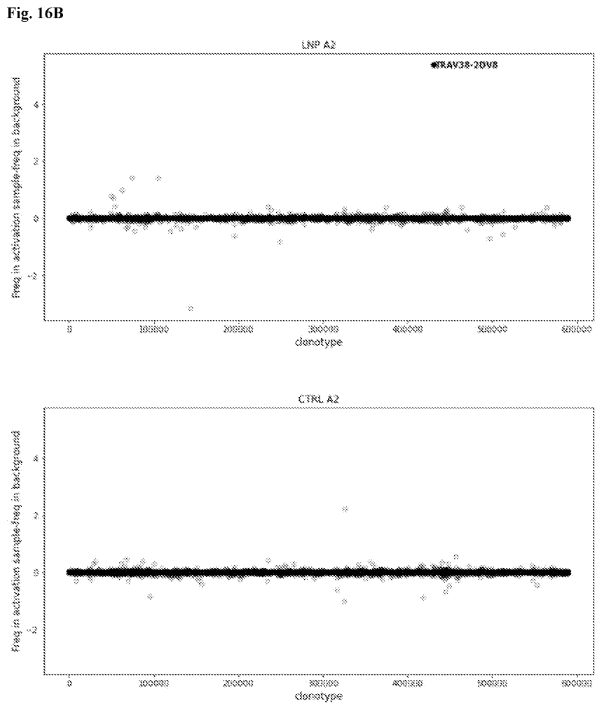

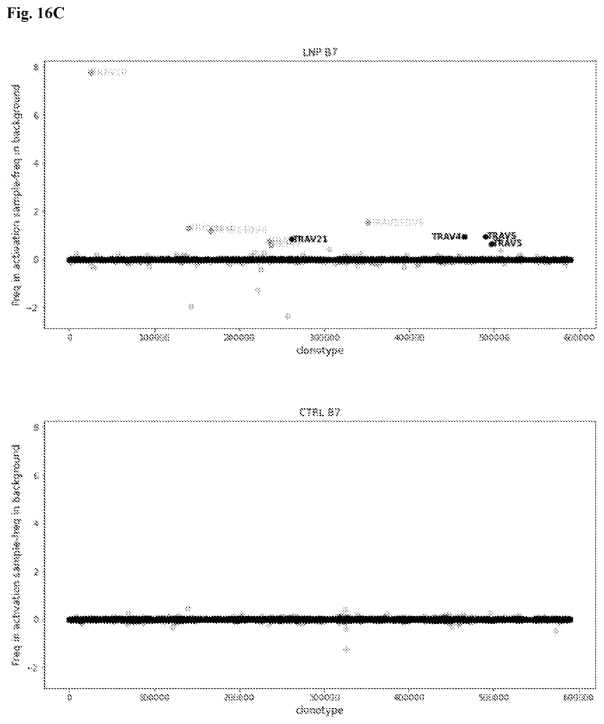

In response to natural infection with SARS-CoV-2, TCRs recognizing the same immunodominant epitope of SARS-CoV-2 share common TRAV genes and are the dominant TRAV genes utilized by memory T cells (Tmem) of SARS-CoV-2 patients shortly after recovering from infection (FIG. 12). In order to confirm that vaccine constructs expand T cells from a naïve repertoire that use the same TCR genes as Tmem cells from COVID patients, an in vitro vaccine model was performed (FIG. 13). Briefly, moDCs treated with LNPs containing 1 ug/mL mRNA from the construct described in FIG. 9C were co-cultured with naïve CD8 T cells isolated from healthy donors blood collected in 2019 before widespread infection of SARS-CoV-2 using EasySep™ Human Naïve CD8+ T Cell Isolation Kit II (StemCell, 17968). The co-cultures were split across multiple wells of a 96 well plate to detect the expansion of specific clones from the naïve repertoire. After 10 days of expansion, each well was split into 4 copies and each well of one copy was stained with fluorescently labeled tetramers for the indicated peptides (Tetramer Shop, cat. #HA02-070 and HB07-017) and measured by flow cytometry (Cytoflex S, Beckman Coulter). The remaining copies were pooled and restimulated with either HLA-A*02:01 or HLA-B*07:02 mono-allelic HEK293T cells pulsed with 1 ug/mL of each peptide from Table 1A and Table 1F respectively (FIG. 13). Re-activated T cells identified by CD69 and CD137 staining were sorted by flow cytometry (MoFlo Astrios EQ) and sequenced to determine the TRAV gene utilized by the expanded T cells. The results demonstrate that a pulsed SARS-CoV-2 immunodominant peptide, such as the YLQ peptide, induces an expansion of peptide-specific T cells in the assay (FIG. 14) and that the vaccine construct described in FIG. 9C induces expansion of SARS-CoV-specific T cells in the assay (FIG. 15). Interestingly, it was also determined that the LNP formulation of the vaccine construct described in FIG. 9C generates SARS-CoV-2 epitope specific TCRs utilizing common TRAV genes (FIG. 16).

Additional confirmatory experiments may be performed.

In one representative embodiment, a processing and presentation assay is performed. For example, a construct (e.g., formulated as a lipid nanoparticle encapsulating mRNA (mRNA-LNPs)) is introduced to dendritic cells (DCs), which are target cells for the construct. Treated DCs are co-cultured with epitope-specific T cells and reactivity of the T cell is measured, such as by analysis of surface activation markers and IFNg. The Eeitopes are likely being processed and presented appropriately when T cells responds. To compare the epitope processing and presentation of constructs, monocytes are isolated from healthy donors with the HLA type matching the epitopes of interest using a EasySep™ Human CD14 Positive Selection Kit II (StemCell Technologies, 17858). Monocytes are cultured for 72 hrs in the presence of granulocyte colony stimulating factor (GM-CSF) and interleukin 4 (IL-4) to differentiate to moDCs. After 72 hours moDCs are treated with LNPs containing 1 ug/mL mRNA of each construct for four hours and then treated with TNFalpha, IL-1beta, IL-6 and PGE2. After 48 hours, T cells transduce with epitope specific TCRs are added to the culture at a ratio of 1:1 moDCs to T cells and co-cultured for 24 hours. T cell activation is measured by flow cytometric staining of the AIMs CD69 (Biolegend, cat. #309804) and CD137 (Biolegend, cat. #309804) and measured by flow cytometry (Cytoflex S, Beckman Coulter). Constructs able to elicit activation of epitope specific T cells show that the epitope recognized by the TCR is processed and presented sufficiently to induce T cell activation. Further, when comparing the processing and presentation of a single epitope between constructs, a higher level of T cell activation elicited by a particular construct is interpreted as a higher level of processing and presentation for the epitope measured.

In another representative embodiment, an in vitro vaccine assay is performed. For example, a construct (e.g., mRNA-LNPs formulation) is again introduced to DCs mRNA-LNPs and the DCs are co-cultured with naive T cells from donor blood collected in 2019 or earlier (i.e., non-COVID exposed). The co-cultured cells are split among hundreds of wells of a 96-well plate and antigen-specific expansion is measured by peptide-conjugated MHC tetramer staining. The magnitude of the response is measured by the number of wells that are identified with an antigen-specific clone (FIG. 13). Following an expansion of 10 days, the T cells are pooled and re-activated with HEK cells expressing a single HLA of interest treated again with LNPs with 1 ug/mL mRNA of the construct used to elicit the initial response. The cells are stained with the AIMs CD69 (Biolegend, cat. #309804) and CD137 (Biolegend, cat. #309804) and AIM double-positive cells are sorted and the rearranged TCR genes are sequenced. The assay may compare the T cells that respond to the co-culture with the memory T cells from COVID patients and the repertoire of expanded T cells are analyzed.

In still another representative embodiment, an MHC expansion assay is performed. For example, a construct (e.g., mRNA-LNPs formulation) is introduced to cells, such as human embryonic kidney (HEK) cells, expressing a single HLA matching an HLA of COVID patient memory T cells in order to determine whether the immunogenic regions in SARS-CoV-2 proteins, such as N, Orf3a, and M, contain epitopes that are presented on additional HLAs so that the vaccination will be effective in individuals having HLAs other than those described above. To assess the theoretical percentage of people who will respond to the constructs, CD8 Tmem cells are isolated and banked from patients recently recovered from SARS-CoV-2 infection using a CD8+ Memory T Cell Isolation Kit (Miltenyi, cat. #130-094-412) and who express HLAs other than the ones of know epitopes described in Table 1. The Tmem cells are co-cultured with mono-allelic HEK cells expressing matched HLAs and T cell activation is determined by measuring IFNgamma release using Human IFN-γ 3rd Generation Simple Plex Ella Assay (Protein Simple, cat. #SPCKB-PS-002574). Because the HEKs treated with the constructs contain a single HLA, activation of the Tmem cells shows that epitopes on the construct are processed and presented on the tested HLA. Further, recognition of COVID patient Tmem cells illustrates that the undefined epitope was sufficient to generate specific T cells in the memory repertoire of patients exposed to SARS-CoV-2 and patients harboring the tested HLA will likely be capable of generating a T cells response to the epitope delivered by the construct.

In yet another representative embodiment, an in vivo vaccine assay is performed. For example, animal models (e.g., humanized mouse models engineered to express a human TCR repertoire and human MHCs such as, for example, a VELOCI-T® mouse model; Regeneron, Inc.) and human subjects may be immunized with constructs to determine anti-SARS-CoV-2 immunity and epitope specific T cell responses may be measured using peptide-conjugated MHC tetramer staining, ELISPOT assays, or co-culture assays where Tmem cells from vaccinated mice or patients are co-cultured with mono-allelic HEKs treated with LNPs carrying mRNA of the corresponding vaccine construct.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments encompassed by the present invention described herein. Such equivalents are intended to be encompassed by the following claims.

What is claimed is:

1. An immunogenic polypeptide comprising at least two peptide epitopes selected from Table 1A, 1B, 1C, 1D, 1E, and/or 1F.

2. The immunogenic polypeptide of claim 1, comprising said at least two peptide epitopes in a concatenated order, optionally wherein at least one or more immunodominant epitopes are present in more than one copy.

3. The immunogenic polypeptide of claim 2, comprising at least 3, 4, 5, 6, 7, 8, 9, 10, 11, 12, 13, 14, 15, 16, 17, 18, 19, 20, 21, 22, 23, 24, 25, 26, 27, 28, 29, or more of said peptide epitopes, optionally wherein the immunogenic polypeptide comprises at least one, two, and/or three immunodominant epitopes per each of HLA-A*02, HLA-A*03, HLA-A*01, HLA-A*11, HLA-A*24, and HLA-B*07.

4. The immunogenic polypeptide of claim 2 or 3, comprising 3 peptide epitopes from each of Table 1A, 1B, 1C, 1D, 1E, and 1F.

5. The immunogenic polypeptide of any one of claims 2 to 4, further comprising a linker between the peptide epitopes.

6. The immunogenic polypeptide of claim 5, wherein the linker comprises at least three amino acids for each of the peptide epitopes, wherein said at least three amino acids are those that are contiguous with their respective peptide epitopes.

7. The immunogenic polypeptide of claim 5, wherein the linker is a proteasomal cleavage motif.

8. The immunogenic polypeptide of any one of claims 2 to 7, further comprising one or more full-length SARS-CoV-2 proteins selected from the group consisting of Orf1ab, M, N, Orf3a, and S, or one or more protein fragments thereof, optionally wherein the fragments of the one or more full-length SARS-CoV-2 protein(s) encompass the SARS-CoV-2 protein(s) without encoding the functional SARS-CoV-2 protein(s).

9. The immunogenic polypeptide of any one of claims 2 to 8, further comprising a ribosomal stop/restart segment, IRES segment, and/or a post-translational cleavage segment, optionally wherein the post-translational cleavage segment is a P2A segment.

10. The immunogenic polypeptide of any one of claims 2 to 9, comprising any one of the amino acid sequences provided in Table 1G or Table 11.

11. The immunogenic polypeptide of claim 1, comprising at least two peptide fragments each of which comprises at least two of said peptide epitopes, wherein said at least two of said peptide epitopes in each peptide fragment are derived from the same protein of SARS-CoV-2.

12. The immunogenic polypeptide of claim 11, wherein said at least two peptide fragments are derived from the N protein, the M protein, the ORF1a/b protein, or the ORF3a protein of SARS-CoV-2.

13. The immunogenic polypeptide of claim 11 or 12, comprising at most 6 of said peptide fragments.

14. The immunogenic polypeptide of any one of claims 11 to 13, further comprising one or more full-length SARS-CoV-2 proteins selected from the group consisting of Orf1ab, M, N, Orf3a, and S, or one or more protein fragments thereof, optionally wherein the fragments of the one or more full-length SARS-CoV-2 protein(s) encompass the SARS-CoV-2 protein(s) without encoding the functional SARS-CoV-2 protein(s).

15. The immunogenic polypeptide of any one of claims 11 to 14, further comprising a ribosomal stop/restart segment, IRES segment, and/or a post-translational cleavage segment, optionally wherein the post-translational cleavage segment is a P2A segment.

16. The immunogenic polypeptide of any one of claims 11, 12, 14, and 15, comprising any one of the amino acid sequences provided in Table 1H or Table 1J.

17. The immunogenic polypeptide of any one of claims 1 to 16, wherein the immunogenic polypeptide is capable of eliciting a T cell response in vitro and/or in vivo, optionally wherein the T cell response is determined by a tetramer staining assay, T cell activation assay, CD137 staining assay, intracellular IFNg staining assay, cytokine release assay, and/or T cell proliferation assay.

18. An immunogenic composition comprising at least one immunogenic polypeptide of any one of claims 1-17, optionally wherein the immunogenic composition further comprises 1) one or more full-length SARS-CoV-2 proteins selected from the group consisting of Orf1ab, M, N, Orf3a, and S, or one or more protein fragments thereof, optionally wherein the fragments of the one or more full-length SARS-CoV-2 protein(s) encompass the SARS-CoV-2 protein(s) without encoding the functional SARS-CoV-2 protein(s) and/or 2) an adjuvant.

19. The immunogenic composition of claim 18, wherein the immunogenic composition is capable of eliciting a T cell response in vitro and/or in vivo, optionally wherein the T cell response is determined by a tetramer staining assay, T cell activation assay, CD137 staining assay, intracellular IFNg staining assay, cytokine release assay, and/or T cell proliferation assay.

20. The immunogenic composition of claim 18 or 19, wherein the immunogenic composition is capable of eliciting a T cell response in a subject.

21. A composition comprising at least one immunogenic polypeptide of any one of claims 1-17 and an MHC molecule.

22. The composition of claim 21, wherein the MHC molecule is an MHC multimer, optionally wherein the MHC multimer is a tetramer.

23. The composition of claim 21 or 22, wherein the MHC molecule is an MHC class I molecule.

24. The composition of any one of claims 21-23, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele.

25. A stable MHC-peptide complex, comprising a peptide epitope of said at least one immunogenic polypeptide of any one of claims 1-17 in the context of an MHC molecule.

26. The stable MHC-peptide complex of claim 25, wherein the MHC molecule is an MHC multimer, optionally wherein the MHC multimer is a tetramer.

27. The stable MHC-peptide complex of claim 25 or 26, wherein the MHC molecule is an MHC class I molecule.

28. The stable MHC-peptide complex of any one of claims 25-27, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele.

29. The stable MHC-peptide complex of any one of claims 25-28, wherein the peptide epitope and the MHC molecule are covalently linked and/or wherein the alpha and beta chains of the MHC molecule are covalently linked.

30. The stable MHC-peptide complex of any one of claims 25-29, wherein the stable MHC-peptide complex comprises a detectable label, optionally wherein the detectable label is a fluorophore.

31. An immunogenic composition comprising the stable MHC-peptide complex of any one of claims 25-30, and an adjuvant.

32. An isolated nucleic acid that encodes the immunogenic polypeptide of any one of claims 1-17, or a complement thereof, optionally wherein the isolated nucleic acid is DNA, RNA, chemically modified RNA, mRNA, cDNA, self-replicating, cyclized, concatamerized, comprises a 5′ untranslated region (5′UTR) and/or 3′UTR, comprises an expression promoter, comprises an internal ribosome entry site (IRES), and/or comprises a self-cleaving 2A peptide, such as P2A or T2A.

33. A vector comprising the isolated nucleic acid of claim 32, optionally wherein the vector is an expression vector.

34. A cell that a) comprises the isolated nucleic acid of claim 32, b) comprises the vector of claim 33, and/or c) produces one or more immunogenic polypeptides of any one of claims 1-17 and/or presents at the cell surface one or more stable MHC-peptide complexes of any one of claims 25-30, optionally wherein the cell is genetically engineered.

35. A binding moiety that specifically binds an immunogenic polypeptide of any one of claims 1-17 and/or the stable MHC-peptide complex of any one of claims 25-30, optionally wherein the binding moiety is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

36. A device or kit comprising a) one or more immunogenic polypeptides of any one of claims 1-17 and/or b) one or more stable MHC-peptide complexes of any one of claims 25-30, said device or kit optionally comprising a reagent to detect binding of a) and/or b) to a T cell receptor.

37. A method of detecting T cells that bind a stable MHC-peptide complex comprising:
a) contacting a sample comprising T cells with a stable MHC-peptide complex of any one of claims 25-30; and
b) detecting binding of T cells to the stable MHC-peptide complex, optionally further determining the percentage of stable MHC-peptide-specific T cells that bind to the stable MHC-peptide complex, optionally wherein the sample comprises peripheral blood mononuclear cells (PBMCs).

38. The method of claim 37, wherein the T cells are CD8+ T cells.

39. The method of claim 37 or 38, wherein the detecting and/or determining is performed using fluorescence activated cell sorting (FACS), enzyme linked immunosorbent assay (ELISA), radioimmune assay (RIA), immunochemically, Western blot, or intracellular flow assay.

40. The method of any one of claims 37-39, wherein the sample comprises T cells contacted with, or suspected of having been contacted with, one or more SARS-CoV-2 proteins or fragments thereof.

41. A method of determining whether a subject has exposure to and/or protection from SARS-CoV-2 comprising:
a) incubating a cell population comprising T cells obtained from the subject with an immunogenic polypeptide of any one of claims 1-17 or a stable MHC-peptide complex of any one of claims 25-30; and
b) detecting the presence or level of reactivity,
wherein the presence of or a higher level of reactivity compared to a control level indicates that the subject has exposure to and/or protection from SARS-CoV-2.

42. A method for predicting the clinical outcome of a subject afflicted with SARS-CoV-2 infection comprising:
a) determining the presence or level of reactivity between T cells obtained from the subject and one more immunogenic polypeptides of any one of claims 1-17 or one or more stable MHC-peptide complexes of any one of claims 25-30; and
b) comparing the presence or level of reactivity to that from a control, wherein the control is obtained from a subject having a good clinical outcome;
wherein the presence or a higher level of reactivity in the subject sample as compared to the control indicates that the subject has a good clinical outcome.

43. A method of assessing the efficacy of a SARS-CoV-2 therapy comprising:
a) determining the presence or level of reactivity between T cells obtained from the subject and one more immunogenic polypeptides of any one of claims 1-17 or one or more stable MHC-peptide complexes of any one of claims 25-30, in a first sample obtained from the subject prior to providing at least a portion of the SARS-CoV-2 therapy to the subject, and
b) determining the presence or level of reactivity between the one more immunogenic polypeptides of any one of claims 1-17, or the one or more stable MHC-peptide complexes of any one of claims 25-30, and T cells obtained from the subject present in a second sample obtained from the subject following provision of the portion of the SARS-CoV-2 therapy,
wherein the presence or a higher level of reactivity in the second sample, relative to the first sample, is an indication that the therapy is efficacious for treating SARS-CoV-2 in the subject.

44. The method of any one of claims 41-43, wherein the level of reactivity is indicated by a) the presence of binding and/or b) T cell activation and/or effector function, optionally wherein the T cell activation or effector function is T cell proliferation, killing, or cytokine release.

45. The method of any one of claims 41-44, further comprising repeating steps a) and b) at a subsequent point in time, optionally wherein the subject has undergone treatment to ameliorate SARS-CoV-2 infection between the first point in time and the subsequent point in time.

46. The method of any one of claims 41-45, wherein the T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

47. The method of any one of claims 41-46, wherein the control level is a reference number.

48. The method of any one of claims 41-47, wherein the control level is a level of a subject without exposure to SARS-CoV-2.

49. A method of preventing and/or treating SARS-CoV-2 infection in a subject comprising administering to the subject a therapeutically effective amount of an immunogenic composition comprising and/or encoding at least one immunogenic polypeptide of any one of claims 1-17, or a cell of claim 34.

50. The method of claim 49, wherein the immunogenic composition comprises a nucleic acid that encodes an immunogenic polypeptide of any one of claims 1-17.

51. The method of claim 49 or 50, wherein the nucleic acid is DNA, RNA, mRNA, cDNA, self-replicating, cyclized, and/or concatamerized.

52. The method of any one of claims 49-51, wherein the SARS-CoV-2 protein is selected from the group consisting of orf1a/b, S protein, N protein, M protein, orf3a, and orf7a.

53. The method of any one of claims 49-51, wherein the immunogenic polypeptide is capable of eliciting a T cell response in a subject.

54. The method of any one of claims 49-52, wherein the immunogenic composition comprises more than one immunogenic polypeptide.

55. The method of any one of claims 49-54, wherein the immunogenic composition further comprises an adjuvant.

56. The method of any one of claims 49-55, wherein the immunogenic composition is capable of eliciting a T cell response in a subject.

57. The method of any one of claims 49-56, wherein the administered immunogenic composition induces an immune response against the SARS-CoV-2 in the subject.

58. The method of any one of claims 49-57, wherein the administered immunogenic composition induces a T cell immune response against the SARS-CoV-2 in the subject.

59. The method of any one of claims 49-58, wherein the T cell immune response is a CD8+ T cell immune response.

60. A method of identifying a peptide-binding molecule, or antigen-binding fragment thereof, that binds to a peptide epitope of said at least one immunogenic polypeptide of any one of claims 1-17, comprising:
a) providing a cell presenting a peptide epitope of said at least one immunogenic polypeptide of any one of claims 1-17 in the context of an MHC molecule on the surface of the cell, optionally, wherein the cell comprises a nucleic acid encoding and expressing the at least one immunogenic polypeptide;
b) determining binding of a plurality of candidate peptide-binding molecules or antigen-binding fragments thereof to the peptide epitope in the context of the MHC molecule on the cell; and
c) identifying one or more peptide-binding molecules or antigen-binding fragments thereof that bind to the peptide epitope in the context of the MHC molecule.

61. The method of claim 60, wherein the step a) comprises contacting the MHC molecule on the surface of the cell with a peptide epitope selected from Table 1A, 1B, 1C, 1D, 1E, and/or 1F.

62. The method of claim 60, wherein the step a) comprises transfecting the cell with a basic nucleic acid and/or a vector comprising the basic nucleic acid, wherein

63. A method of identifying a peptide-binding molecule or antigen-binding fragment thereof that binds to a peptide epitope of said at least one immunogenic polypeptide of any one of claims 1-17, comprising:
a) providing a stable MHC-peptide complex comprising a peptide epitope of said at least one immunogenic polypeptide of any one of claims 1-17 in the context of an MHC molecule;
b) determining binding of a plurality of candidate peptide-binding molecules or antigen-binding fragments thereof to the stable MHC-peptide complex; and
c) identifying one or more peptide-binding molecules or antigen-binding fragments thereof that bind to the stable MHC-peptide complex.

64. The method of claim 63, wherein the MHC molecule is a MHC multimer, optionally wherein the MHC multimer is a tetramer.

65. The method of claim 63 or 64, wherein the MHC molecule is a MHC class I molecule.

66. The method of any one of claims 63-65, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele.

67. The method of any one of claims 63-66, wherein the peptide epitope and the MHC molecule are covalently linked and/or wherein the alpha and beta chains of the MHC molecule are covalently linked.

68. The method of any one of claims 63-67, wherein the stable MHC-peptide complex comprises a detectable label, optionally wherein the detectable label is a fluorophore.

69. The method of any of claims 60-68, wherein the plurality of candidate peptide binding molecules comprises one or more T cell receptors (TCRs), or one or more antigen-binding fragments of a TCR.

70. The method of any of claims 60-69, wherein the plurality of candidate peptide binding molecules comprises at least 2, 5, 10, 100, 10³, 10⁴, 10⁵, 10⁶, 10⁷, 10⁸, 10⁹, or more, different candidate peptide binding molecules.

71. The method of any of claims 60-70, wherein the plurality of candidate peptide binding molecules comprises one or more candidate peptide binding molecules that are obtained from a sample from a subject or a population of subjects; or the plurality of candidate peptide binding molecules comprises one or more candidate peptide binding molecules that comprise mutations in a parent scaffold peptide binding molecule obtained from a sample from a subject.

72. The method of claim 71, wherein the subject or population of subjects are a) not infected with SARS-CoV-2 and/or have recovered from COVID-19 orb) infected with SARS-CoV-2 and/or have COVID-19.

73. The method of any of claim 71 or 72, wherein the subject or population of subjects has been vaccinated with one or more immunogenic polypeptides, wherein the immunogenic polypeptides comprise a peptide epitope selected from Table 1A, 1B, 1C, 1D, 1E, and/or 1F.

74. The method of any of claims 68-73, wherein the subject is a mammal, optionally wherein the mammal is a human, a primate, or a rodent.

75. The method of any one of claims 71-74, wherein the subject is an HLA-transgenic mouse and/or is a human TCR transgenic mouse.

76. The method of any of claims 71-75, wherein the sample comprises T cells.

77. The method of claim 76, wherein the sample comprises peripheral blood mononuclear cells (PBMCs) or CD8+ memory T cells.

78. The peptide-binding molecule or antigen-binding fragment thereof identified according to any one of claims 60-77, optionally wherein the binding moiety is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

79. A method of treating SARS-CoV-2 infection in a subject comprising administering to the subject a therapeutically effective amount of genetically engineered T cells that express a TCR identified by the method of any one of claims 63-78.

80. A method of treating SARS-CoV-2 infection in a subject comprising administering to the subject a therapeutically effective amount of genetically engineered T cells that express a TCR that binds to a peptide epitope of said at least one immunogenic polypeptide of any one of claims 1-17.

81. A method of treating SARS-CoV-2 infection in a subject comprising administering to the subject a therapeutically effective amount of genetically engineered T cells that express a TCR that binds to a stable MHC-peptide complex comprising a peptide epitope of said at least one immunogenic polypeptide of any one of claims 1-17 in the context of an MHC molecule.

82. The method of claim 81, wherein the MHC molecule is a MHC multimer, optionally wherein the MHC multimer is a tetramer.

83. The method of any one of claims 79-82, wherein the MHC molecule is a MHC class I molecule.

84. The method of any one of claims 79-83, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele.

85. The method of any one of claims 79-84, wherein the peptide epitope and the MHC molecule are covalently linked and/or wherein the alpha and beta chains of the MHC molecule are covalently linked.

86. The method of any one of claims 79-85, wherein the stable MHC-peptide complex comprises a detectable label, optionally wherein the detectable label is a fluorophore.

87. The method of any one of claims 79-86, wherein the T cells are isolated from a) the subject, b) a donor not infected with SARS-CoV-2, or c) a donor recovered from COVID-19.

88. A method of preventing or treating SARS-CoV-2 infection in a subject comprising transfusing antigen-specific T cells to the subject, wherein the antigen-specific T cells are generated by:
a) stimulating PBMCs or T cells from a subject with an immunogenic polypeptide of any one of claims 1-17, a nucleic acid encoding an immunogenic polypeptide of any one of claims 1-17, a stable MHC-peptide complex comprising a peptide epitope of said at least one immunogenic polypeptide of any one of claims 1-17 in the context of an MHC molecule, or a cell that encodes and/or presents a peptide epitope of said at least one immunogenic polypeptide of any one of claims 1-17 in the context of a MHC molecule on its cell surface; and
b) expanding antigen-specific T cells in vitro, optionally isolating PBMCs or T cells from the subject before stimulating the PBMCs or T cells.

89. The method of claim 88, wherein the T cell is a naive T cell, a central memory T cell, or an effector memory T cell.

90. The method of claim 89, wherein the T cell is a CD8+ memory T cell.

91. The method of any one of claims 36-90, wherein the agents are placed in contact under conditions and for a time suitable for the formation of at least one immune complex between the peptide epitope, immunogenic polypeptide, stable MHC-peptide complex, T cell receptor, and/or T cell.

92. The method of any one of claims 36-91, wherein the peptide epitope, immunogenic polypeptide, stable MHC-peptide complex, and/or T cell receptor are expressed by cells and the cells are expanded and/or isolated during one or more steps.

93. The method of any one of claims 36-92, wherein the subject is a mammal, optionally wherein the mammal is a human, a primate, or a rodent.


SEQUENCE LISTING (65 sequences)

SEQ ID NO: 1 - protein, 9 aa, Severe acute respiratory syndrome coronavirus 2
KLWAQCVQL

SEQ ID NO: 2 - protein, 9 aa, Severe acute respiratory syndrome coronavirus 2
YLQPRTFLL

SEQ ID NO: 3 - protein, 9 aa, Severe acute respiratory syndrome coronavirus 2
LLYDANYFL

SEQ ID NO: 4 - protein, 9 aa, Severe acute respiratory syndrome coronavirus 2
ALWEIQQVV

SEQ ID NO: 5 - protein, 9 aa, Severe acute respiratory syndrome coronavirus 2
LLLDRLNQL

SEQ ID NO: 6 - protein, 11 aa, Severe acute respiratory syndrome coronavirus 2
YLFDESGEFKL

SEQ ID NO: 7 - protein, 9 aa, Severe acute respiratory syndrome coronavirus 2
FTSDYYQLY

SEQ ID NO: 8 - protein, 10 aa, Severe acute respiratory syndrome coronavirus 2
TTDPSFLGRY

SEQ ID NO: 9 - protein, 9 aa, Severe acute respiratory syndrome coronavirus 2
PTDNYITTY

SEQ ID NO: 10 - protein, 9 aa, Severe acute respiratory syndrome coronavirus 2
ATSRTLSYY

SEQ ID NO: 11 - protein, 10 aa, Severe acute respiratory syndrome coronavirus 2
CTDDNALAYY

SEQ ID NO: 12 - protein, 10 aa, Severe acute respiratory syndrome coronavirus 2
NTCDGTTFTY

SEQ ID NO: 13 - protein, 9 aa, Severe acute respiratory syndrome coronavirus 2
DTDFVNEFY

SEQ ID NO: 14 - protein, 9 aa, Severe acute respiratory syndrome coronavirus 2
GTDLEGNFY

SEQ ID NO: 15 - protein, 9 aa, Severe acute respiratory syndrome coronavirus 2
KTFPPTEPK

SEQ ID NO: 16 - protein, 9 aa, Severe acute respiratory syndrome coronavirus 2
KCYGVSPTK

SEQ ID NO: 17 - protein, 10 aa, Severe acute respiratory syndrome coronavirus 2
MVTNNTFTLK

SEQ ID NO: 18 - protein, 9 aa, Severe acute respiratory syndrome coronavirus 2
KTIQPRVEK

SEQ ID NO: 19 - protein, 9 aa, Severe acute respiratory syndrome coronavirus 2
VTDTPKGPK

SEQ ID NO: 20 - protein, 10 aa, Severe acute respiratory syndrome coronavirus 2
ATEGALNTPK

SEQ ID NO: 21 - protein, 9 aa, Severe acute respiratory syndrome coronavirus 2
ASAFFGMSR

SEQ ID NO: 22 - protein, 10 aa, Severe acute respiratory syndrome coronavirus 2
ATSRTLSYYK

SEQ ID NO: 23 - protein, 9 aa, Severe acute respiratory syndrome coronavirus 2
QYIKWPWYI

SEQ ID NO: 24 - protein, 9 aa, Severe acute respiratory syndrome coronavirus 2
VYFLQSINF

SEQ ID NO: 25 - protein, 9 aa, Severe acute respiratory syndrome coronavirus 2
VYIGDPAQL

SEQ ID NO: 26 - protein, 9 aa, Severe acute respiratory syndrome coronavirus 2
SPRWYFYYL

SEQ ID NO: 27 - protein, 8 aa, Severe acute respiratory syndrome coronavirus 2
RPDTRYVL

SEQ ID NO: 28 - protein, 9 aa, Severe acute respiratory syndrome coronavirus 2
IPRRNVATL

SEQ ID NO: 29 - protein, 288 aa, Artificial Sequence
  Description of Artificial Sequence Synthetic polypeptide
YASALWEIQQVVDADHSYFTSDYYQLYSTQKHYVYIGDPAQLPAPDAYKTFPPTEPKKDKIWVATEGALNTPKDHIITVATSRTLSYYKLGAYALVYFLQSINFVRISLEIPRRNVATLQAEPNMMVTNNTFTLKGGAKNPLLYDANYFLCWHKDLSPRWYFYYLGTGYYHTTDPSFLGRYMSAESLRPDTRYVLMDGRKVPTDNYITTYPGQCRFVTDTPKGPKVKYSTFKCYGVSPTKLNDKYEQYIKWPWYIWLGSSSKLWAQCVQLHNDYVGYLQPRTFLLKYN

SEQ ID NO: 30 - protein, 228 aa, Artificial Sequence
  Description of Artificial Sequence Synthetic polypeptide
ALWEIQQVVKAAFTSDYYQLYKAAVYIGDPAQLKAAKTFPPTEPKKAAATEGALNTPKKAAATSRTLSYYKKAAVYFLQSINFKAAIPRRNVATLKAAMVTNNTFTLKKAALLYDANYFLKAASPRWYFYYLKAATTDPSFLGRYKAARPDTRYVLKAAPTDNYITTYKAAVTDTPKGPKKAAKCYGVSPTKKAAQYIKWPWYIKAAKLWAQCVQLKAAYLQPRTFLL

SEQ ID NO: 31 - protein, 174 aa, Artificial Sequence
  Description of Artificial Sequence Synthetic polypeptide
ALWEIQQVVFTSDYYQLYVYIGDPAQLKTFPPTEPKATEGALNTPKATSRTLSYYKVYFLQSINFIPRRNVATLMVTNNTFTLKLLYDANYFLSPRWYFYYLTTDPSFLGRYRPDTRYVLPTDNYITTYVTDTPKGPKKCYGVSPTKQYIKWPWYIKLWAQCVQLYLQPRTFLL

SEQ ID NO: 32 - protein, 412 aa, Artificial Sequence
  Description of Artificial Sequence Synthetic polypeptide
HSYFTSDYYQLYSTQKHYVYIGDPAQLPAPYALVYFLQSINFVRIATYYLFDESGEFKLASHKNPLLYDANYFLCWHRKVPTDNYITTYPGQITVATSRTLSYYKLGAPNMMVTNNTFTLKGGASIIKTIQPRVEKKKLKYEQYIKWPWYIWLGKDLSPRWYFYYLGTGTYKNTCDGTTFTYASAVHAGTDLEGNFYGPFESLRPDTRYVLMDGQTACTDDNALAYYNTTSSSKLWAQCVQLHNDYASALWEIQQVVDADDAYKTFPPTEPKKDKSTFKCYGVSPTKLNDAPSASAFFGMSRIGMLALLLLDRLNQLESKSLEIPRRNVATLQAEYYHTTDPSFLGRYMSARDVDTDFVNEFYAYLCRFVTDTPKGPKVKYIWVATEGALNTPKDHIYVGYLQPRTFLLKYN

SEQ ID NO: 33 - protein, 328 aa, Artificial Sequence
  Description of Artificial Sequence Synthetic polypeptide
FTSDYYQLYKAAVYIGDPAQLKAAVYFLQSINFKAAYLFDESGEFKLKAALLYDANYFLKAAPTDNYITTYKAAATSRTLSYYKKAAMVTNNTFTLKKAAKTIQPRVEKKAAQYIKWPWYIKAASPRWYFYYLKAANTCDGTTFTYKAAGTDLEGNFYKAARPDTRYVLKAACTDDNALAYYKAAKLWAQCVQLKAAALWEIQQVVKAAKTFPPTEPKKAAKCYGVSPTKKAAASAFFGMSRKAALLLDRLNQLKAAIPRRNVATLKAATTDPSFLGRYKAADTDFVNEFYKAAVTDTPKGPKKAAATEGALNTPKKAAYLQPRTFLL

SEQ ID NO: 34 - protein, 250 aa, Artificial Sequence
  Description of Artificial Sequence Synthetic polypeptide
FTSDYYQLYVYIGDPAQLVYFLQSINFYLFDESGEFKLLLYDANYFLPTDNYITTYATSRTLSYYKMVTNNTFTLKKTIQPRVEKQYIKWPWYISPRWYFYYLNTCDGTTFTYGTDLEGNFYRPDTRYVLCTDDNALAYYKLWAQCVQLALWEIQQVVKTFPPTEPKKCYGVSPTKASAFFGMSRLLLDRLNQLIPRRNVATLTTDPSFLGRYDTDFVNEFYVTDTPKGPKATEGALNTPKYLQPRTFLL

SEQ ID NO: 35 - protein, 500 aa, Artificial Sequence
  Description of Artificial Sequence Synthetic polypeptide
YALVYFLQSINFVRIIMRLWLCWKCRSKNPLLYDANYFLCWHGDGKMKDLSPRWYFYYLGTGPEAGLPYGANKDGIIWVATEGALNTPKDHIGTRNPEKYCALAPNMMVTNNTFTLKGGAPTKVTFGTYKNTCDGTTFTYASALWEIQQVVDADLNESLIDLQELGKYEQYIKWPWYIWLGFIAGLIAIVMVQTACTDDNALAYYNTTKGGRFVLALLSDLQDLKWARFPKSDGTGTIYTELEPPCRFVTDTPKGPKVKYLRVEAFEYYHTTDPSFLGRYMSALNHTKKWITVATSRTLSYYKLGASQRVAGDSGFAAYSRYRIGNYKLNTDHSSSSDNIALLVQVLQQLRVESSSKLWAQCVQLHNDILLAKDTAPSASAFFGMSRIGMEVTPSGTWLTYTGAIKLDDKDPNFKDQVILLNKHIDAYKTFPPTEPKKDKSKLIEYTDFATSACVLAAECTIFKDASGKPVPYCYDTNVLEGSVAYESLRPDTRYVLMDG

SEQ ID NO: 36 - protein, 1588 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTRAKRSGSGATNFSLLKQAGDVEENPGPYASALWEIQQVVDADHSYFTSDYYQLYSTQKHYVYIGDPAQLPAPDAYKTFPPTEPKKDKIWVATEGALNTPKDHIITVATSRTLSYYKLGAYALVYFLQSINFVRISLEIPRRNVATLQAEPNMMVTNNTFTLKGGAKNPLLYDANYFLCWHKDLSPRWYFYYLGTGYYHTTDPSFLGRYMSAESLRPDTRYVLMDGRKVPTDNYITTYPGQCRFVTDTPKGPKVKYSTFKCYGVSPTKLNDKYEQYIKWPWYIWLGSSSKLWAQCVQLHNDYVGYLQPRTFLLKYN

SEQ ID NO: 37 - protein, 1712 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTRAKRSGSGATNFSLLKQAGDVEENPGPHSYFTSDYYQLYSTQKHYVYIGDPAQLPAPYALVYFLQSINFVRIATYYLFDESGEFKLASHKNPLLYDANYFLCWHRKVPTDNYITTYPGQITVATSRTLSYYKLGAPNMMVTNNTFTLKGGASIIKTIQPRVEKKKLKYEQYIKWPWYIWLGKDLSPRWYFYYLGTGTYKNTCDGTTFTYASAVHAGTDLEGNFYGPFESLRPDTRYVLMDGQTACTDDNALAYYNTTSSSKLWAQCVQLHNDYASALWEIQQVVDADDAYKTFPPTEPKKDKSTFKCYGVSPTKLNDAPSASAFFGMSRIGMLALLLLDRLNQLESKSLEIPRRNVATLQAEYYHTTDPSFLGRYMSARDVDTDFVNEFYAYLCRFVTDTPKGPKVKYIWVATEGALNTPKDHIYVGYLQPRTFLLKYN

SEQ ID NO: 38 - protein, 546 aa, Artificial Sequence
  Description of Artificial Sequence Synthetic polypeptide
MLRVEAFEYYHTTDPSFLGRYMSALNHTKKWVLQQLRVESSSKLWAQCVQLHNDILLAKDTAPSASAFFGMSRIGMEVTPSGTWLTYTGAIKLDDKDPNFKDQVILLNKHIDAYKTFPPTEPKKDKSLEIPRRNVATLQAELNESLIDLQELGKYEQYIKWPWYIWLGFIAGLIAIVMVQTACTDDNALAYYNTTKGGRFVLALLSDLQDLKWARFPKSDGTGTIYTELEPPCRFVTDTPKGPKVKYSIIKTIQPRVEKKKLITVATSRTLSYYKLGASQRVAGDSGFAAYSRYRIGNYKLNTDHSSSSDNIALLVQSKLIEYTDFATSACVLAAECTIFKDASGKPVPYCYDTNVLEGSVAYESLRPDTRYVLMDGEKYCALAPNMMVTNNTFTLKGGAPTKVTFGGDGKMKDLSPRWYFYYLGTGPEAGLPYGANKDGIIWVATEGALNTPKDHIGTRNPYALVYFLQSINFVRIIMRLWLCWKCRSKNPLLYDANYFLCWHTYKNTCDGTTFTYASALWEIQQVVDADKHYVYIGDPAQLPAP

SEQ ID NO: 39 - protein, 1099 aa, Artificial Sequence
  Description of Artificial Sequence Synthetic polypeptide
MYASALWEIQQVVDADSSSKLWAQCVQLHNDKHYVYIGDPAQLPAPSLEIPRRNVATLQAERDVDTDFVNEFYAYLTYKNTCDGTTFTYASAPNMMVTNNTFTLKGGAATYYLFDESGEFKLASHYYHTTDPSFLGRYMSAESLRPDTRYVLMDGQTACTDDNALAYYNTTSIIKTIQPRVEKKKLCRFVTDTPKGPKVKYSTFKCYGVSPTKLNDRKVPTDNYITTYPGQVHAGTDLEGNFYGPFYVGYLQPRTFLLKYNGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGEKWESGVKDCVVLHSYFTSDYYQLYSTQLSTDTGVEHVTFFIYNKIVDEPEEHVQIHTIDGSSGVVNPVMEPIYDEPTTTTSVPLSSMGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFKQGEIKDATPLDFVRATATIPIQASLPFGWLIVGVALLAVFQSASKIITLKKRWQLALSKGVHFVCNLLLLFVTVYSHLLLVAAGLEAMSDNGPQNQRNAPRITFGGPSDSTGSNQNGERSGARSKQRRPQGLPNNTASWFTALTQHGKEGLKFPRGQGVPINTNSSPDDQIGYYRRATRRIRGLFARTRSMWSFNPETNILLNVPLHGTILTRPLLESELVIGAVILRGHLRIAGHHLGRCDIKDLPKEPFLYLYALVYFLQSINFVRIIMRLWLCWKCRSKNPLLYDANYFLCWHTNCYDYCIPYNSVTSSIVITSGDGTTSPISEHDYQIGGYTAPSASAFFGMSRIGMEVTPSGTWLTYTGAIKLDDKDPNFKDQVILLNKHIDAYKTFPPTEPKKDKKKKAYETQALPQRQKKQQTVTLLPAADLDDFSKQLQQSMSSADSTQAITVATSRTLSYYKLGASQRVAGDSGFAAYSRYRIGNYKLNTDHSSSSDNIALLVQLNESLIDLQELGKYEQYIKWPWYIWLGFIAGLIAIVMV

SEQ ID NO: 40 - protein, 1221 aa, Artificial Sequence
  Description of Artificial Sequence Synthetic polypeptide
MATYYLFDESGEFKLASHSKLIEYTDFATSACVLAAECTIFKDASGKPVPYCYDTNVLEGSVAYESLRPDTRYVLMDGRKVPTDNYITTYPGQQTACTDDNALAYYNTTKGGRFVLALLSDLQDLKWARFPKSDGTGTIYTELEPPCRFVTDTPKGPKVKYSTFKCYGVSPTKLNDRDVDTDFVNEFYAYLTYKNTCDGTTFTYASALWEIQQVVDADLRVEAFEYYHTTDPSFLGRYMSALNHTKKWKHYVYIGDPAQLPAPSLEIPRRNVATLQAEVLQQLRVESSSKLWAQCVQLHNDILLAKDTVHAGTDLEGNFYGPFSIIKTIQPRVEKKKLEKYCALAPNMMVTNNTFTLKGGAPTKVTFGYVGYLQPRTFLLKYNGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGEKWESGVKDCVVLHSYFTSDYYQLYSTQLSTDTGVEHVTFFIYNKIVDEPEEHVQIHTIDGSSGVVNPVMEPIYDEPTTTTSVPLSSMGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFKQGEIKDATPLDFVRATATIPIQASLPFGWLIVGVALLAVFQSASKIITLKKRWQLALSKGVHFVCNLLLLFVTVYSHLLLVAAGLEAMSDNGPQNQRNAPRITFGGPSDSTGSNQNGERSGARSKQRRPQGLPNNTASWFTALTQHGKEGLKFPRGQGVPINTNSSPDDQIGYYRRATRRIRGLFARTRSMWSFNPETNILLNVPLHGTILTRPLLESELVIGAVILRGHLRIAGHHLGRCDIKDLPKEPFLYLYALVYFLQSINFVRIIMRLWLCWKCRSKNPLLYDANYFLCWHTNCYDYCIPYNSVTSSIVITSGDGTTSPISEHDYQIGGYTAPSASAFFGMSRIGMEVTPSGTWLTYTGAIKLDDKDPNFKDQVILLNKHIDAYKTFPPTEPKKDKKKKAYETQALPQRQKKQQTVTLLPAADLDDFSKQLQQSMSSADSTQAITVATSRTLSYYKLGASQRVAGDSGFAAYSRYRIGNYKLNTDHSSSSDNIALLVQLNESLIDLQELGKYEQYIKWPWYIWLGFIAGLIAIVMV

SEQ ID NO: 41 - protein, 1800 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTRAKRSGSGATNFSLLKQAGDVEENPGPYALVYFLQSINFVRIIMRLWLCWKCRSKNPLLYDANYFLCWHGDGKMKDLSPRWYFYYLGTGPEAGLPYGANKDGIIWVATEGALNTPKDHIGTRNPEKYCALAPNMMVTNNTFTLKGGAPTKVTFGTYKNTCDGTTFTYASALWEIQQVVDADLNESLIDLQELGKYEQYIKWPWYIWLGFIAGLIAIVMVQTACTDDNALAYYNTTKGGRFVLALLSDLQDLKWARFPKSDGTGTIYTELEPPCRFVTDTPKGPKVKYLRVEAFEYYHTTDPSFLGRYMSALNHTKKWITVATSRTLSYYKLGASQRVAGDSGFAAYSRYRIGNYKLNTDHSSSSDNIALLVQVLQQLRVESSSKLWAQCVQLHNDILLAKDTAPSASAFFGMSRIGMEVTPSGTWLTYTGAIKLDDKDPNFKDQVILLNKHIDAYKTFPPTEPKKDKSKLIEYTDFATSACVLAAECTIFKDASGKPVPYCYDTNVLEGSVAYESLRPDTRYVLMDG

SEQ ID NO: 42 - protein, 7096 aa, Severe acute respiratory syndrome coronavirus 2 [sequence omitted]

SEQ ID NO: 43 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 44 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 45 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 46 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 47 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 48 - DNA, 870 bp, Artificial Sequence
  Description of Artificial Sequence Synthetic polynucleotide
atgtacgccagcgccctgtgggagatccagcaggtggtggacgctgatcacagctacttcacctctgattactaccagctttattctacacagaagcactacgtgtatatcggcgaccccgcccaactgcctgcccctgacgcctacaaaaccttcccccccacagaacccaaaaaggacaaaatctgggtcgccaccgagggcgccctgaacacccctaaagaccatatcatcaccgtggccacaagcagaaccctgagctattacaagctgggcgcttacgccctggtgtactttctgcagagcatcaacttcgtgcggatctccctggaaatccctaggcggaacgttgctaccctgcaggccgagcctaatatgatggtgaccaacaacaccttcaccctcaagggcggagccaagaaccccctgctgtacgacgccaattacttcctgtgctggcacaaggacctgtctccaagatggtatttctactacctgggcaccggctactaccacaccacagatccaagcttcctgggaagatacatgagcgctgaaagcctgagacctgataccagatacgtgctgatggacggcagaaaggtgcctaccgacaactacattaccacataccccggccagtgtagattcgtgacagacacccctaagggacctaaggtgaagtacagcacattcaagtgctacggcgtgtccccaacaaagctgaatgataaatacgagcagtacatcaagtggccttggtacatctggctgggcagcagctccaagctgtgggcccagtgcgtgcagctgcacaacgactacgtcggctacctgcaacctcggacatttctgctgaagtacaactag

SEQ ID NO: 49 - DNA, 690 bp, Artificial Sequence
  Description of Artificial Sequence Synthetic polynucleotide
atggccctgtgggagatccagcaggttgtgaaggctgcattcacctctgattactaccagctgtacaaggccgccgtgtacatcggcgaccccgcccagctgaaggccgctaaaaccttcccccccaccgagcctaagaaagctgctgcaacagaaggcgccctgaatacacctaagaaagccgccgccacaagcagaacactgagctactacaagaaggccgctgtgtatttcctccagagcatcaacttcaaggccgctatccccaggcggaacgtggccaccctgaaagctgccatggtcaccaacaacacctttaccctgaagaaggccgccctgctgtacgacgccaattactttctgaaggccgcatctccacggtggtacttctactacctgaaggccgccaccaccgaccctagcttcctgggcagatacaaggccgccagacctgacaccagatacgtgctgaaagccgcccctaccgacaactacattacaacctacaaggccgccgtgacagatacccctaagggacctaaaaaggccgccaagtgctacggcgtgtccccaacaaagaaggccgctcagtacatcaagtggccttggtatatcaaggccgccaagctgtgggcccaatgtgtgcagctgaaggccgcctatctgcagcctagaaccttcctgctgtag

SEQ ID NO: 50 - DNA, 528 bp, Artificial Sequence
  Description of Artificial Sequence Synthetic polynucleotide
atggccctgtgggagatccagcaggtggtgttcaccagcgactactaccagctgtacgtgtacatcggcgaccccgctcagctgaagaccttcccacctaccgaacccaaggccaccgagggagccctgaacacccctaaggctacatctagaaccctgagctactacaaggtgtacttcctgcagtctatcaacttcatcccccggagaaacgtggccaccctgatggttaccaacaacacctttaccctgaagctgctgtacgacgccaattacttcctcagccctcggtggtatttctactaccttacaaccgaccctagcttcctgggcagatacagacctgacacaagatatgtgctgcctacagataattacatcaccacatacgtcaccgatacccccaaaggccctaaaaagtgctacggcgtgtcccctacaaagcagtacattaagtggccttggtacatcaagctgtgggcccagtgtgtgcagctgtatctgcaaccacggacatttctgctgtag

SEQ ID NO: 51 - DNA, 1248 bp, Artificial Sequence
  Description of Artificial Sequence Synthetic polynucleotide
atgcacagctacttcaccagcgactactaccagctgtacagcacccagaagcactacgtgtacatcggcgaccccgcccagctgcccgccccctacgccctggtgtacttcctgcagagcatcaacttcgtgaggatcgccacctactacctgttcgacgagagcggcgagttcaagctggccagccacaagaaccccctgctgtacgacgccaactacttcctgtgctggcacaggaaggtgcccaccgacaactacatcaccacctaccccggccagatcaccgtggccaccagcaggaccctgagctactacaagctgggcgcccccaacatgatggtgaccaacaacaccttcaccctgaagggcggcgccagcatcatcaagaccatccagcccagggtggagaagaagaagctgaagtacgagcagtacatcaagtggccctggtacatctggctgggcaaggacctgagccccaggtggtacttctactacctgggcaccggcacctacaagaacacctgcgacggcaccaccttcacctacgccagcgccgtgcacgccggcaccgacctggagggcaacttctacggccccttcgagagcctgaggcccgacaccaggtacgtgctgatggacggccagaccgcctgcaccgacgacaacgccctggcctactacaacaccaccagcagcagcaagctgtgggcccagtgcgtgcagctgcacaacgactacgccagcgccctgtgggagatccagcaggtggtggacgccgacgacgcctacaagaccttcccccccaccgagcccaagaaggacaagagcaccttcaagtgctacggcgtgagccccaccaagctgaacgacgcccccagcgccagcgccttcttcggcatgagcaggatcggcatgctggccctgctgctgctggacaggctgaaccagctggagagcaagagcctggagatccccaggaggaacgtggccaccctgcaggccgagtactaccacaccaccgaccccagcttcctgggcaggtacatgagcgccagggacgtggacaccgacttcgtgaacgagttctacgcctacctgtgcaggttcgtgaccgacacccccaagggccccaaggtgaagtacatctgggtggccaccgagggcgccctgaacacccccaaggaccacatctacgtgggctacctgcagcccaggaccttcctgctgaagtacaactgataatag

SEQ ID NO: 52 - DNA, 990 bp, Artificial Sequence
  Description of Artificial Sequence Synthetic polynucleotide
atgtttacgtcagactactatcagctttacaaagcagccgtctatatcggggatcccgcccagcttaaagccgccgtgtatttcctgcagtccatcaacttcaaagcagcgtatttgttcgatgagtctggggagtttaaattgaaagctgccctcctctacgacgccaattattttcttaaagcggccccaacggataactatataacgacctacaaagcagctgccacatcccgaacactcagttattataagaaagcagccatggttacgaataacacttttacgctgaaaaaagctgcaaaaacgattcagccgcgggttgaaaagaaagctgcgcagtacataaagtggccatggtatatcaaagccgcctcccccagatggtacttctattatttgaaagcggccaacacatgcgacggtaccacgtttacatataaagctgcggggacagacttggaagggaatttttacaaggcggctcgcccagatacacgctacgttttgaaagccgcttgtactgatgataacgcattggcttactataaagcagcaaagctttgggcccagtgcgttcagctgaaggccgcagcgctctgggaaatccaacaggttgtgaaagccgcaaaaactttcccgccgacagaaccgaaaaaggcggcgaagtgctatggagtcagtcctactaaaaaggccgccgcgtcagccttctttggcatgagtcgcaaggctgctctgcttttggatcggctcaatcaactcaaagccgcaataccgagaaggaacgttgcgacattgaaggcggccacaacagacccgtcattcctgggcagatacaaggcagctgataccgacttcgtcaatgagttttacaaggcggcggtcactgacacaccgaaaggccccaaaaaggccgctgcgaccgagggtgcgttgaatacaccaaaaaaggcagcatatctccagccaaggacgttcctgctgtag

SEQ ID NO: 53 - DNA, 756 bp, Artificial Sequence
  Description of Artificial Sequence Synthetic polynucleotide
atgttcacctctgattactaccagctttatgtgtacattggagatcctgctcagctggtgtacttcctgcagagcatcaacttctacctgttcgacgagagcggcgaattcaagctgctgctgtacgacgccaactacttcctgcctaccgataattacatcaccacatacgccacatctcggaccctgagctactacaagatggtcaccaacaacacctttaccctgaagaagaccatccagcctcgggtggaaaagcagtacatcaagtggccttggtatatcagccctagatggtacttttactacctgaacacctgcgacggcaccacctttacatacggcacagatctggaaggcaacttctacagacctgacaccagatacgtgctgtgcaccgacgacaatgccctggcctactataagctgtgggcccaatgtgtgcagctggctctgtgggagatccagcaggtggtgaagacattcccccccaccgagcccaagaaatgctacggcgtgtccccaacaaaggccagcgccttcttcggcatgagccggctgctgctggacagactgaaccagctgatcccccgcaggaacgtggccaccctgaccaccgacccctccttcctgggaagatacgacacagacttcgtgaacgagttctacgttacagatacaccaaaaggccctaaagctacagagggcgccctgaatacccctaagtacctgcaacctagaacctttctgctctag

SEQ ID NO: 54 - DNA, 1506 bp, Artificial Sequence
  Description of Artificial Sequence Synthetic polynucleotide
atgtacgctctggtttattttcttcagtccatcaacttcgtaaggatcattatgcgactctggctgtgctggaagtgtcgcagcaagaaccccctcttgtatgacgctaattacttcctgtgctggcacggggacggcaagatgaaagatctgagccccagatggtacttctactatcttggtaccggacccgaagccgggctgccatacggggcaaataaagacggtattatctgggttgccactgagggtgccctgaatactcctaaagaccacattggcacacggaatcccgagaagtactgcgcattggctcccaacatgatggtgaccaacaatacctttacattaaaggggggagcaccaaccaaagtgacttttggcacttacaagaacacatgtgacggcacgacttttacctacgcaagcgctctgtgggaaattcaacaggtcgtcgacgccgatcttaacgagtctcttatcgatttacaggaattgggtaaatatgaacagtatatcaagtggccttggtacatctggctaggctttatagcaggacttatagctatcgtcatggttcagaccgcttgtacagacgataatgccctcgcctattataataccaccaaaggtgggcgttttgtgctggccctgctgtccgatctgcaggatctgaagtgggctcgcttcccaaaaagtgacggaaccggcacgatctacaccgagttggagccaccttgtcgcttcgtgaccgatacgccaaaaggccctaaagtgaagtatctccgggttgaagcattcgaatactatcataccacagatccttccttcctcgggaggtacatgagtgccctaaatcatactaagaaatggattaccgtggccacttcacgaactctctcctactacaaactgggcgcttcacagcgggtagccggcgacagcggattcgccgcctactccaggtatagaattggaaactataagctgaacacagaccattcttctagcagcgacaacatcgctctgttggtgcaggtcttgcaacagctgcgggtggagtcttcaagtaagttatgggcacaatgcgttcaacttcacaatgatattctactggcgaaagacacggccccctctgcgtccgccttttttggaatgtctagaatagggatggaagtaacaccatccggcacatggctcacatatacaggcgcgatcaagttagacgacaaggacccgaatttcaaagatcaggtgattctgctcaacaagcacatcgatgcatataagacatttccacctacggagcccaagaaggacaaaagcaagctcatagagtacactgatttcgcaacctcggcttgtgtcctcgctgccgagtgcaccattttcaaagatgcctcagggaagcctgtgccgtattgctacgatacaaacgtgcttgaaggttcggtggcgtacgagagtctgaggcccgatactagatatgtcctgatggacggatag

SEQ ID NO: 55 - DNA, 4767 bp, Artificial Sequence
  Description of Artificial Sequence Synthetic polynucleotide
atgttcgtgttcctggtgctgctccccctggtctctagccagtgcgtgaatttaaccacacgcacccaactgccccccgcatacaccaactcctttaccagaggcgtgtactaccctgataaggtgtttagatctagcgttttgcacagcacacaggacctcttcctgccattcttcagcaatgtgacctggttccacgccattcacgtgtccggcacaaacggcactaagagattcgacaaccccgtgctgccttttaatgacggcgtgtactttgcatccaccgagaagtctaacatcatcagaggctggatattcggcaccaccctcgatagcaagacacagagcctgctcatcgtgaacaacgccacaaacgtggtgatcaaggtgtgcgagtttcagttctgcaacgaccctttcctgggggtctactaccacaaaaacaacaagagctggatggaatctgagttccgggtgtactccagcgccaataattgcaccttcgagtacgtgtcccagcccttcctgatggacttggagggcaagcagggcaattttaagaacctgagagagttcgtgtttaaaaatatcgacggctacttcaagatctacagcaagcatacacctatcaatctggtgagagacctgcctcagggcttcagcgcattggagcccctggttgacctgcccatcggcatcaacatcacaagattccagacactgctggccctgcacagaagctacctgacccctggagattcctcttctggatggaccgccggagccgccgcctactacgtgggatacctgcagcctagaaccttcctactgaagtacaatgagaatggaaccatcaccgatgccgtggactgcgctctggaccccctgagcgagacaaagtgtaccctgaagagcttcaccgtggaaaagggcatctatcagaccagcaacttccgagtccagcctacagagagcattgtgcggttccctaacatcacaaacctgtgcccttttggggaagtgttcaacgcgacccggttcgccagcgtgtatgcctggaacagaaaacggatctccaactgcgtggcagattacagcgtgctgtacaacagcgccagtttcagcaccttcaagtgctacggcgtgagccctacaaaactgaacgatctgtgcttcaccaatgtgtacgccgactctttcgtgatcaggggcgacgaagtgaggcagattgcccccggccagacgggcaaaatcgccgactacaactacaagctgcccgacgacttcaccggctgcgttattgcctggaactctaacaacctcgattctaaagtgggcggaaactacaactacctgtaccggctgttcagaaaaagcaacctgaaacctttcgagagagacatatccacagaaatctaccaggctggctctactccttgtaatggcgttgaagggttcaactgctactttccactgcagagctacggctttcagcctacaaacggagtgggctaccagccctaccgtgtggtggtgctgagcttcgaactgttgcacgcccccgctaccgtctgcggcccgaagaagagcacgaacctggtaaagaacaagtgcgtgaatttcaacttcaacggcctgacaggcaccggcgtgctgaccgaatcgaacaagaagtttctgccatttcagcagttcggccgggacatcgccgacaccaccgatgccgtgcgggatcctcagacactagaaatcctggacatcaccccctgtagcttcggcggcgtgagcgtcatcacacctggcacaaacaccagcaaccaggtggccgtgctctaccaggacgtgaattgcacagaggtgcctgtggccatccacgccgatcagctgacccctacatggcgggtctactctacaggaagcaacgtgttccagacaagagccggatgccttataggcgccgagcacgtgaacaacagctacgaatgcgacatccctatcggcgccggcatctgtgcttcttaccagacacagactaactctccacggagagccagatcagtggcctctcagtctatcatcgcctataccatgagtctgggagccgagaacagcgtggcatacagcaacaacagcatcgccatcccaaccaacttcacaatcagtgtgacaaccgagatcctgcccgtgtccatgaccaagacctcggtggattgcacgatgtacatctgcggcgacagcaccgaatgcagcaacctcctgctccagtacggcagtttctgcacacagctgaaccgggccctaaccggcatcgccgtggaacaggacaaaaacacccaggaggtgttcgcccaagtgaagcaaatctacaagaccccacctatcaaggacttcggcggcttcaattttagccagatcctgccagacccttctaagccttcaaagagaagcttcatcgaggacctgttattcaacaaagtgacgctggccgatgctggcttcatcaagcaatacggcgactgcctcggcgacatcgcggcacgagacctgatctgcgcccagaagtttaacggactgaccgtgttaccacctcttctgaccgatgaaatgatcgcacagtacaccagcgccctcctggccggcaccatcaccagcggatggaccttcggagcgggcgctgcactgcagatccctttcgccatgcagatggcctacagatttaatgggattggcgtgacccaaaacgtgctgtacgaaaaccagaaattgatcgctaaccaattcaactctgccattggaaagatccaggatagcctgagctctaccgcatccgctctgggcaagttgcaagacgtggtgaaccagaacgcccaggccctgaacacgctggtgaaacagctgagcagcaacttcggcgctattagcagcgtgctgaatgatattctgtcccggctggacccgcctgaggctgaggtgcagattgatcggctgattacgggtcggctgcagagcctgcagacctacgttacccagcagttgatcagagccgccgagatcagagccagtgcgaacctggcggcaaccaagatgagcgagtgtgtcctcggacagagcaagcgggttgacttctgtggaaagggctaccacctgatgagcttcccccagtctgccccgcacggcgtggtgttcctgcacgtgacctatgtgccagcccaggagaaaaatttcaccactgcccctgctatctgtcatgatggcaaggcccacttccccagagaaggcgttttcgtgagcaatggcacacactggttcgtgacacagcggaacttttacgagcctcagatcatcacaactgacaatactttcgtgagtggcaactgcgacgtcgtcatcggcatcgtgaacaacaccgtgtacgaccctctgcaacctgagctggacagcttcaaggaagagctggataagtacttcaagaaccacaccagccccgacgtagatctgggcgacatcagcggcatcaacgccagcgtggtcaacatccagaaggagatcgacagactgaacgaggttgccaagaatctgaacgaaagcctgatcgacctgcaggaactgggcaagtacgagcagtacatcaaatggccttggtacatctggctgggcttcatcgccggcctgatcgccatcgtgatggtcaccatcatgctgtgttgcatgacctcatgctgctcttgcctgaagggctgttgtagctgcggctcttgttgcaagttcgacgaagatgacagcgagcctgtgctcaagggcgtgaagctgcactacacccgggccaagcggagcggctccggcgccaccaattttagcctgctaaagcaggccggcgacgtcgaggagaaccccggcccttatgccagcgctctgtgggagatccagcaggtggtggacgccgaccacagctacttcaccagcgactactaccagctgtactctacccaaaagcactacgtgtacatcggcgatccagcccagctgccagcccctgatgcttacaagactttccccccaacagaacctaagaaagataaaatttgggtggccactgaaggcgccctgaacacgcctaaggaccacatcatcacagtggccaccagcagaacactgagctactacaagttaggcgcctacgctttggtgtatttcctgcagagcatcaacttcgtgcgcatcagcctggaaatccccagaaggaacgtggccactctgcaggctgagcctaacatgatggtgaccaacaacacctttaccctgaagggcggagccaagaaccccctgctgtacgatgctaattacttcctgtgctggcacaaggatctgagtcctagatggtacttttactacctgggcacgggttactaccacaccaccgaccccagcttcctgggcagatacatgtccgcagaaagcctgagacctgataccagatacgtgctgatggacggcagaaaggtgcctacagacaactacatcaccacctaccctggccagtgcagattcgtaacggacacacctaagggccccaaggtgaagtacagcacttttaagtgctacggagtgtctcctacaaagctgaacgataagtacgagcagtacataaagtggccctggtacatttggctgggcagcagtagcaagctatgggcccagtgtgttcagctgcataacgactacgtgggatacctgcagccaagaacctttctgctgaagtacaactag

SEQ ID NO: 56 - DNA, 5139 bp, Artificial Sequence
  Description of Artificial Sequence Synthetic polynucleotide
atgttcgtgttcctggtgctgctccccctggtctctagccagtgcgtgaatttaaccacacgcacccaactgccccccgcatacaccaactcctttaccagaggcgtgtactaccctgataaggtgtttagatctagcgttttgcacagcacacaggacctcttcctgccattcttcagcaatgtgacctggttccacgccattcacgtgtccggcacaaacggcactaagagattcgacaaccccgtgctgccttttaatgacggcgtgtactttgcatccaccgagaagtctaacatcatcagaggctggatattcggcaccaccctcgatagcaagacacagagcctgctcatcgtgaacaacgccacaaacgtggtgatcaaggtgtgcgagtttcagttctgcaacgaccctttcctgggggtctactaccacaaaaacaacaagagctggatggaatctgagttccgggtgtactccagcgccaataattgcaccttcgagtacgtgtcccagcccttcctgatggacttggagggcaagcagggcaattttaagaacctgagagagttcgtgtttaaaaatatcgacggctacttcaagatctacagcaagcatacacctatcaatctggtgagagacctgcctcagggcttcagcgcattggagcccctggttgacctgcccatcggcatcaacatcacaagattccagacactgctggccctgcacagaagctacctgacccctggagattcctcttctggatggaccgccggagccgccgcctactacgtgggatacctgcagcctagaaccttcctactgaagtacaatgagaatggaaccatcaccgatgccgtggactgcgctctggaccccctgagcgagacaaagtgtaccctgaagagcttcaccgtggaaaagggcatctatcagaccagcaacttccgagtccagcctacagagagcattgtgcggttccctaacatcacaaacctgtgcccttttggggaagtgttcaacgcgacccggttcgccagcgtgtatgcctggaacagaaaacggatctccaactgcgtggcagattacagcgtgctgtacaacagcgccagtttcagcaccttcaagtgctacggcgtgagccctacaaaactgaacgatctgtgcttcaccaatgtgtacgccgactctttcgtgatcaggggcgacgaagtgaggcagattgcccccggccagacgggcaaaatcgccgactacaactacaagctgcccgacgacttcaccggctgcgttattgcctggaactctaacaacctcgattctaaagtgggcggaaactacaactacctgtaccggctgttcagaaaaagcaacctgaaacctttcgagagagacatatccacagaaatctaccaggctggctctactccttgtaatggcgttgaagggttcaactgctactttccactgcagagctacggctttcagcctacaaacggagtgggctaccagccctaccgtgtggtggtgctgagcttcgaactgttgcacgcccccgctaccgtctgcggcccgaagaagagcacgaacctggtaaagaacaagtgcgtgaatttcaacttcaacggcctgacaggcaccggcgtgctgaccgaatcgaacaagaagtttctgccatttcagcagttcggccgggacatcgccgacaccaccgatgccgtgcgggatcctcagacactagaaatcctggacatcaccccctgtagcttcggcggcgtgagcgtcatcacacctggcacaaacaccagcaaccaggtggccgtgctctaccaggacgtgaattgcacagaggtgcctgtggccatccacgccgatcagctgacccctacatggcgggtctactctacaggaagcaacgtgttccagacaagagccggatgccttataggcgccgagcacgtgaacaacagctacgaatgcgacatccctatcggcgccggcatctgtgcttcttaccagacacagactaactctccacggagagccagatcagtggcctctcagtctatcatcgcctataccatgagtctgggagccgagaacagcgtggcatacagcaacaacagcatcgccatcccaaccaacttcacaatcagtgtgacaaccgagatcctgcccgtgtccatgaccaagacctcggtggattgcacgatgtacatctgcggcgacagcaccgaatgcagcaacctcctgctccagtacggcagtttctgcacacagctgaaccgggccctaaccggcatcgccgtggaacaggacaaaaacacccaggaggtgttcgcccaagtgaagcaaatctacaagaccccacctatcaaggacttcggcggcttcaattttagccagatcctgccagacccttctaagccttcaaagagaagcttcatcgaggacctgttattcaacaaagtgacgctggccgatgctggcttcatcaagcaatacggcgactgcctcggcgacatcgcggcacgagacctgatctgcgcccagaagtttaacggactgaccgtgttaccacctcttctgaccgatgaaatgatcgcacagtacaccagcgccctcctggccggcaccatcaccagcggatggaccttcggagcgggcgctgcactgcagatccctttcgccatgcagatggcctacagatttaatgggattggcgtgacccaaaacgtgctgtacgaaaaccagaaattgatcgctaaccaattcaactctgccattggaaagatccaggatagcctgagctctaccgcatccgctctgggcaagttgcaagacgtggtgaaccagaacgcccaggccctgaacacgctggtgaaacagctgagcagcaacttcggcgctattagcagcgtgctgaatgatattctgtcccggctggacccgcctgaggctgaggtgcagattgatcggctgattacgggtcggctgcagagcctgcagacctacgttacccagcagttgatcagagccgccgagatcagagccagtgcgaacctggcggcaaccaagatgagcgagtgtgtcctcggacagagcaagcgggttgacttctgtggaaagggctaccacctgatgagcttcccccagtctgccccgcacggcgtggtgttcctgcacgtgacctatgtgccagcccaggagaaaaatttcaccactgcccctgctatctgtcatgatggcaaggcccacttccccagagaaggcgttttcgtgagcaatggcacacactggttcgtgacacagcggaacttttacgagcctcagatcatcacaactgacaatactttcgtgagtggcaactgcgacgtcgtcatcggcatcgtgaacaacaccgtgtacgaccctctgcaacctgagctggacagcttcaaggaagagctggataagtacttcaagaaccacaccagccccgacgtagatctgggcgacatcagcggcatcaacgccagcgtggtcaacatccagaaggagatcgacagactgaacgaggttgccaagaatctgaacgaaagcctgatcgacctgcaggaactgggcaagtacgagcagtacatcaaatggccttggtacatctggctgggcttcatcgccggcctgatcgccatcgtgatggtcaccatcatgctgtgttgcatgacctcatgctgctcttgcctgaagggctgttgtagctgcggctcttgttgcaagttcgacgaagatgacagcgagcctgtgctcaagggcgtgaagctgcactacacccgggccaagcggagcggctccggcgccaccaattttagcctgctaaagcaggccggcgacgtcgaggagaaccccggccctcactcctactttacaagtgattactaccaactgtactcaacacagaagcactacgtgtacatcggcgatcctgcccagctgccagccccttacgccctagtgtattttctgcaatctatcaacttcgtccggattgccacgtactatctgttcgacgagtctggcgaattcaaactggcctcccacaagaaccctctgctgtatgacgctaactacttcctatgctggcacagaaaggtgcccaccgataactacatcacaacctaccctggccagataaccgtggccacaagcagaacactgtcctactacaagctgggtgctcctaatatgatggtgaccaacaacacattcaccctgaagggcggagccagtatcatcaagaccatccagcctagagtggaaaagaaaaagctgaagtacgagcagtacatcaagtggccttggtatatctggctgggaaaagacctgagccccagatggtacttttactatctgggcacaggcacctacaagaacacctgtgacggcacaacattcacctacgcctctgccgtgcacgccggaacagacctggaaggcaacttctacggacctttcgagagcctgagacccgataccaggtacgtgctgatggatggccagaccgcctgtacagacgacaatgctctggcctactataacaccactagcagctcaaagctgtgggcccagtgcgtgcagctgcataacgattacgccagcgccctgtgggagatccaacaggtggtggacgccgacgacgcttataagaccttccctcccaccgagcccaagaaggacaagagtaccttcaaatgctacggcgtttctcccaccaaactgaacgacgctcctagcgccagtgccttctttggaatgtcaagaatcggcatgcttgccctgctgctgctcgatagactgaaccagctggaatccaagagccttgagatccccagaagaaacgtggctaccctgcaggccgagtactaccacaccaccgacccttccttcctggggagatacatgagcgccagagacgtcgatactgacttcgtaaatgagttctacgcctacctgtgtagattcgtcacagacaccccaaagggccctaaggtcaagtacatttgggtggccaccgaaggcgctctgaacactcctaaggatcacatctatgtcggctacctgcagcctcggacattcctgctgaaatacaactag

SEQ ID NO: 57 - DNA, 1647 bp, Artificial Sequence
  Description of Artificial Sequence Synthetic polynucleotide
atgctgagggtggaggccttcgagtactaccacaccaccgaccccagcttcctgggcaggtacatgagcgccctgaaccacaccaagaagtgggtgctgcagcagctgagggtggagagcagcagcaagctgtgggcccagtgcgtgcagctgcacaacgacatcctgctggccaaggacaccgcccccagcgccagcgccttcttcggcatgagcaggatcggcatggaggtgacccccagcggcacctggctgacctacaccggcgccatcaagctggacgacaaggaccccaacttcaaggaccaggtgatcctgctgaacaagcacatcgacgcctacaagaccttcccccccaccgagcccaagaaggacaagagcctggagatccccaggaggaacgtggccaccctgcaggccgagctgaacgagagcctgatcgacctgcaggagctgggcaagtacgagcagtacatcaagtggccctggtacatctggctgggcttcatcgccggcctgatcgccatcgtgatggtgcagaccgcctgcaccgacgacaacgccctggcctactacaacaccaccaagggcggcaggttcgtgctggccctgctgagcgacctgcaggacctgaagtgggccaggttccccaagagcgacggcaccggcaccatctacaccgagctggagcccccctgcaggttcgtgaccgacacccccaagggccccaaggtgaagtacagcatcatcaagaccatccagcccagggtggagaagaagaagctgatcaccgtggccaccagcaggaccctgagctactacaagctgggcgccagccagagggtggccggcgacagcggcttcgccgcctacagcaggtacaggatcggcaactacaagctgaacaccgaccacagcagcagcagcgacaacatcgccctgctggtgcagagcaagctgatcgagtacaccgacttcgccaccagcgcctgcgtgctggccgccgagtgcaccatcttcaaggacgccagcggcaagcccgtgccctactgctacgacaccaacgtgctggagggcagcgtggcctacgagagcctgaggcccgacaccaggtacgtgctgatggacggcgagaagtactgcgccctggcccccaacatgatggtgaccaacaacaccttcaccctgaagggcggcgcccccaccaaggtgaccttcggcggcgacggcaagatgaaggacctgagccccaggtggtacttctactacctgggcaccggccccgaggccggcctgccctacggcgccaacaaggacggcatcatctgggtggccaccgagggcgccctgaacacccccaaggaccacatcggcaccaggaacccctacgccctggtgtacttcctgcagagcatcaacttcgtgaggatcatcatgaggctgtggctgtgctggaagtgcaggagcaagaaccccctgctgtacgacgccaactacttcctgtgctggcacacctacaagaacacctgcgacggcaccaccttcacctacgccagcgccctgtgggagatccagcaggtggtggacgccgacaagcactacgtgtacatcggcgaccccgcccagctgcccgccccctgataatag

SEQ ID NO: 58 - DNA, 3306 bp, Artificial Sequence
  Description of Artificial Sequence Synthetic polynucleotide
atgtacgccagcgccctgtgggagatccagcaggtggtggacgccgacagcagcagcaagctgtgggcccagtgcgtgcagctgcacaacgacaagcactacgtgtacatcggcgaccccgcccagctgcccgcccccagcctggagatccccagaagaaacgtggccaccctgcaggccgagagagacgtggacaccgacttcgtgaacgagttctacgcctacctgacctacaagaacacctgcgacggcaccaccttcacctacgccagcgcccccaacatgatggtgaccaacaacaccttcaccctgaagggcggcgccgccacctactacctgttcgacgagagcggcgagttcaagctggccagccactactaccacaccaccgaccccagcttcctgggcagatacatgagcgccgagagcctgagacccgacaccagatacgtgctgatggacggccagaccgcctgcaccgacgacaacgccctggcctactacaacaccaccagcatcatcaagaccatccagcccagagtggagaagaagaagctgtgcagattcgtgaccgacacccccaagggccccaaggtgaagtacagcaccttcaagtgctacggcgtgagccccaccaagctgaacgacagaaaggtgcccaccgacaactacatcaccacctaccccggccaggtgcacgccggcaccgacctggagggcaacttctacggccccttctacgtgggctacctgcagcccagaaccttcctgctgaagtacaacggcgacggcaagatgaaggacctgagccccagatggtacttctactacctgggcaccggccccgaggccggcctgccctacggcgccaacaaggacggcatcatctgggtggccaccgagggcgccctgaacacccccaaggaccacatcggcaccagaaaccccgccaacaacgccgccatcgtgctgcagctgccccagggcaccaccctgcccaagggcttctacgccgagggcagcagaggcggcagccaggccagcagcagaagcagcagcagaagcagaaacagcagcagaaacagcacccccggcgagaagtgggagagcggcgtgaaggactgcgtggtgctgcacagctacttcaccagcgactactaccagctgtacagcacccagctgagcaccgacaccggcgtggagcacgtgaccttcttcatctacaacaagatcgtggacgagcccgaggagcacgtgcagatccacaccatcgacggcagcagcggcgtggtgaaccccgtgatggagcccatctacgacgagcccaccaccaccaccagcgtgcccctgagcagcatgggcaccagccccgccagaatggccggcaacggcggcgacgccgccctggccctgctgctgctggacagactgaaccagctggagagcaagatgagcggcaagggccagcagcagcagggccagaccgtgaccaagaagagcgccgccgaggccagcaagaagcccagacagaagagaaccgccaccaaggcctacaacgtgacccaggccttcggcagaagaggccccgagcagacccagggcaacttcggcgaccaggagctgatcagacagggcaccgactacaagcactggccccagatcgcccagttcaagcagggcgagatcaaggacgccacccccctggacttcgtgagagccaccgccaccatccccatccaggccagcctgcccttcggctggctgatcgtgggcgtggccctgctggccgtgttccagagcgccagcaagatcatcaccctgaagaagagatggcagctggccctgagcaagggcgtgcacttcgtgtgcaacctgctgctgctgttcgtgaccgtgtacagccacctgctgctggtggccgccggcctggaggccatgagcgacaacggcccccagaaccagagaaacgcccccagaatcaccttcggcggccccagcgacagcaccggcagcaaccagaacggcgagagaagcggcgccagaagcaagcagagaagaccccagggcctgcccaacaacaccgccagctggttcaccgccctgacccagcacggcaaggagggcctgaagttccccagaggccagggcgtgcccatcaacaccaacagcagccccgacgaccagatcggctactacagaagagccaccagaagaatcagaggcctgttcgccagaaccagaagcatgtggagcttcaaccccgagaccaacatcctgctgaacgtgcccctgcacggcaccatcctgaccagacccctgctggagagcgagctggtgatcggcgccgtgatcctgagaggccacctgagaatcgccggccaccacctgggcagatgcgacatcaaggacctgcccaaggagcccttcctgtacctgtacgccctggtgtacttcctgcagagcatcaacttcgtgagaatcatcatgagactgtggctgtgctggaagtgcagaagcaagaaccccctgctgtacgacgccaactacttcctgtgctggcacaccaactgctacgactactgcatcccctacaacagcgtgaccagcagcatcgtgatcaccagcggcgacggcaccaccagccccatcagcgagcacgactaccagatcggcggctacaccgcccccagcgccagcgccttcttcggcatgagcagaatcggcatggaggtgacccccagcggcacctggctgacctacaccggcgccatcaagctggacgacaaggaccccaacttcaaggaccaggtgatcctgctgaacaagcacatcgacgcctacaagaccttcccccccaccgagcccaagaaggacaagaagaagaaggcctacgagacccaggccctgccccagagacagaagaagcagcagaccgtgaccctgctgcccgccgccgacctggacgacttcagcaagcagctgcagcagagcatgagcagcgccgacagcacccaggccatcaccgtggccaccagcagaaccctgagctactacaagctgggcgccagccagagagtggccggcgacagcggcttcgccgcctacagcagatacagaatcggcaactacaagctgaacaccgaccacagcagcagcagcgacaacatcgccctgctggtgcagctgaacgagagcctgatcgacctgcaggagctgggcaagtacgagcagtacatcaagtggccctggtacatctggctgggcttcatcgccggcctgatcgccatcgtgatggtgtgataatag

SEQ ID NO: 59 - DNA, 3672 bp, Artificial Sequence
  Description of Artificial Sequence Synthetic polynucleotide
atggccacctactacctgttcgacgagagcggcgagttcaagctggccagccacagcaagctgatcgagtacaccgacttcgccaccagcgcctgcgtgctggccgccgagtgcaccatcttcaaggacgccagcggcaagcccgtgccctactgctacgacaccaacgtgctggagggcagcgtggcctacgagagcctgagacccgacaccagatacgtgctgatggacggcagaaaggtgcccaccgacaactacatcaccacctaccccggccagcagaccgcctgcaccgacgacaacgccctggcctactacaacaccaccaagggcggcagattcgtgctggccctgctgagcgacctgcaggacctgaagtgggccagattccccaagagcgacggcaccggcaccatctacaccgagctggagcccccctgcagattcgtgaccgacacccccaagggccccaaggtgaagtacagcaccttcaagtgctacggcgtgagccccaccaagctgaacgacagagacgtggacaccgacttcgtgaacgagttctacgcctacctgacctacaagaacacctgcgacggcaccaccttcacctacgccagcgccctgtgggagatccagcaggtggtggacgccgacctgagagtggaggccttcgagtactaccacaccaccgaccccagcttcctgggcagatacatgagcgccctgaaccacaccaagaagtggaagcactacgtgtacatcggcgaccccgcccagctgcccgcccccagcctggagatccccagaagaaacgtggccaccctgcaggccgaggtgctgcagcagctgagagtggagagcagcagcaagctgtgggcccagtgcgtgcagctgcacaacgacatcctgctggccaaggacaccgtgcacgccggcaccgacctggagggcaacttctacggccccttcagcatcatcaagaccatccagcccagagtggagaagaagaagctggagaagtactgcgccctggcccccaacatgatggtgaccaacaacaccttcaccctgaagggcggcgcccccaccaaggtgaccttcggctacgtgggctacctgcagcccagaaccttcctgctgaagtacaacggcgacggcaagatgaaggacctgagccccagatggtacttctactacctgggcaccggccccgaggccggcctgccctacggcgccaacaaggacggcatcatctgggtggccaccgagggcgccctgaacacccccaaggaccacatcggcaccagaaaccccgccaacaacgccgccatcgtgctgcagctgccccagggcaccaccctgcccaagggcttctacgccgagggcagcagaggcggcagccaggccagcagcagaagcagcagcagaagcagaaacagcagcagaaacagcacccccggcgagaagtgggagagcggcgtgaaggactgcgtggtgctgcacagctacttcaccagcgactactaccagctgtacagcacccagctgagcaccgacaccggcgtggagcacgtgaccttcttcatctacaacaagatcgtggacgagcccgaggagcacgtgcagatccacaccatcgacggcagcagcggcgtggtgaaccccgtgatggagcccatctacgacgagcccaccaccaccaccagcgtgcccctgagcagcatgggcaccagccccgccagaatggccggcaacggcggcgacgccgccctggccctgctgctgctggacagactgaaccagctggagagcaagatgagcggcaagggccagcagcagcagggccagaccgtgaccaagaagagcgccgccgaggccagcaagaagcccagacagaagagaaccgccaccaaggcctacaacgtgacccaggccttcggcagaagaggccccgagcagacccagggcaacttcggcgaccaggagctgatcagacagggcaccgactacaagcactggccccagatcgcccagttcaagcagggcgagatcaaggacgccacccccctggacttcgtgagagccaccgccaccatccccatccaggccagcctgcccttcggctggctgatcgtgggcgtggccctgctggccgtgttccagagcgccagcaagatcatcaccctgaagaagagatggcagctggccctgagcaagggcgtgcacttcgtgtgcaacctgctgctgctgttcgtgaccgtgtacagccacctgctgctggtggccgccggcctggaggccatgagcgacaacggcccccagaaccagagaaacgcccccagaatcaccttcggcggccccagcgacagcaccggcagcaaccagaacggcgagagaagcggcgccagaagcaagcagagaagaccccagggcctgcccaacaacaccgccagctggttcaccgccctgacccagcacggcaaggagggcctgaagttccccagaggccagggcgtgcccatcaacaccaacagcagccccgacgaccagatcggctactacagaagagccaccagaagaatcagaggcctgttcgccagaaccagaagcatgtggagcttcaaccccgagaccaacatcctgctgaacgtgcccctgcacggcaccatcctgaccagacccctgctggagagcgagctggtgatcggcgccgtgatcctgagaggccacctgagaatcgccggccaccacctgggcagatgcgacatcaaggacctgcccaaggagcccttcctgtacctgtacgccctggtgtacttcctgcagagcatcaacttcgtgagaatcatcatgagactgtggctgtgctggaagtgcagaagcaagaaccccctgctgtacgacgccaactacttcctgtgctggcacaccaactgctacgactactgcatcccctacaacagcgtgaccagcagcatcgtgatcaccagcggcgacggcaccaccagccccatcagcgagcacgactaccagatcggcggctacaccgcccccagcgccagcgccttcttcggcatgagcagaatcggcatggaggtgacccccagcggcacctggctgacctacaccggcgccatcaagctggacgacaaggaccccaacttcaaggaccaggtgatcctgctgaacaagcacatcgacgcctacaagaccttcccccccaccgagcccaagaaggacaagaagaagaaggcctacgagacccaggccctgccccagagacagaagaagcagcagaccgtgaccctgctgcccgccgccgacctggacgacttcagcaagcagctgcagcagagcatgagcagcgccgacagcacccaggccatcaccgtggccaccagcagaaccctgagctactacaagctgggcgccagccagagagtggccggcgacagcggcttcgccgcctacagcagatacagaatcggcaactacaagctgaacaccgaccacagcagcagcagcgacaacatcgccctgctggtgcagctgaacgagagcctgatcgacctgcaggagctgggcaagtacgagcagtacatcaagtggccctggtacatctggctgggcttcatcgccggcctgatcgccatcgtgatggtgtgataatag

SEQ ID NO: 60 - DNA, 5403 bp, Artificial Sequence
  Description of Artificial Sequence Synthetic polynucleotide
atgttcgtttttctggttctgctgccactcgtgtcaagtcagtgcgtgaaccttactacaaggactcagctcccaccagcatacacgaatagttttacgcggggcgtgtactatccagacaaagtgtttcgcagctctgttctacattcaactcaagacctgtttttgcctttcttctccaatgtgacctggttccacgccatccacgtgagtggcacgaacgggaccaaacggtttgataacccagtgctgccttttaacgacggggtatatttcgcctctactgaaaaatccaacatcatccgcggctggattttcgggaccactcttgactccaagacccagtcactcctgatcgtaaacaatgcgaccaacgtcgtgattaaggtgtgcgagtttcaattctgtaacgaccctttcctgggtgtatattaccacaagaataataagtcctggatggaatcagagtttagagtatactctagcgctaacaactgtacttttgaatatgtgtcccaacccttcttgatggacttggagggaaaacagggaaattttaagaatctccgagagttcgtgtttaaaaacattgacggctatttcaagatatactctaagcatacacccatcaatctggtccgcgatctgccacaggggtttagcgcactggaaccgttggtggatctccccattgggattaatatcacccgtttccagacacttttagccttgcatcggagctacctaacccccggggactcaagtagcggctggactgcgggagcggccgcctattatgtcggatatctgcagcctcggacattcctcctgaagtacaatgagaatggcacaattacagacgcagtagactgtgccctggatccgctctccgaaaccaaatgcacgctgaaatcatttacggtggaaaaaggtatataccagaccagcaatttcagggtgcagcctacggagtccattgtccgtttccccaatatcaccaatctgtgtcctttcggcgaagtgtttaacgcaactaggttcgcgagtgtctacgcctggaaccgaaagagaatctcaaactgtgtggccgattacagcgtcctgtacaactccgcatctttcagtaccttcaagtgctacggggtcagccccaccaaacttaacgatctttgcttcactaacgtttatgccgatagttttgtcatcaggggcgacgaagtgcgacagattgcccctggacagacgggaaagatcgccgactataactataagctgccagacgatttcacaggatgcgtgatcgcctggaatagcaacaatctggactctaaggtgggggggaattataattatttgtatagactgtttcgaaagtcaaaccttaagccatttgagagggatatcagcacagagatttaccaggcaggaagcaccccatgtaacggggtagaaggcttcaactgctacttccccctccagtcatatgggttccagcctaccaatggtgtgggttaccagccgtacagagtagtggttctttcatttgagctgctgcatgcccctgccaccgtctgcggacctaaaaaatctaccaatttagtgaaaaataagtgcgtgaattttaatttcaacggccttacgggcaccggcgtgctgactgagagcaataagaaattcttaccatttcagcagttcggccgcgacatagctgataccaccgatgcagttcgcgacccccagaccctggagatccttgacatcactccttgcagtttcggaggagtctcggtcatcacacctggaacaaacacatccaaccaggtggcagttctttaccaggatgtgaactgtaccgaagtgccagtggcaatccacgccgatcagttaactcccacctggagagtgtactctacaggctctaacgtcttccagactcgggccggttgccttattggggctgagcacgtgaacaactcctacgagtgcgacatacctattggtgccggcatctgtgccagctaccagacccaaaccaattcgccaaggcgagcgcgttctgtagcaagccagtcgattattgcctacactatgtccttaggtgctgagaactctgtggcttactctaacaactccatagcaattccaacaaactttacaattagtgttactactgaaatcttgcccgtcagcatgactaaaacctctgtcgattgtaccatgtacatttgtggggactctacagagtgcagcaatcttctgctccagtacggctctttttgtacgcagctgaaccgtgctctgactgggattgccgtcgagcaagataagaacacccaggaggtgtttgcccaagttaagcagatttataagacaccacccatcaaagacttcggcggatttaacttttctcagattctgcccgacccctccaagcccagcaagaggagctttatcgaggacctgctgttcaataaggtcacactcgctgatgcaggattcatcaagcagtatggcgattgcctcggagacatcgctgcgagagacctcatatgcgctcagaaattcaatggcctgacggtgctacctccgctactgactgacgaaatgatagctcagtacacgtcggctctcttggccggaacaatcacctccggatggacctttggcgcgggagcagcactacagatcccttttgcaatgcaaatggcttaccggttcaatggcataggggtaactcaaaatgtgctgtacgagaaccaaaaattgatcgctaaccagttcaacagcgccattgggaagatccaggattctttgtcctcaaccgccagtgcattgggcaagctccaggacgtcgttaaccagaacgctcaggccttaaacacgctggtcaaacagttgtcctccaattttggcgctatatccagcgttctcaatgatatcctttcccgcttagatccaccagaagctgaggtccaaattgataggttaataaccggcagactccagagcctgcaaacttacgtcacccagcaactcatacgcgccgcggagatccgcgctagcgcaaacctagctgccactaaaatgagtgagtgtgttctcggacagtctaagcgggtggacttttgcggcaaaggctatcacctcatgagcttcccccaaagcgcaccacatggcgttgtgttcttgcacgtgacttacgttcccgcacaggagaaaaatttcaccacagcccccgccatctgccatgatgggaaagctcattttccacgagaaggggtgttcgtgtcaaacggtacacactggtttgtcacacaaagaaatttttatgaacctcaaattatcacaactgacaataccttcgtgagcggaaactgtgatgtcgtaattgggatcgtaaacaacactgtgtatgacccccttcagcccgaactggacagtttcaaggaagagcttgacaagtatttcaagaaccatacttcaccagacgtagaccttggtgatatttcaggaatcaacgctagtgtggtgaacatccagaaagaaattgatcgcctcaatgaggtcgcgaaaaatctgaatgagtctctgatcgacctgcaagagttggggaagtacgaacagtatattaaatggccctggtacatttggctgggatttatagctggactcattgccattgttatggtcacaataatgctgtgttgcatgactagctgttgctcatgcctaaaagggtgctgcagttgcggctcctgttgcaagtttgatgaagacgatagcgagccggtccttaaaggcgttaagctacattatactagggctaagagatccggcagtggggcgaccaactttagcttgttgaagcaagcaggggacgtggaagaaaaccccggcccttacgccctagtgtactttctgcagtccattaacttcgttcggattatcatgaggttgtggctgtgttggaagtgtcggtcgaagaatccactcctgtacgatgcaaattactttctgtgctggcacggagacggcaaaatgaaagacctgtccccgagatggtatttttattatctgggtaccggtcccgaggcggggctgccctacggggcaaacaaagacggaatcatctgggtcgcaacagagggagctcttaatacacctaaagatcacattggcacccgcaatcccgagaagtactgtgccctggcccccaatatgatggtgacaaataacacctttacattaaagggcggggccccaaccaaggtgacattcggtacatacaagaatacctgtgacggcacaacgttcacgtatgcaagcgctctgtgggagatccaacaggtggtggacgccgacctgaatgaaagtctgattgatctccaggaactcggcaaatatgagcagtatatcaagtggccttggtacatctggctcggttttatcgctggtcttatcgccatcgtgatggtgcagactgcttgcaccgatgataatgcactcgcgtactacaacaccacaaaaggaggacgatttgtgctagccctgctcagtgatctgcaagatctcaaatgggcccgcttcccgaagtccgatggaaccggcacaatctatacagaattggaacctccttgtaggttcgtgaccgatactcccaagggtcccaaggtaaaatacctgcgggtagaagcttttgaatactaccacactactgacccatcttttctgggcagatatatgtctgcattaaatcacaccaaaaagtggataacagtggccacctcccggacactgtcatactataaactgggtgcatcccagcgggttgctggtgattccggattcgccgcctattcgcggtatagaatagggaattacaagttgaataccgaccactccagttctagtgataacatagccctgctggttcaggttcttcagcagctgagagtagaatcttccagcaagctgtgggcccagtgtgttcaactccacaatgatattttactcgccaaggacactgcaccgtcagcctctgccttcttcgggatgtctcgtattggtatggaggttactcctagcggcacatggctgacgtacaccggggctataaagttggacgacaaggacccaaacttcaaggaccaagtgatcttactgaacaaacatatcgatgcttataagacattccctcctactgagcctaaaaaagataaatcaaagctcattgagtacacagattttgctacaagcgcttgtgtcctggcggccgagtgcaccatcttcaaagacgctagtggcaagcccgtgccgtattgctatgacaccaatgtgctcgagggttcagtcgcctatgagtcattaaggccagatacgaggtacgtcctaatggatgggtag

SEQ ID NO: 61 - protein, 20 aa, Artificial Sequence
  Description of Artificial Sequence Synthetic peptide
  SITE (1)..(10): This region may encompass 0-10 residues
  SITE (11)..(20): This region may encompass 0-10 residues
  See specification as filed for detailed description of substitutions and preferred embodiments
GGGGGGGGGGSSSSSSSSSS

SEQ ID NO: 62 - protein, 10 aa, Artificial Sequence
  Description of Artificial Sequence Synthetic peptide
GGGGSGGGGS

SEQ ID NO: 63 - protein, 8 aa, Artificial Sequence
  Description of Artificial Sequence Synthetic peptide
GGGSGGGS

SEQ ID NO: 64 - protein, 11 aa, Artificial Sequence
  Description of Artificial Sequence Synthetic peptide
GGGSGGGSGGS

SEQ ID NO: 65 - protein, 4 aa, Artificial Sequence
  Description of Artificial Sequence Synthetic peptide
SGSG